(19) United States
(12) Patent Application Publication
(10) Pub. No.: US 2012/0264810 A1
(43) Pub. Date: Oct. 18, 2012

(54) COMPOSITIONS AND METHODS FOR ENHANCING CELLULAR UPTAKE AND INTRACELLULAR DELIVERY OF LIPID PARTICLES

(75) Inventors: Paulo J.C. Lin, Vancouver (CA); Yuen Yi c. Tam, Vancouver (CA); Srinivasulu Masuna, Edmonton (CA); Marco A. Ciufolini, Vancouver (CA); Michel Roberge, Vancouver (CA); Pieter R. Cullis, Vancouver (CA)

(73) Assignee: The University of British Columbia, Vancouver (CA)

(21) Appl. No.: 13/497,395

(22) PCT Filed: Sep. 22, 2010
(86) PCT No.: PCT/IB2010/002518

§ 371 Date: Jul. 3, 2012

Related U.S. Application Data
(60) Provisional application No. 61/277,306, filed on Sep. 22, 2009.
(60) Provisional application No. 61/277,307, filed on Sep. 22, 2009.
(60) Provisional application No. 61/400,763, filed on Jul. 30, 2010.
(60) Provisional application No. 61/400,758, filed on Jul. 30, 2010.

(51) Int. Cl. C07J 41/00; A61K 47/24; C12N 5/071; A61K 31/713; A61K 47/22; C07D 215/46; A61K 31/7105

(52) U.S. Cl. 514/44 A; 540/5; 546/163; 514/788; 514/44 R; 435/375

(57) ABSTRACT

Compositions, methods and compounds useful for enhancing the uptake of a lipid particle b\ a cell are described In particular embodiments, the methods of the invention include contacting a cell with a lipid particle and a compound that binds a Na+/K+ ATPase to enhance uptake of the lipid particle b\ the cell Related compositions useful in practicing methods include lipid particles comprising a conjugated compound that enhances uptake of the lipid particles b\ the cell The methods and compositions are useful in delivering a therapeutic agent to a cell, e g for the treatment of a disease or disorder in a subject

### CLAIM OF PRIORITY

This application claims priority to provisional U.S. Application Nos. 61/277,306, filed 22 Sep. 2009; 61/277,307, filed 22 Sep. 2009; 61/400,758, filed 30 Jul. 2010; and 61/400,763, filed 30 Jul. 2010; each of which is incorporated by reference in its entirety.

### STATEMENT OF GOVERNMENT INTEREST

This invention was made with government support under University-Industry grant 59836 awarded by the Canadian Institutes for Health Research (CIHR). The government may have certain rights in this invention.

### TECHNICAL FIELD

The present invention is related to the delivery of lipid particles, including those comprising a therapeutic agent, to cells.

### BACKGROUND

The use of siRNA for in vivo applications requires sophisticated delivery technologies, as “naked” siRNA molecules are rapidly broken down in biological fluids, are rapidly cleared from the circulation, do not accumulate at disease sites and cannot penetrate target cell membranes to reach their intracellular sites of action (reviewed in Zhang et al., 2007). Liposomal nanoparticle (LN) formulations of siRNA have demonstrated significant potential for overcoming these problems and enabling siRNA molecules to be used as therapeutics (Zimmerman et al. 2006). However, the design of LN formulations of siRNA for in vivo applications is far from optimized.

LN systems are accumulated into cells by endocytosis (Basha et al., in preparation; Lin et al., in preparation) and encapsulated material such as siRNA must then be released from the endosomes to be active. Methods of enhancing uptake into specific cells and then delivering siRNA into the cytosol remain a challenge. Targeting protocols involving macromolecules such as monoclonal antibodies (MAb), MAb fragments or peptides result in targeted LN systems that are expensive, difficult to manufacture, irreproducible, are often rapidly cleared on i.v. injection and are usually immunogenic. Other approaches such as fusogenic “virosomes” made with ultra violet-inactivated Sendai virus (Kunisawa et al., 2005) or using ultrasound to burst internalized LNs (Kinoshita and Hynynen, 2005, Negishi et al., 2008) suffer from similar difficulties.

Accordingly, there remains a need in the art for new compounds and methods for enhancing LN uptake and cytosolic delivery into target cells.

Silencing of specific disease-associated genes mediated by small interference RNA (siRNA) in vitro has shown promise for disease treatment ((Dorsett and Tuschl, 2004); (de Fougerolles et al., 2007)). However, the therapeutic potential of this treatment has been limited by obstacles in delivering siRNA to target diseased cells. The use of siRNA for in vivo applications requires sophisticated delivery technologies, as “naked” siRNA molecules are rapidly broken down in biological fluids and cannot penetrate cell membranes. Liposomal nanoparticle (LN) encapsulation of siRNA has demonstrated significant potential for overcoming these problems for delivery of siRNA to hepatocytes in vivo and thus enabling siRNA to be used as therapeutics ((Zimmermann et al., 2006)). However, the design of LN formulations of siRNA (LN-siRNA) for other in vivo applications is far from optimized. In particular, effective targeting to specific cells is lacking as the majority of systemic administered LN-siRNA is taken up by the reticular endothelial system in the spleen and liver (Fenske et al., 2008).

Targeting ligands such as antibody fragments and peptides against specific cell surface receptors have been used to deliver liposomes to specific cells ((Sapra and Allen, 2003)). However, induction of immune responses to the targeting ligand, cost and formulation issues encountered with proteins or peptides indicates an urgent need for better targeting ligands. Small molecule targeting ligands conjugated to lipid anchors in LN offer important potential advantages, notably much reduced immunogenicity and ease of LN manufacture. This potential has been demonstrated for anisamide which possesses high affinity for sigma receptors and has been shown to increase delivery of LN to prostate and lung cancer cells which overexpress sigma receptors ((Banerjee et al., 2004); (Li and Huang, 2006)).

Clearly there remains a need for new molecules capable of enhancing the cellular uptake of agents, including therapeutic agents, by cells.

### SUMMARY

In one embodiment, the present invention provides a method of enhancing cellular uptake of a lipid particle, comprising contacting a cell with a lipid particle and a compound that binds a Na+/K+-ATPase. In particular embodiments said contacting occurs in vitro or in vivo. In certain embodiments, the cell is a mammalian cell, e.g., a human cell. In certain embodiments, said lipid particle comprises a therapeutic agent.

In a related embodiment, the present invention provides a method of treating or preventing a disease or disorder in a subject, comprising providing to the subject a compound that binds a Na+/K+-ATPase and a lipid particle comprising a therapeutic agent. In particular embodiments, the subject is a mammal, e.g., a human.

In a further related embodiment, the present invention includes a lipid particle comprising a compound that binds a Na+/K+-ATPase, wherein said compound is conjugated to the lipid particle. In one embodiment, said compound is conjugated to a lipid component of said lipid particle.

In various embodiments of methods and compositions of the present invention, said NA+/K+-ATPase is a cardiac glycoside. In particular embodiments, said cardiac glycoside is selected from the group consisting of helveticoside, digydroouabain, digitoxigenin, strophanthidin, lanatoside C, ditoxigenin, digoxin, ouabain, and proscillaridin A.

In various embodiments of methods and compositions of the present invention, said lipid particle comprises: a cationic lipid; one or more non-cationic lipids; and a conjugated lipid that inhibits aggregation of particles. In certain embodiments, said lipid particle further comprises cholesterol. In certain embodiments, the cationic lipid is selected from DLin-K-DMA, DLinDMA, and DLinDAP. In certain embodiments, the one or more non-cationic lipids are selected from the group consisting of: DOPE, POPC, EPC, DSPC, cholesterol, and a mixture thereof. In certain embodiments, the conjugated lipid is a PEG-lipid.

In particular embodiments of methods and compositions of the present invention, the therapeutic agent is an interfering RNA. In one embodiment, the interfering RNA is a siRNA.

In certain embodiments, the compound that binds a Na+/K+-ATPase is conjugated to the lipid particle.

In a related embodiment, the present invention provides a conjugate comprising a compound that binds a Na+/K+-ATPase and a lipid. In certain embodiments, the compound that binds a Na+/K+-ATPase is a cardiac glycoside. In particular embodiments, the cardiac glycoside is selected from the group consisting of: helveticoside, dihydroouabain, digoxigenin, strophanthidin, lanatoside C, ditoxigenin, digoxin, ouabain, and proscillaridin A. In one embodiment, the cardiac glycoside is ouabain. In another embodiment, the cardiac glycoside is strophanthidin. In a particular embodiment, said conjugate is the lipid conjugate Example 9. In particular embodiments of the conjugate, the lipid is a phospholipid. In certain embodiments, the phospholipid is a PEG-functionalized phospholipid. In certain embodiments, the phospholipid comprises a PEG moiety. In particular embodiments, said compound that binds Na+/K+-ATPase induces endocytosis.

A conjugated lipid can have the formula:

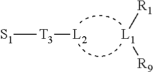

wherein
S₁ includes a quinoline moiety or a moiety that binds to Na+/K+-ATPase;
R¹ is a C₁₀ to C₃₀ group having the formula

-L^{1a}-(CR^{1a}R^{1b})_{α}-[L^{1b}-(CR^{1a}R^{1b})_{β}]_{γ}-L^{1c}-R^{1c},

wherein
L^{1a} is a bond, —CR^{1a}R^{1b}—, —O—, —CO—, —NR^{1d}—, —S—, or a combination thereof; each R^{1a} and each R^{1b}, independently, is H; halo; hydroxy; cyano; C₁-C₆ alkyl optionally substituted by halo, hydroxy, or alkoxy; C₃-C₈ cycloalkyl optionally substituted by halo, hydroxy, or alkoxy; —OR^{1c}; —NR^{1c}R^{1d}; aryl; heteroaryl; or heterocyclyl;
each L^{1b}, independently, is a bond, —(CR^{1a}R^{1b})₁₋₂—, —O—, —CO—, —NR^{1d}—, —S—,

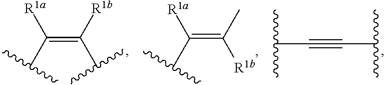

or a combination thereof; or has the formula

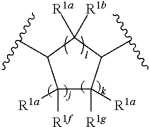

wherein j, k, and 1 are each independently 0, 1, 2, or 3, provided that the sum of j, k and 1 is at least 1 and no greater than 8; and R^{1f} and R^{1g} are each independently R^{1b}, or adjacent R^{1f} and R^{1g}, taken together, are optionally a bond;
or has the formula

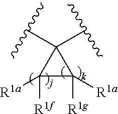

wherein j and k are each independently 0, 1, 2, 3, or 4 provided that the sum of j and k is at least 1; and R^{1f} and R^{1g} are each independently R^{1b}, or adjacent R^{1f} and R^{1g}, taken together, are optionally a bond;
or has the formula:

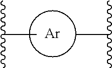

wherein —Ar— is a 6 to 14 membered arylene group optionally substituted by zero to six R^{1a} groups;
or has the formula:

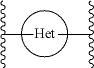

wherein -Het- is a 3 to 14 membered heterocyclylene or heteroarylene group optionally substituted by zero to six R^{1a} groups;
L^{1c} is —(CR^{1a}R^{1b})₁₋₂—, —O—, —CO—, —NR^{1d}—, —S—,

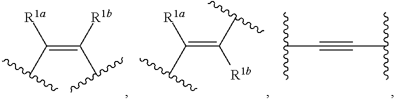

or a combination thereof;
R^{1c} is H; halo; hydroxy; cyano; C₁-C₆ alkyl optionally substituted by halo, hydroxy, or alkoxy; C₃-C₈ cycloalkyl optionally substituted by halo, hydroxy, or alkoxy; aryl; heteroaryl; or heterocyclyl; or R^{1c} has the formula:

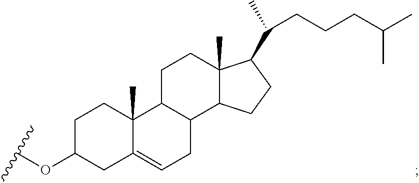

R^{1d} is H; halo; hydroxy; cyano; C₁-C₆ alkyl optionally substituted by halo, hydroxy, or alkoxy; C₃-C₈ cycloalkyl optionally substituted by halo, hydroxy, or alkoxy; aryl; heteroaryl; or heterocyclyl;
α is 0-6;
each β, independently, is 0-6; and
γ is 0-6;

### R⁹ is R¹ or R⁵;

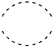

represents a connection between L₂ and L₁ which is:
(1) a single bond between one atom of L₂ and one atom of L₁, wherein
   L₁ is C(Rₐ), O, S or N(Q);
   L₂ is —(CR₅R₆)ₓ—, —C(O)—(CR₅R₆)ₓ—, —(CR₅R₆)ₓ—CR₅═CR₅—(CR₅R₆)_{y}—, —C(O)—(CR₅R₆)ₓ—CR₅═CR₅—(CR₅R₆)_{y}—, —O—, —S—, —N(Q)-, ═N—, ═C(R₅)—, —CR₅R₆—O—, —CR₅R₆—N(Q)-, —CR₅R₆—S—, —C(O)N(Q)-, —C(O)O—, —N(Q)C(O)—, —OC(O)—, —C(O)—, or —X—C(R₅)(YR₃)—;
      wherein X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
   Rₐ is H, alkyl, alkoxy, —OH, —N(Q)Q, or —SQ;
(2) a double bond between one atom of L₂ and one atom of L₁, wherein
   L₁ is C;
   L₂ is —(CR₅R₆)ₓ—CR₅═, —C(O)—(CR₅R₆)ₓ—CR₅═, —N(Q)═, —N—, —O—N═, —N(Q)-N═, or —C(O)N(Q)-N═;
(3) a single bond between a first atom of L₂ and a first atom of L₁, and a single bond between a second atom of L₂ and the first atom of L₁, wherein
   L₁ is C or C(Rₐ)—(CR₅R₆)ₓ—C(Rₐ);
   L₂ has the formula

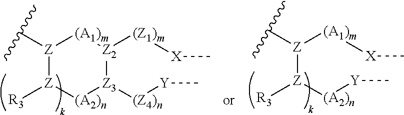

wherein
   X is the first atom of L₂, Y is the second atom of L₂, - - - - - represents a single bond to the first atom of L₁, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
   Z₁ and Z₄ are each, independently, —O—, —S—, —CH₂—, —CHR⁵—, or —CR⁵R⁵—;
   Z₂ is CH or N;
   Z₃ is CH or N;
   or Z₂ and Z₃, taken together, are a single C atom;
   A₁ and A₂ are each, independently, —O—, —S—, —CH₂—, —CHR⁵—,
   or —CR⁵R⁵—;
   each Z is N, C(R₅), or C(R₃);
   k is 0, 1, or 2;
   each m, independently, is 0 to 5;
   each n, independently, is 0 to 5;
   where m and n taken together result in a 3, 4, 5, 6, 7 or 8 member ring;
(4) a single bond between a first atom of L₂ and a first atom of L₁, and a single bond between the first atom of L₂ and a second atom of L₁, wherein
   (A) L₁ has the formula:

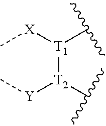

wherein
   X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
   T₁ is CH or N;
   T₂ is CH or N;
   or T₁ and T₂ taken together are C═C;
   L₂ is CR₅; or
(B) L₁ has the formula:

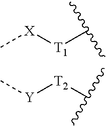

wherein
   X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
   T₁ is —CR₅R₆—, —N(Q)-, —O—, or —S—;
   T₂ is —CR₅R₆—, —N(Q)-, —O—, or —S—;
   L₂ is CR₅ or N;
   each of x and y, independently, is 0, 1, 2, 3, 4, or 5;
      T₃ is a bond or -L₆-(CR₅R₆)ₘ-L₇-[(CR₅R₆)ₚO]_{q}-L₈-(CR₅R₆)ₙ-L₉- wherein

L₆ is a bond, —CR^{1a}R^{1b}—, —O—, —CO—, —NR^{1d}—, —S—, —P(=Q₁)(Q₂)-, or a combination thereof;

L₇ is a bond, —CR^{1a}R^{1b}—, —O—, —CO—, —NR^{1d}—, —S—, —P(=Q₁)(Q₂)-, or a combination thereof;

L₈ is a bond, —CR^{1a}R^{1b}—, —O—, —CO—, —NR^{1d}—, —S—, —P(=Q₁)(Q₂)-, or a combination thereof;

L₉ is a bond, —CR^{1a}R^{1b}—, —O—, —CO—, —NR^{1d}—, —S—, —P(=Q₁)(Q₂)-, or a combination thereof;

m is 0 to 10;

n is 0 to 10;

p is 1 to 6;

q is 0 to 2000;

each occurrence of R₅ and R₆ is, independently, H, halo, cyano, hydroxy, amino, alkyl, alkoxy, cycloalkyl, aryl, heteroaryl, or heterocyclyl;
each Q, independently, is H, alkyl, acyl, cycloalkyl, alkenyl, alkynyl, aryl, heteroaryl or heterocyclyl;
each Q₁, independently, is O or S; and
each Q₂, independently, is —OQ, —SQ, —N(Q)Q, alkyl, or alkoxy;
or a pharmaceutically acceptable salt thereof.

The quinoline moiety can include a 4-aminoquinoline, an 8-aminoquinoline, or a 4-methanolquinoline. The quinoline moiety can include a chloroquine moiety, an amodiaquine moiety, a primaquine moiety, a pamaquine moiety, a mefloquine moiety, a quinine moiety, or a quinidine moiety.

The moiety that binds to Na+/K+-ATPase can include a cardiac glycoside moiety. The cardiac glycoside moiety includes a helveticoside moiety, a dihydroouabain moiety, a digitoxigenin moiety, a strophanthidin moiety, a lanatoside C moiety, a ditoxigenin moiety, a digoxin moiety, a ouabain moiety, a proscillaridin A moiety, an arenobufagin moeity, a bufotalin moiety, a cinobufagin moiety, a marinobufagin moiety, a scilliroside moiety, an acetyldigitoxin moiety, an acetyldigoxin moiety, a lanatoside C moiety, a deslanoside moiety, a medigoxin moiety, a gitoformate moiety, a daigremontianin moiety, a cymarin moiety, or a peruvoside moiety.

S₁ can have the formula: -G-S₃-Lc, wherein G is a bond, —O— or a glycosidic linkage, S₃ is a steroid structure, and Lc is a lactone. S₁ can have the structure:

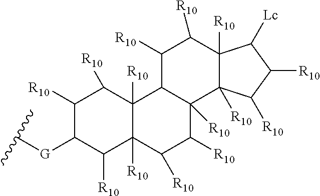

where each R₁₀, independently, is H, OH, CH₃, CHO, C(O)CH₃, oxo, or two adjacent R₁₀, taken together, are a double bond or an epoxide. G can be a bond, —O—, or can have the formula

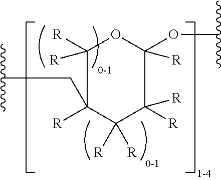

where each R, independently, is H, OH, alkyl, alkoxy, acyl, NH₂, or NH-acyl.

Lc can have the formula:

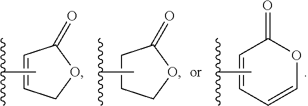

S₁ can have the formula:

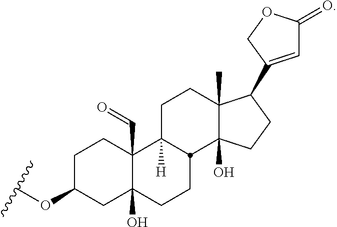

The lipid can have the formula:

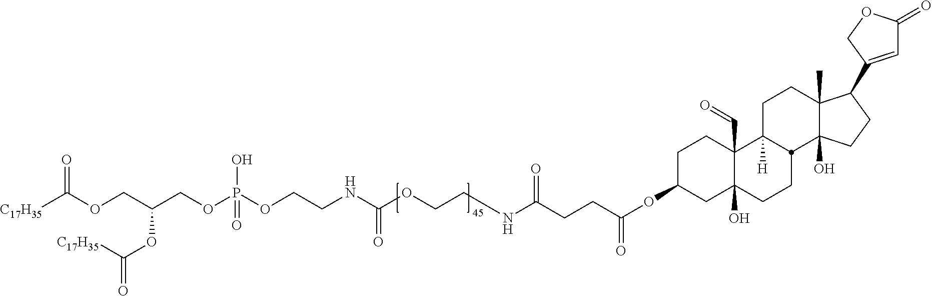

S₁ can have the formula:

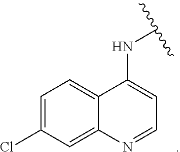

The lipid can have the formula:

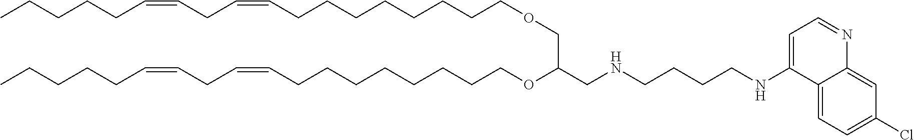

In another aspect, a lipid particle can include a lipid as described above. The lipid particle can further include a cationic lipid, a neutral lipid, and a lipid capable of reducing aggregation.

The neutral lipid can be selected from DSPC, DPPC, POPC, DOPE, or SM; the lipid capable of reducing aggregation is a PEG lipid. The lipid particle can further include a sterol.

The lipid particle can include an active agent. The active agent can be a nucleic acid selected from the group consisting of a plasmid, an immunostimulatory oligonucleotide, an siRNA, an antisense oligonucleotide, a microRNA, an antagomir, an aptamer, and a ribozyme.

In another aspect, a pharmaceutical composition can include the lipid particle and a pharmaceutically acceptable carrier.

In another aspect, a method for enhancing cellular uptake of a nucleic acid includes contacting a cell with: a compound selected from the group consisting of: levodopa, naphazoline hydrochloride, acetohexamide, niclosamide, diprophylline, and isoxicam; and a lipid particle comprising a nucleic acid.

In another aspect, a method for enhancing cytosolic distribution of a nucleic acid, comprising contacting a cell with: a compound selected from the group consisting of: azaguanine-8, isoflupredone acetate, chloroquine, trimethobenzamide, hydrochloride, isoxsuprine hydrochloride, and diphemanil methylsulfate; and a lipid particle comprising a nucleic acid.

In another aspect, a method of enhancing cellular uptake of a lipid particle, comprising contacting a cell with a lipid particle and a compound that binds a Na+/K+-ATPase. The compound that binds a Na+/K+-ATPase can be a lipid as described above.

### BRIEF DESCRIPTION OF THE DRAWINGS

FIG. 1. (A) Uptake of siRNACy3 encapsulated with DLinDMA. 10,000 Raw 264.7 cells were treated with varying concentration of siRNACy3 at 1, 2, 8 and 24 hours. Cy3 signal was quantitated with the Cellomics ArrayScan VTI. Bars are represented as ±standard deviation of triplicates. (B and C) 10 μg/mL of siRNACy3 encapsulated with DLinDMA were co-treated with 81 different drugs for 24 hours. In (B) total intensity of Cy3 in the presence of drug was normalized to total intensity of Cy3 in the absence of drug. Normalized values were ranked. In (C) the percentage of punctate intensity of Cy3 in the presence of drug was normalized to the percentage of punctate intensity of Cy3 in the absence of drug. Normalized values were ranked.

FIG. 2. Raw 264.7 cells were treated with 10 μg/mL of siRNACy3 encapsulated in LNs in the presence of 0, 0.1, 0.3, 1, 3, 10 and 30 μM of (A) diprophylline or isoxicam, or (B) chloroquine, trimethobenzamide hydrochloride or diphemanil methylsulfate. (C) Raw264.7 cells were treated with DLinDMA LNs for 24 hours in the presence or absence of chloroquine. Images were acquired using Cellomics Arrayscan VTI.

FIG. 3. Structure and synthesis of a lipid incorporating a chloroquine motif.

FIG. 4. (A) LN uptake was assessed by SPDiO fluorescence using Cellomics. Cells were incubated for 16 hours. (B) Distribution of Cy5-siRNA was assessed by Cellomics. Raw 264.7 cells were treated for 16 hours with DLinDMA LNs in the presence or absence of free chloroquine or DLinDMA LNs formulated with 5% mole CQ-lipid. (C) Same LN formulations as in (B) were incubated for 24 hours and siRNA-Cy5 fluorescence was examined. (D) Uptake of LNs by clathrin mediated endocytosis was monitored by using Transferrin-594 internalization (red). Cells were imaged for SPDiO at 488 nm (green). Images collected from red and green channels were merged and displayed. Longer image acquisition time was used for cells incubated with LNs containing CQ-lipid. (E) The same images were acquired at 633 nm to visualize the distribution of Cy5-siRNA (red). Longer image acquisition time was again used for cells incubated with LNs containing CQ-lipid.

FIG. 5: Raw264.7 cells were treated for 24 hours with siRNACy3 encapsulated in DLinDMA LNs in the presence or absence of 5% CQ-lipid. A lipid label, SPDiO, was incorporated into the LN to monitor uptake. LN uptake was assessed by (A) SPDiO fluorescence and distribution of siRNA was assessed by (B) Cy3 fluorescence using the Cellomics ArrayScan VTI. (C) Raw264.7 cells were incubated with the same LN formulations as in (B) for 24 hours at 10 μg/mL siRNACy3. siRNACy3 distribution was examined by confocal microscopy. (D) GAPDH siRNA was formulated into LN particles comprising of 40% DLinDMA with 0% CQ-lipid or 40% DLinDMA with 5% CQ-lipid. Raw264.7 cells were treated with 5, 10 and 20 μg/mL of siRNA for 48 and 72 hours. GAPDH protein expression was assessed by western blotting against anti-GAPDH and anti-actin. (E) LNCaP cells were treated accordingly as in (D).

FIG. 6 provides a table showing siRNACy3 accumulation and cytosolic distribution associated with various compounds.

FIG. 7A shows the quantification of liposome uptake in 96-well format. Cells were grown in 96-well optical plate for 24 hr. Chemical compounds and LNP were added and incubated at 37° C. Automated fluorescence microscopy was performed using a Cellomics Arrayscan. Representative images of MDCK cells are shown. Individual object segmentation based on the nuclear stain (Hoechst's stain), mask encompassing the cytoplasm and quantification of SPDiO and siRNA-Cy3 uptake were performed using the Cellomics Compartmental Analysis algorithms. FIG. 7B shows the progressive uptake of liposomes over time. HeLa cells were grown in 96-well optical plates for 24 hr before liposome treatment (5 μg/mL of siRNA-Cy3) for 3, 8 and 24 hr. SP-DiO and siRNA-Cy3 uptake were quantified using the Cellomics Compartmental Analysis algorithms. All values are means±SD of 4 experiments.

FIG. 8A shows normalized SP-DiOC18 fluorescence values for 800 small molecules. HeLa cells were incubated with 800 small molecules and LN-siRNA for 24 h. Cellular SPDiO-C₁₈ fluorescence caused by individual compound was normalized to SP-DiOC₁₈ fluorescence in cells untreated with any compound. Fluorescence values were sorted from the highest to the lowest. FIG. 8B shows effects of cardiac glycosides on LNP uptake. HeLa cells were incubated with 50 μg/ml (lipid concentration) of empty LNP and each of 9 cardiac glycosides at 0.05 μM, 0.15 μM and 1.5 μM for 24 h. Cardiac glycosides on the x-axis are arranged by their affinity to the Na+/K+ ATPase, from the weakest (helveticoside) to the strongest (Proscillaridin A) (Paula et al., 2005). Cellular SPDiO fluorescence caused by individual compound was normalized to SPDiO fluorescence in cells untreated with any cardiac glycoside. All values are means±SD of 4 experiments.

FIG. 9A shows uuabain induces LNP uptake in HeLa cells. Confocal micrographs of HeLa cells treated with 10 μg/ml of siGAPDH-LNP and 0 nM or 30 nM of ouabain for 24 h. Cell nuclei were stained with Hoechst's dye in blue. SPDiO fluorescence is shown in green. FIG. 9B shows GAPDH expression is reduced in the presence of 30 nM ouabain. Cells were treated with or without 10 ug/ml of siGAPDH-LNP or siScramble-LNP in the presence of 0 nM or 30 nM of ouabain for 24 h. LNP and ouabain were removed and cells were further incubated in fresh medium for 48 hrs. Equal portions of protein samples were analyzed by immunoblotting to GAPDH and β-actin which served as a loading control.

FIG. 10 shows synthesis of STR-PEG. A handle for the conjugation of strophanthidin (1) to a readily available PEG-functionalized phospholipid (DSPE-PEG-NH2) was installed treating cardenolide 1 with succinic anhydride in the presence of 4-dimethylaminopyridine (DMAP) at room temperature to furnish carboxylic acid 2 in high yield. Where conventional peptide coupling methods failed, exposure of succinate 2 to Yamaguchi's reagent in pyridine furnished the mixed anhydride, which directly treated with DSPE-PEG-NH2 and DMAP, giving lipid conjugate 3 (STR-PEG) after careful chromatography on silica gel.

FIG. 11 demonstrates that more targeted LNP are taken up by cells. FIG. 11A provides representative images of HeLa cells treated with targeted LNP containing strophanthidin-PEG (STR-PEG) or control LNP. Cell nuclei were stained with Hoechst's dye in blue. SPDiO fluorescence is shown in green. FIG. 11B provides a graph showing spDio fluorescence associated with various concentrations of LNP comprising STR-PEG or control DSPE-PEG in HeLa cells. FIG. 11C provides a graph showing spDio fluorescence associated with various concentrations of LNP comprising STR-PEG or control DSPE-PEG in LNCaP cells.

FIG. 12A shows LNP uptake is dependent on ATP1A1. Wild-type HeLa cells or cells stably transfected with shATP1A1 or shScramble plasmid were treated with STR-PEG-LNP (DLinK-C2-DMA/DSPC/Cholesterol/PEG-s-DMG/STR-PEG/SPDiO at 40/14.8/40/4/1/0.2 mol %) or DSPE-PEG-LNP (DLinK-C2-DMA/DSPC/Cholesterol/PEG-s-DMG/DSPE-PEG/SPDiO at 40/14.8/40/4/1/0.2 mol %) at 25 μg/ml of lipid concentration for 24 hrs. Confocal images were collected and SPDiO fluorescence was quantified using ImageJ. All values are means±SD of 3 experiments. FIG. 12B shows ATP1A1 expression is knocked down in stable HeLa cell lines. Cells stably transfected with or without shATP1A1 or shScramble plasmid were lysed. Equal portions of protein samples were analyzed by immunoblotting to ATP1A1 and β-actin.

FIG. 13A shows GAPDH expression is reduced in the presence of STR-PEG-LNP. (A) Cells were treated with STR-PEG-LNP (DLinK-C2-DMA/DSPC/Cholesterol/PEG-s-DMG/STR-PEG/SPDiO at 40/17.3/40/1.5/1/0.2 mol %) or DSPE-PEG-LNP (DLinK-C2-DMA/DSPC/Cholesterol/PEG-s-DMG/DSPE-PEG/SPDiO at 40/17.3/40/1.5/1/0.2 mol %) encapsulation siGAPDH at various siRNA concentration indicated for 72 hrs. Equal portions of protein samples were analyzed by immunoblotting to GAPDH and β-actin which served as a loading control. FIG. 13B shows GAPDH and β-actin intensities in western blots quantification using ImageJ. GAPDH levels were normalized to that of β-actin and reported relative to the untreated control group.

FIG. 14 shows silencing of GAPDH in mouse liver and kidney. Three mice per group were treated with PBS, STR-PEG-LNP (DLinK-C2-DMA/DSPC/Cholesterol/PEG-s-DMG/STR-PEG at 40/10/40/5/5 mol %) and DSPE-PEG-LNP (DLinK-C2-DMA/DSPC/Cholesterol/PEG-s-DMG/DSPE-PEG at 40/10/40/5/5 mol %) encapsulating siGAPDH. mRNA levels of GAPDH in the liver and kidney were quantified by qRT-PCR in triplicates and normalized to the PBS control group. Error is expressed as the standard deviation of the mean relative quantity of the animals in each treatment group.

### DETAILED DESCRIPTION

Small molecules that enhance uptake and intracellular delivery of liposomal nanoparticles (LN) containing nucleic acids, e.g., siRNA, into target cells have been identified, and lipid molecules derived from these small molecules that enhance the delivery properties of LN systems have been developed. As described in the accompanying Examples, two classes of small molecules were identified: (1) molecules that enhanced uptake of LN nucleic acids; and (2) molecules that enhanced the cytosolic distribution of LN nucleic acids. In addition, a novel lipid incorporating a chloroquine motif in the headgroup was synthesized (CQ-lipid), and it was shown that this CQ-lipid enhanced cytosolic delivery of encapsulated nucleic acids when it was included in LN formulations.

Ligand-mediated targeting of liposomal drugs to diseased cells could be an effective strategy for increasing therapeutic benefits. However, immunogenicity and formulation issues encountered with ligands such as antibodies, antibody fragments or peptides indicates a need for improved targeting ligands. The present invention is based, in part, upon the identification of small chemical molecules that enhance cellular uptake of liposomal nanoparticles (LNs). As described in the accompanying Examples, high throughput screening of 800 small molecules in 6 mammalian cell lines was performed to identify small molecules that enhanced cellular uptake of LNs. Molecules that caused the highest uptake of LNs in human cervical cancer (HeLa) cells included members of the cardiac glycoside family. For example, confocal microscopy confirmed the presence of substantial amount of LNs in HeLa cells treated with helveticoside, a cardiac glycoside, for 24 hrs. Accordingly, compositions and methods for enhancing the cellular uptake of LNs are provided.

Compounds that Enhance Uptake and Cytosolic Distribution of Lipid Particles

Compounds that enhance uptake and cytosolic distribution of lipid particles include:

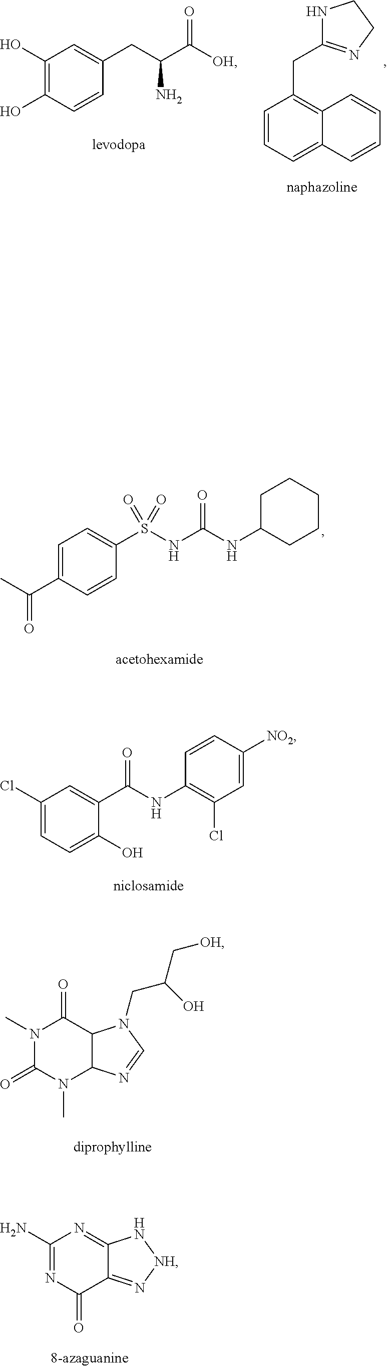

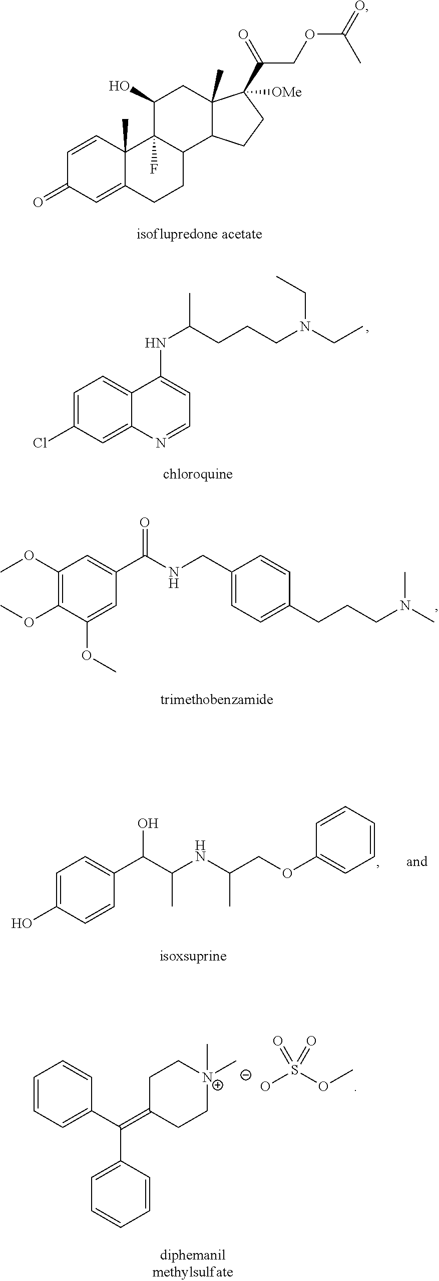

Compounds that Bind a Na+/K+-ATPase

Methods and compositions may utilize any compound that binds a Na+/K+-ATPase. In one embodiment, the compound induces or enhances endocytosis of the Na+/K+-ATPase.

In particular embodiments, the compound that binds a Na+/K+-ATPase is a cardiac glycoside. Cardiac glycosides are a diverse family of naturally derived molecules. Members of this family have been used in treatment of heart failure for many years ((Schoner and Scheiner-Bobis, 2007)). (Although referred to as “glycosides”, the class includes corresponding aglycones, which also have potent cardiac effects. Thus, the aglycone of digitoxin, i.e., digitoxigenin, is considered to be a cardiac glycoside, even though it lacks a glycosidic moiety.) They bind to and inhibit Na⁺/K⁺-ATPase on the plasma membrane thereby leading to the increase of intracellular Ca²⁺ concentration and enhanced the cardiac contractility. The binding site has been determined to be at the extracellular side of the α-subunit of the enzyme. It has been suggested that binding of cardiac glycosides to the ATPases paralyzes the enzyme's extracellular domain and therefore affects the catalytic activity of the enzyme and ion transport. Recent studies have demonstrated another role for Na⁺/K⁺-ATPase as signal transducer ((Xie and Askari, 2002); (Aizman and Aperia, 2003), (Kometiani et al., 2005)). Binding of cardiac glycosides to the ATPase elicits interaction of the ATPase with neighboring membrane proteins leading to organized cytosolic cascades of signaling proteins to send messages to the intracellular organelles. It has also been shown that binding of ouabain, a member of cardiac glycosides, induces endocytosis of the Na⁺/K⁺-ATPase via a caveolin- and clathrin-dependent mechanism ((Liu et al., 2004); (Liu et al., 2005)). Interestingly, members of the family of cardiac glycosides possess different binding affinities and inhibitory effects to Na⁺/K⁺-ATPase ((Paula et al., 2005)).

In particular embodiments, the cardiac glycoside is

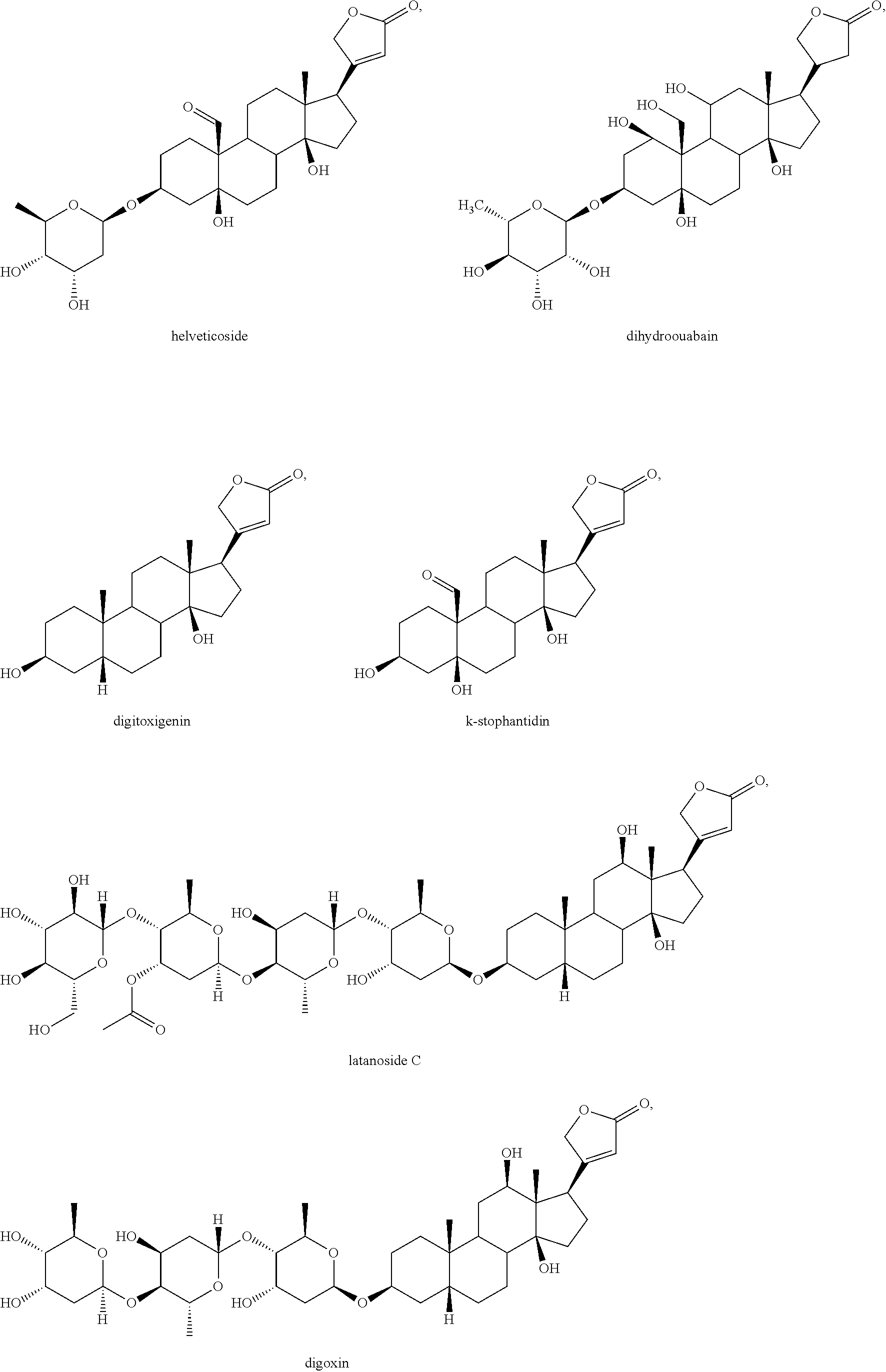

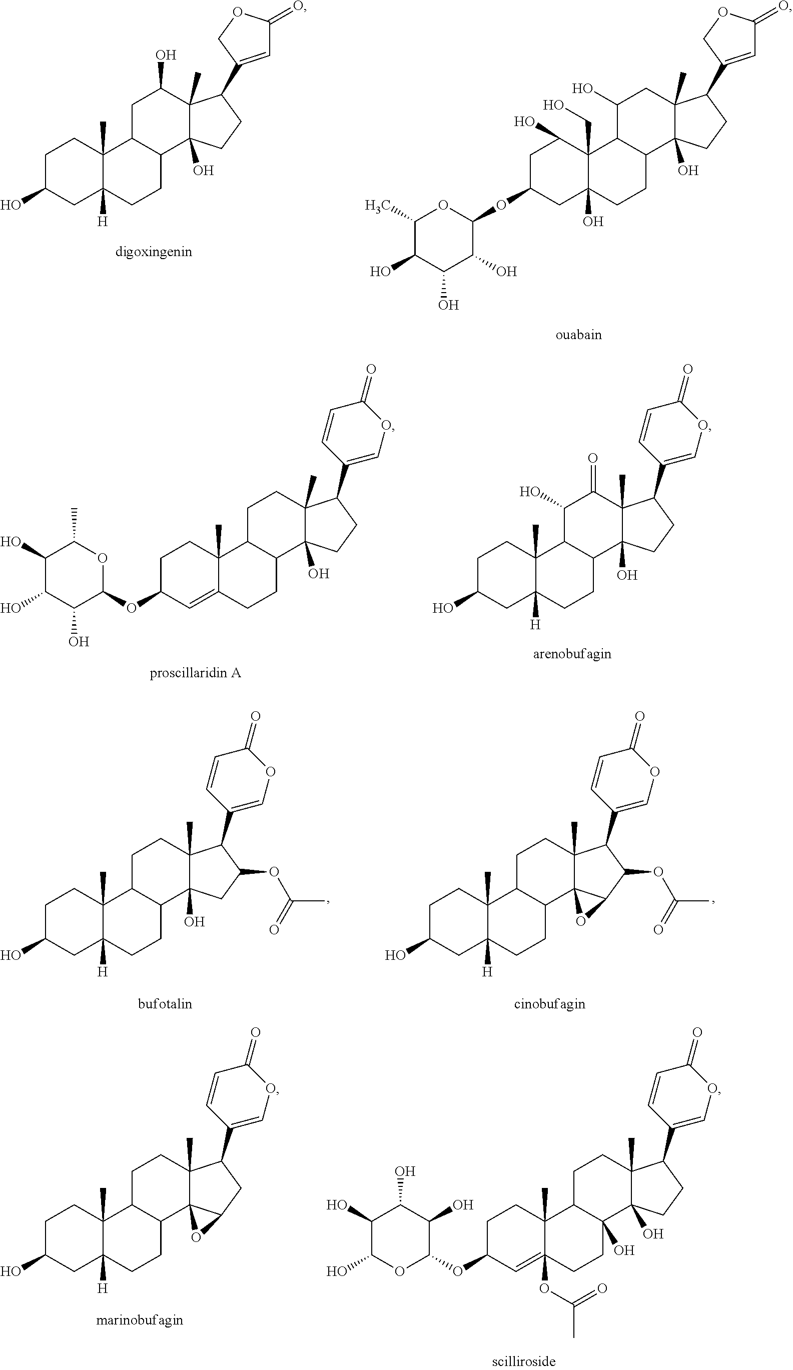

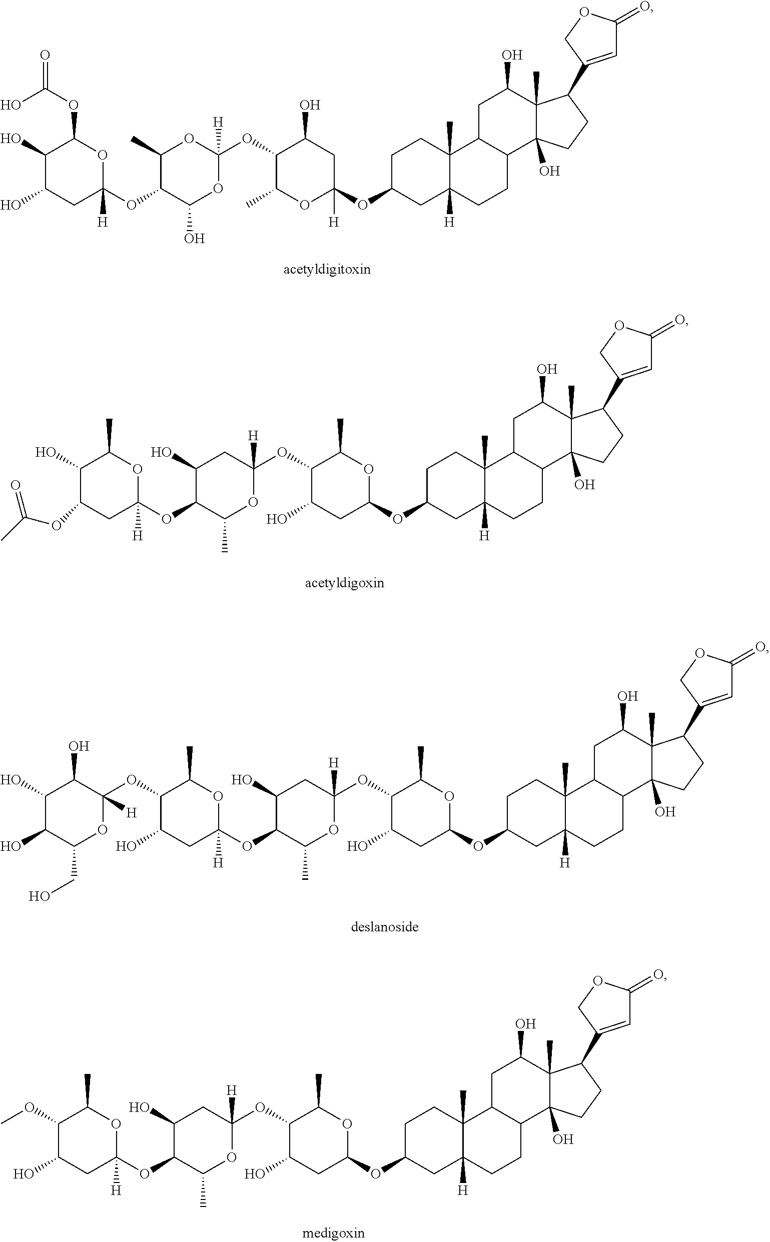

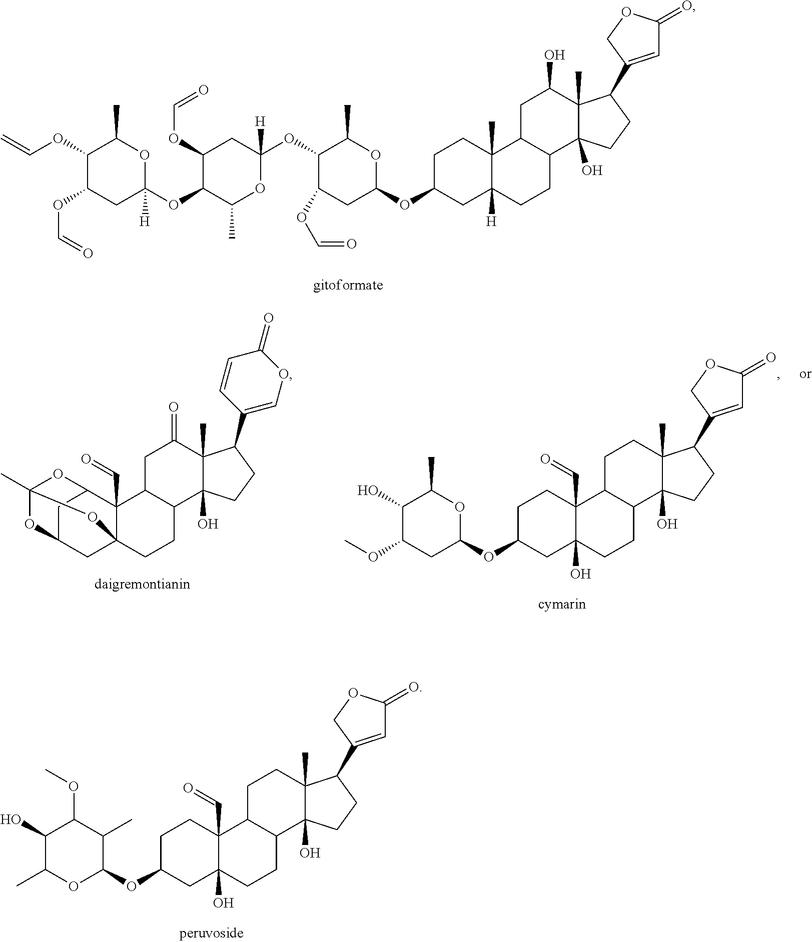

In general, a cardiac glycoside can have the structure:

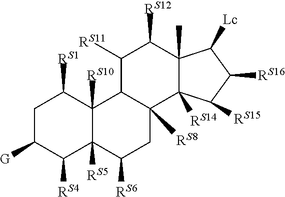

where
G is —OH or a glycoside;

### R^{S1} is —H or —OH;

R^{S4} is —H or R^{S4} and R^{S5} together form a double bond;
R^{S5} is —H, —OH, or R^{S4} and R^{S5} together form a double bond;

### R^{S6} is —H, —OH, or —OC(O)CH₃;

### R^{S8} is —H or —OH;

### R^{S10} is —CH₃, —CH₂OH, or —CHO;

### R^{S11} is —H, or —OH;

### R^{S12} is —H, —OH, or ═O;

R^{S14} is H, OH, or R^{S14} and R^{S15} taken with the atoms to which they are attached form an epoxide;
R^{S15} is H, OH, or R^{S14} and R^{S15} taken with the atoms to which they are attached form an epoxide;

### R^{S16} is H, OH, or —OC(O)CH₃; and

Lc is a lactone.
In some cases, Lc can be

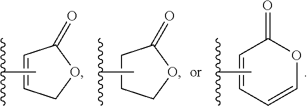

### Lipid Particles Comprising Conjugated Compounds

The term “drug-like compound” or “drug-like moiety” is well known to those skilled in the art, and may include the meaning of a compound that has characteristics that may make it suitable for use in medicine, for example as the active ingredient in a medicament. Thus, for example, a drug-like compound or moiety may be a molecule or moiety that may be synthesized by the techniques of organic chemistry, less preferably by techniques of molecular biology or biochemistry, and is preferably a small molecule, which may be of less than 5000 daltons. A drug-like compound or moiety may additionally exhibit features of selective interaction with a particular protein or proteins and be bioavailable and/or able to penetrate cellular membranes, but it will be appreciated that these features are not essential. A drug-like compound or moiety can have features including: (1) molecular mass less than 500 Daltons, (2) log P (hydrophobicity index (octanol/water partition coefficient) less than 5, (3) less than 5H-bond donors, (4) less than 10H-bond acceptors, and (5) less than 10 rotatable bonds.

As described above, methods of the present invention may be practiced using a lipid particle comprising a modified lipid comprising a compound identified herein as enhancing the uptake or cytosolic delivery of lipid particles and/or encapsulated agents, such as nucleic acids, or a functional domain or derivative thereof. Accordingly, the present invention includes modified lipids comprising levodopa, naphazoline hydrochloride, acetohexamide, niclosamide, diprophylline, isoxicam, 8-azaguanine, isoflupredone acetate, chloroquine, trimethobenzamide hydrochloride, isoxsuprine hydrochloride, or diphemanil methylsulfate, or a functional domain or derivative thereof. In related embodiments, the lipid comprises an endosomal release agent, e.g., conjugated to a lipid headgroup. In certain embodiments, the lipid is modified such that the compound is located on a region of the lipid that is exposed on the exterior of a lipid particle comprising the lipid. In particular embodiments, the modified lipid comprises the compound, or functional domain or derivative thereof at its headgroup.

In certain embodiments, the modified lipid comprises a chloroquine headgroup. Chloroquine is known to destabilize the endosomal membrane and inhibit the acidification of endosomal/lysosomal compartments, and has been used to improve gene delivery (Farhood et al., 1995; Guy et al., 1995; Budker et al., 1996). In one embodiment, such a lipid has the following structure (I):

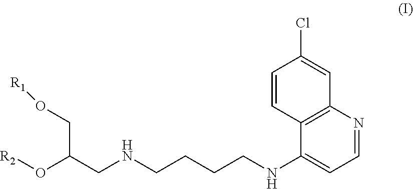

wherein R₁ and R₂ are each, independently, C₆-C₃₂ alkyl.

As used herein, “alkyl” refers to a straight or branched hydrocarbon chain radical consisting solely of carbon and hydrogen atoms, which is saturated or unsaturated (i.e., contains one or more double and/or triple bonds), having from six to thirty-two carbon atoms (C₆-C₃₂ alkyl), preferably eight to twenty-four carbon atoms (C₈-C₂₄ alkyl), and more preferably eight to twenty carbon atoms (C₈-C₂₀alkyl) and which is attached to the rest of the molecule by a single bond.

In one particular embodiment, the lipid has the following structure:

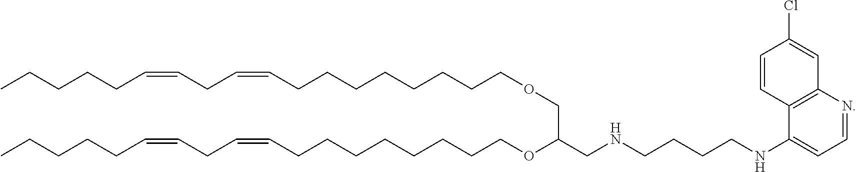

In particular embodiments, lipid particles include a compound that binds a Na+/K+-ATPase, such as a cardiac glycoside. In certain embodiments, this compound may be associated with or bound to an interior or exterior surface of the lipid particle, or it may be encapsulated within the lipid particle. In particular embodiments, this compound is conjugated to a lipid component of the lipid particle, e.g., such that the compound is exposed or presented on the exterior to the lipid particle and can, this, bind to a Na+/K+-ATPase, thereby inducing or enhancing uptake of the lipid particle and any encapsulated agent by a cell expressing the Na+/K+-ATPase.

In various embodiments, the compound that binds to a Na+/K+-ATPase is conjugated to any lipid component of the lipid particle, e.g., a cationic lipid, a non-cationic lipid, or a conjugated lipid. In certain embodiments, the compound that binds to a Na+/K+-ATPase is conjugated to a PEG-lipid. In particular embodiments, the lipid is a phospholipid or a PEG-functionalized phospholipid, such as, e.g., DSPE-PEG. Methods for conjugating small molecules such as cardiac glycosides to lipid are known and available in the art. Standard methods for coupling compounds to lipid can be used. These methods generally involve incorporation into liposomes lipid components, e.g., phosphatidylethanolamine, which can be activated for attachment of targeting agents, or derivatized lipophilic compounds, such as lipid derivatized bleomycin. In addition, conventional peptide coupling methods may be employed, and according to methods shown in the accompanying Examples.

A conjugated lipid can have the formula:

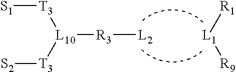

where

S₁ can be a drug-like moiety, and S₂ can be S₁ or R₅.

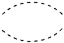

represents a connection between L₂ and L₁ which is:

(1) a single bond between one atom of L₂ and one atom of L₁, where
L₁ is C(Rₐ), O, S or N(Q);
L₂ is —(CR₅R₆)ₓ—, —C(O)—(CR₅R₆)ₓ—, —(CR₅R₆)ₓ—CR₅═CR₅—(CR₅R₆)_{y}—, —C(O)—(CR₅R₆)ₓ—CR₅═CR₅—(CR₅R₆)_{y}—, —O—, —S—, —N(Q)-, ═N—, ═C(R₅)—, —CR₅R₆—O—, —CR₅R₆—N(Q)-, —CR₅R₆—S—, —C(O)N(Q)-, —C(O)O—, —N(Q)C(O)—, —OC(O)—, —C(O)—, or —X—C(R₅)(YR₃)—;
   where X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—; and Rₐ is H, alkyl, alkoxy, —OH, —N(Q)Q, or —SQ; or

(2) a double bond between one atom of L₂ and one atom of L₁, wherein
L₁ is C;
L₂ is —(CR₅R₆)ₓ—CR₅═, —C(O)—(CR₅R₆)ₓ—CR₅═, —N(Q)═, —N—, —O—N═, —N(Q)—N═, or —C(O)N(Q)-N═;

(3) a single bond between a first atom of L₂ and a first atom of L₁, and a single bond between a second atom of L₂ and the first atom of L₁, wherein
L₁ is C or C(Rₐ)—(CR₅R₆)ₓ—C(Rₐ);
L₂ has the formula

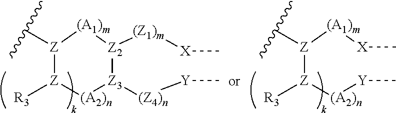

where
   X is the first atom of L₂, Y is the second atom of L₂, - - - - - represents a single bond to the first atom of L₁, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
   Z₁ and Z₄ are each, independently, —O—, —S—, —CH₂—, —CHR⁵—, or —CR⁵R⁵—; Z₂ is CH or N; Z₃ is CH or N; or Z₂ and Z₃, taken together, are a single C atom;
   A₁ and A₂ are each, independently, —O—, —S—, —CH₂—, —CHR⁵—, or —CR⁵R⁵—;
   each Z is N, C(R⁵), or C(R₃);
   k is 0, 1, or 2;
   each m, independently, is 0 to 5;
   each n, independently, is 0 to 5;
   where m and n taken together result in a 3, 4, 5, 6, 7 or 8 member ring;

(4) a single bond between a first atom of L₂ and a first atom of L₁, and a single bond between the first atom of L₂ and a second atom of L₁, where
(A) L₁ has the formula:

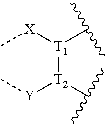

where X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
   T₁ is CH or N; T₂ is CH or N; or T₁ and T₂ taken together are C═C;
   L₂ is CR₅; or
(B) L₁ has the formula:

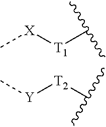

where X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
   T₁ is —CR₅R₆—, —N(Q)-, —O—, or —S—; T₂ is —CR₅R₆—, —N(Q)-, —O—, or —S—;
   L₂ is CR₅ or N;

each of x and y, independently, is 0, 1, 2, 3, 4, or 5.

R₃ can have the formula:

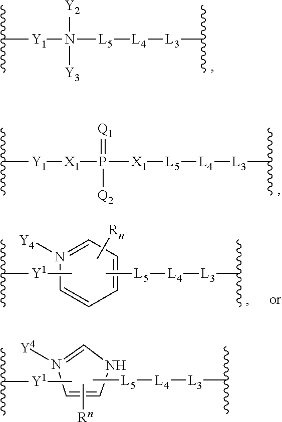

where
Q₁ is O or S;
Y₁ is a bond, alkylene, cycloalkylene, arylene, aralkylene, or alkynylene, wherein Y₁ is optionally substituted by 0 to 6 Rₙ;
Y₂ is alkyl, cycloalkyl, aryl, aralkyl, or alkynyl, wherein Y₂ is optionally substituted by 0 to 6 Rₙ;
Y₃ is absent, or if present, is alkyl, cycloalkyl, aryl, aralkyl, or alkynyl, wherein Y₃ is optionally substituted by 0 to 6 Rₙ;
Y₄ is absent, or if present, is alkyl, cycloalkyl, aryl, aralkyl, or alkynyl, wherein Y₄ is optionally substituted by 0 to 6 Rₙ; or
any two of Y₁, Y₂, and Y₃ are taken together with the N atom to which they are attached to form a 3- to 8-member heterocycle optionally substituted by 0 to 6 Rₙ; or
Y₁, Y₂, and Y₃ are all be taken together with the N atom to which they are attached to form a bicyclic 5- to 12-member heterocycle optionally substituted by 0 to 6 Rₙ;
each Rₙ, independently, is H, halo, cyano, hydroxy, amino, alkyl, alkoxy, cycloalkyl, aryl, heteroaryl, or heterocyclyl;
each X₁, independently, is —O—, —S—, or —(CR⁵R⁶)—;
L₃ is a bond, —N(Q)-, —O—, —S—, —(CR₇R₈)ₐ—, —[O—(CR₅R₆)ₐ]_{c}—, —C(O)—, or a combination of any two of these;
L₄ is a bond, —N(Q)-, —O—, —S—, —(CR₇R₈)ₐ—, —[O—(CR₅R₆)ₐ]_{c}—, —C(O)—, or a combination of any two of these;
L₅ is a bond, —N(Q)-, —O—, —S—, —(CR₇R₈)ₐ—, —[O—(CR₅R₆)ₐ]_{c}—, —C(O)—, or a combination of any two of these;
each occurrence of R₇ and R₈ is, independently, H, halo, cyano, hydroxy, amino, alkyl, alkoxy, cycloalkyl, aryl, heteroaryl, or heterocyclyl;
or two R₇ groups on adjacent carbon atoms are taken together to form a double bond between their respective carbon atoms;
or two R₇ groups on adjacent carbon atoms and two R₈ groups on the same adjacent carbon atoms are taken together to form a triple bond between their respective carbon atoms;
each a, independently, is 0, 1, 2, or 3; wherein
   an R₇ or R₈ substituent from any of L₃, L₄, or L₅ is optionally taken with an R₇ or R₈ substituent from any of L₃, L₄, or L₅ to form a 3- to 8-member cycloalkyl, heterocyclyl, aryl, or heteroaryl group;
any one of Y₁, Y₂, or Y₃, is optionally taken together with an R₇ or R₈ group from any of L₃, L₄, and L₅, and atoms to which they are attached, to form a 3- to 8-member heterocyclyl group;
each c, independently, is 0 to 2000.

### L₁₀ can be —C(R₅)— or N.

Each T₃, independently, can be a bond or -L₆-(CR₅R₆)ₘ-L₇-[(CR₅R₆)ₚO]_{q}-L₈-(CR₅R₆)ₙ-L₉- where

L₆ is a bond, —CR^{1a}R^{1b}—, —O—, —CO—, —NR^{1d}—, —S—, —P(=Q₁)(Q₂)-, or a combination thereof;

L₇ is a bond, —CR^{1a}R^{1b}—, —O—, —CO—, —NR^{1d}—, —S—, —P(=Q₁)(Q₂)-, or a combination thereof;

L₈ is a bond, —CR^{1a}R^{1b}—, —O—, —CO—, —NR^{1d}—, —S—, —P(=Q₁)(Q₂)-, or a combination thereof;

L₉ is a bond, —CR^{1a}R^{1b}—, —O—, —CO—, —NR^{1d}—, —S—, —P(=Q₁)(Q₂)-, or a combination thereof;

m is 0 to 10;

n is 0 to 10;

p is 1 to 6;

q is 0 to 2000.

Each occurrence of R₅ and R₆ can be, independently, H, halo, cyano, hydroxy, amino, alkyl, alkoxy, cycloalkyl, aryl, heteroaryl, or heterocyclyl.

each Q, independently, is H, alkyl, acyl, cycloalkyl, alkenyl, alkynyl, aryl, heteroaryl or heterocyclyl.

Each Q₂, independently, can be —OQ, —SQ, —N(Q)Q, alkyl, or alkoxy. R¹ is a C₁₀ to C₃₀ group having the formula

-L^{1a}-(CR^{1a}R^{1b})_{α}-[L^{1b}-(CR^{1a}R^{1b})_{β}]_{γ}-L^{1c}-R^{1c},

where
L^{1a} is a bond, —CR^{1a}R^{1b}—, —O—, —CO—, —NR^{1d}—, —S—, or a combination thereof; each R^{1a} and each R^{1b}, independently, is H; halo; hydroxy; cyano; C₁-C₆ alkyl optionally substituted by halo, hydroxy, or alkoxy; C₃-C₈ cycloalkyl optionally substituted by halo, hydroxy, or alkoxy; —OR^{1c}; —NR^{1c}R^{1d}; aryl; heteroaryl; or heterocyclyl;
each L^{1b}, independently, is a bond, —(CR^{1a}R^{1b})₁₋₂—, —O—, —CO—, —NR^{1d}—, —S—,

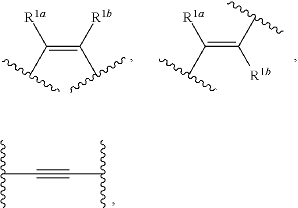

or a combination thereof; or has the formula

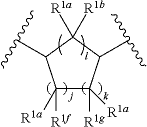

wherein j, k, and 1 are each independently 0, 1, 2, or 3, provided that the sum of j, k and 1 is at least 1 and no greater than 8; and R^{1f} and R^{1g} are each independently R^{1b}, or adjacent R^{1f} and R^{1g}, taken together, are optionally a bond;
or has the formula

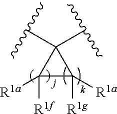

wherein j and k are each independently 0, 1, 2, 3, or 4 provided that the sum of j and k is at least 1; and R^{1f} and R^{1g} are each independently R^{1b}, or adjacent R^{1f} and R^{1g}, taken together, are optionally a bond;
or has the formula:

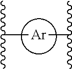

where —Ar— is a 6 to 14 membered arylene group optionally substituted by zero to six R^{1a} groups;
or has the formula:

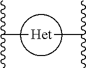

where -Het- is a 3 to 14 membered heterocyclylene or heteroarylene group optionally substituted by zero to six R^{1a} groups;
L^{1c} is —(CR^{1a}R^{1b})₁₋₂—, —O—, —CO—, —NR^{1d}—, —S—,

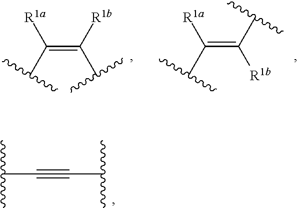

or a combination thereof;
R^{1c} is H; halo; hydroxy; cyano; C₁-C₆ alkyl optionally substituted by halo, hydroxy, or alkoxy; C₃-C₈ cycloalkyl optionally substituted by halo, hydroxy, or alkoxy; aryl; heteroaryl; or heterocyclyl; or R^{1c} has the formula:

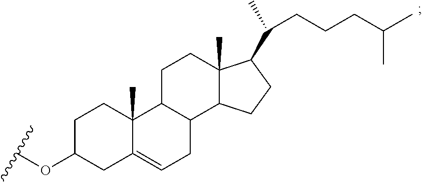

R^{1d} is H; halo; hydroxy; cyano; C₁-C₆ alkyl optionally substituted by halo, hydroxy, or alkoxy; C₃-C₈ cycloalkyl optionally substituted by halo, hydroxy, or alkoxy; aryl; heteroaryl; or heterocyclyl;
α is 0-6;
each β, independently, is 0-6; and
γ is 0-6.

### R⁹ can be R² or R⁵.

R² can be a C₁₀ to C₃₀ group having the formula

-L^{2a}-(CR^{2a}R^{2b})_{δ}-[L^{2b}(CR^{2a}R^{2b})_{ε}]_{ζ}-L^{2c}-R^{2c},
wherein L^{2a} is a bond, —CR^{2a}R^{2b}—, —O—, —CO—, —NR^{2d}—, —S—, or a combination thereof;
each R^{2a} and each R^{2b}, independently, is H; halo; hydroxy; cyano; C₁-C₆ alkyl optionally substituted by halo, hydroxy, or alkoxy; C₃-C₈ cycloalkyl optionally substituted by halo, hydroxy, or alkoxy; —OR^{1c}; —NR^{2c}R^{2d}; aryl; heteroaryl; or heterocyclyl;
each L^{2b}, independently, is a bond, —(CR^{2a}R^{2b})₁₋₂—, —O—, —CO—, —NR^{2d}—, —S—,

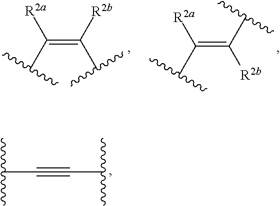

or a combination thereof; or has the formula:

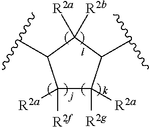

wherein j, k, and 1 are each independently 0, 1, 2, or 3, provided that the sum of j, k and 1 is at least 1 and no greater than 8; and R^{2f} and R^{2g} are each independently R^{2b}, or adjacent R^{2f} and R^{2g}, taken together, are optionally a bond;
or has the formula:

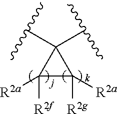

wherein j and k are each independently 0, 1, 2, 3, or 4 provided that the sum of j and k is at least 1; and R^{2f} and R^{2g} are each independently R^{2b}, or adjacent R^{2f} and R^{2g}, taken together, are optionally a bond;
or has the formula:

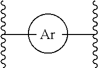

wherein —Ar— is a 6 to 14 membered arylene group optionally substituted by zero to six R^{2a} groups
or has the formula:

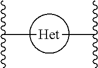

wherein -Het- is a 3 to 14 membered heterocyclylene or heteroarylene group optionally substituted by zero to six R^{2a} groups;
L^{2c} is —(CR^{2a}R^{2b})₁₋₂—, —O—, —CO—, —NR^{2d}—, —S—,

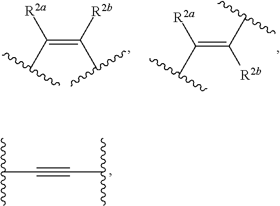

or a combination thereof;
R^{2c} is H; halo; hydroxy; cyano; C₁-C₆ alkyl optionally substituted by halo, hydroxy, or alkoxy; C₃-C₈ cycloalkyl optionally substituted by halo, hydroxy, or alkoxy; aryl; heteroaryl; or heterocyclyl; or R^{2c} has the formula:

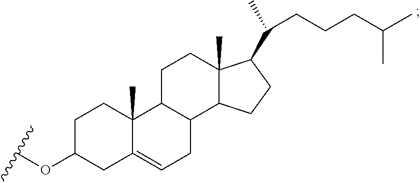

R^{2d} is H; halo; hydroxy; cyano; C₁-C₆ alkyl optionally substituted by halo, hydroxy, or alkoxy; C₃-C₈cycloalkyl optionally substituted by halo, hydroxy, or alkoxy; aryl; heteroaryl; or heterocyclyl;
δ is 0-6;
each ε, independently, is 0-6; and
ζ is 0-6.
   The lipid can be in the form of a pharmaceutically acceptable salt.

### Methods of Enhancing Uptake and Cytosolic Distribution of Lipid Particles and Encapsulated Agents and Methods of Treating or Preventing Diseases and Disorders

As described in the accompanying Examples, compounds capable of enhancing the cellular uptake or cytosolic distribution of a lipid particle and/or its encapsulated nucleic acid were identified. Accordingly, these compounds may be used in combination with lipid particles to enhance the cellular uptake or cytosolic delivery of agents encapsulated in lipid particles. Thus, methods are described that may be used to enhance delivery or cytosolic distribution of a therapeutic agent, such as a siRNA, to a cell, for the treatment or prevention of a disease or disorder.

A method of enhancing cellular uptake of a lipid particle includes contacting a cell with a lipid particle and a compound selected from levodopa, naphazoline hydrochloride, acetohexamide, niclosamide, diprophylline, and isoxicam, or a combination thereof. The lipid particle can include a therapeutic agent, and the method may be used to enhance cellular uptake of the encapsulated therapeutic agent, e.g., an interfering RNA such as an siRNA.

A method for enhancing cytosolic distribution of a lipid particle includes contacting a cell with a compound selected from azaguanine-8, isoflupredone acetate, chloroquine, trimethobenzamide, hydrochloride, isoxsuprine hydrochloride, and diphemanil methylsulfate, or a combination thereof; and a lipid particle. The lipid particle can include a therapeutic agent, and the method may be used to enhance cytosolic distribution of the encapsulated therapeutic agent, e.g., an interfering RNA such as an siRNA.

A method of treating or preventing a disease or disorder in a subject includes administering to the subject a compound selected from levodopa, naphazoline hydrochloride, acetohexamide, niclosamide, diprophylline, isoxicam, azaguanine-8, isoflupredone acetate, chloroquine, trimethobenzamide, hydrochloride, isoxsuprine hydrochloride, and diphemanil methylsulfate, or a combination thereof; and a lipid particle comprising a therapeutic agent, where the therapeutic agent is effective in treating or preventing said disease or disorder. The cell can be a mammalian cell, e.g., a human cell. The disease or disorder can be a tumor, an inflammatory disease or disorder, a metabolic disease or disorder, a neurological disease or disorder, or a cardiac disease or disorder.

The methods may also be practiced using lipid particles comprising lipids modified to include a compound described above or a functional domain or derivative thereof. For example, lipid particles comprising one of more lipids modified to include a headgroup including levodopa, naphazoline hydrochloride, acetohexamide, niclosamidediprophylline, isoxicam, azaguanine-8, isoflupredone acetate, chloroquine, trimethobenzamide, hydrochloride, isoxsuprine hydrochloride, or diphemanil methylsulfate, or a functional domain or derivative thereof, may be used to enhance uptake or cytosolic distribution of the lipid particle and/or an encapsulated agent. The methods can be practiced using lipid particles comprising a lipid including a chloroquine headgroup, including those described herein, e.g., a lipid having the following structure (I):

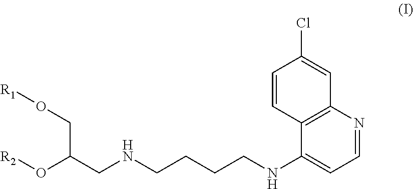

wherein R₁ and R₂ are each, independently, C₆-C₃₂alkyl, such as a lipid having the following structure:

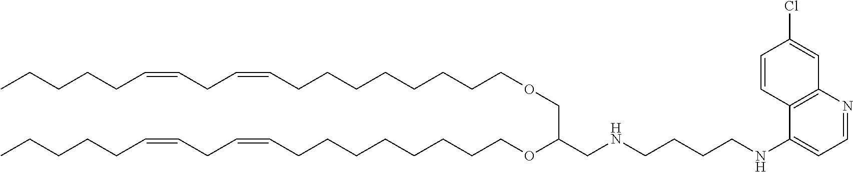

Various embodiments of the present invention may be practiced to enhance the cellular uptake or cytosolic distribution of a lipid particle, e.g., a lipid particle comprising a therapeutic agent. The present methods may be used to deliver an encapsulated agent to a variety of different cells and subcellular locations. Accordingly, the methods of the invention may be used to modulate the expression of a variety of different genes, modulate an immune response, and treat or prevent various related diseases and disorders.

### Methods of Enhancing Uptake of Lipid Particles and Treating or Preventing Diseases and Disorders

A method of enhancing cellular uptake of a lipid particle can include contacting a cell with a lipid particle and a compound that binds a Na+/K+-ATPase. Contacting can occurs in vitro or in vivo. The cell can be a mammalian cell, e.g., a human cell. The lipid particle can include a therapeutic agent.

A method of treating or preventing a disease or disorder in a subject can involve providing to the subject a compound that binds a Na+/K+-ATPase and a lipid particle comprising a therapeutic agent. The subject can be a mammal, e.g., a human. The disease or disorder can be a tumor, an inflammatory disease or disorder, a metabolic disease or disorder, a neurological disease or disorder, or a cardiac disease or disorder.

The lipid particle and the compound that binds a Na+/K+-ATPase may be contacted with a cell or provided to a subject at the same time. However, the compound and the lipid particle may be delivered via different routes of administration and one may be contacted or delivered before or after the other.

There can be various ways to enhance the cellular uptake or delivery of a lipid particle, e.g., a lipid particle comprising a therapeutic agent to the interior of a cell. The present methods may be used to deliver an encapsulated agent to a variety of different cells and subcellular locations. Accordingly, the methods may be used to modulate the expression of a variety of different genes, modulate an immune response, and treat or prevent various related diseases and disorders, including inflammatory or immune-related diseases and disorders.

The methods may be carried out in vitro or in vivo, and include methods for enhancing the introduction of a lipid particle including a nucleic acid, e.g., an interfering RNA, into a cell. Preferred nucleic acids for introduction into cells are siRNA. These methods may be carried out by contacting lipid particles including nucleic acids according to methods for a period of time sufficient for intracellular delivery to occur.

Contact between the cells and the lipid particles, when carried out in vitro, may take place in a biologically compatible medium. The concentration of lipid particles in the medium can vary widely depending on the particular application, but is generally between about 1 mol and about 10 mmol. In certain embodiments, treatment of the cells with the lipid particles will generally be carried out at physiological temperatures (about 37° C.) for periods of time from about 1 to 24 hours, preferably from about 2 to 8 hours. For in vitro applications, the cell may be grown or maintained in culture, whether of plant or animal origin, vertebrate or invertebrate, and of any tissue or type. In preferred embodiments, the cells will be animal cells, more preferably mammalian cells, and most preferably human cells.

Typical applications of the methods and compositions include enhancing intracellular delivery of siRNA to knock down or silence specific cellular targets.

For in vivo administration, pharmaceutical compositions comprising lipid particles may be administered by any means available in the art. For example, they may be administered parenterally, i.e., intraarticularly, intravenously, intraperitoneally, subcutaneously, or intramuscularly. In particular embodiments, the pharmaceutical compositions are administered intravenously or intraperitoneally by a bolus injection. For one example, see Stadler, et al., U.S. Pat. No. 5,286,634, which is incorporated herein by reference. Intracellular nucleic acid delivery has also been discussed in Straubringer, et al., METHODS IN ENZYMOLOGY, Academic Press, New York. 101:512-527 (1983); Mannino, et al., Biotechniques 6:682-690 (1988); Nicolau, et al., Crit. Rev. Ther. Drug Carrier Syst. 6:239-271 (1989), and Behr, Acc. Chem. Res. 26:274-278 (1993), each of which is incorporated by reference in its entirety. Still other methods of administering lipid-based therapeutics are described in, for example, Rahman et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578, each of which is incorporated by reference in its entirety. In other methods, the pharmaceutical preparations may be contacted with a desired tissue by direct application of the preparation to the tissue. The lipid particles can also be administered in an aerosol inhaled into the lungs (see, Brigham, et al., Am. J. Sci. 298(4):278-281 (1989), which is incorporated by reference in its entirety) or by direct injection at the site of disease (Culver, Human Gene Therapy, MaryAnn Liebert, Inc., Publishers, New York. pp. 70-71 (1994), which is incorporated by reference in its entirety).

The methods may be practiced in a variety of subjects or hosts. Preferred subjects or hosts include mammalian species, such as humans, non-human primates, dogs, cats, cattle, horses, sheep, and the like. In particular embodiments, the subject is a mammal, such as a human, in need of treatment or prevention of a disease or disorder, e.g., a subject diagnosed with or considered at risk for a disease or disorder.

Dosages for the lipid particles of the present invention will depend on the ratio of nucleic acid to lipid and the administrating physician's opinion based on age, weight, and condition of the patient.

### Lipid Particles

While any agent, e.g., antibodies, polypeptides, toxins, or small molecules, may be delivered to a cell or tissue according to the methods, in particular embodiments, methods are practiced using lipid particles including a nucleic acid. The nucleic acid may include DNA, RNA, or both, including modified forms of DNA and/or RNA. In certain embodiments, the nucleic acid is single-stranded or double-stranded.

The lipid particle can include an interfering RNA capable of mediating knockdown (i.e., reduced expression) of a target gene. In particular embodiments, the particles are stable nucleic acid-lipid particles (SNALPs). A SNALP represents a particle made from lipids (e.g., a cationic lipid, a non-cationic lipid and a conjugated lipid that prevents aggregation of the particle), where the nucleic acid (e.g., siRNA, microRNA (miRNA), short hairpin RNA (shRNA), including plasmids from which an interfering RNA is transcribed) is encapsulated within the lipid.

As used herein, “lipid encapsulated” refers to a lipid formulation that provides a compound, such as a nucleic acid (e.g., a siRNA), with full encapsulation, partial encapsulation, or both. In a preferred embodiment, the nucleic acid is fully encapsulated in the lipid formulation (e.g., to form an SPLP, pSPLP, SNALP, or other nucleic acid-lipid particle). In both instances, the nucleic acid is protected from nuclease degradation.

### RNA Interference Nucleic Acids

In particular embodiments, nucleic acid-lipid particles are associated with RNA interference (RNAi) molecules. RNA interference methods using RNAi molecules may be used to disrupt the expression of a gene or polynucleotide of interest. Small interfering RNA (siRNA) has essentially replaced antisense ODN and ribozymes as the next generation of targeted oligonucleotide drugs under development.

SiRNAs are RNA duplexes normally 16-30 nucleotides long that can associate with a cytoplasmic multi-protein complex known as RNAi-induced silencing complex (RISC). RISC loaded with siRNA mediates the degradation of homologous mRNA transcripts, therefore siRNA can be designed to knock down protein expression with high specificity. Unlike other antisense technologies, siRNA function through a natural mechanism evolved to control gene expression through non-coding RNA. This is generally considered to be the reason why their activity is more potent in vitro and in vivo than either antisense ODN or ribozymes. A variety of RNAi reagents, including siRNAs targeting clinically relevant targets, are currently under pharmaceutical development, as described, e.g., in de Fougerolles, A. et al., Nature Reviews 6:443-453 (2007), which is incorporated by reference in its entirety.

While the first described RNAi molecules were RNA:RNA hybrids comprising both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., (2003) Molecular Biotechnology 24:111-119). Thus, the use of RNAi molecules comprising any of these different types of double-stranded molecules is contemplated. In addition, it is understood that RNAi molecules may be used and introduced to cells in a variety of forms. Accordingly, as used herein, RNAi molecules encompasses any and all molecules capable of inducing an RNAi response in cells, including, but not limited to, double-stranded oligonucleotides comprising two separate strands, i.e. a sense strand and an antisense strand, e.g., small interfering RNA (siRNA); double-stranded oligonucleotide comprising two separate strands that are linked together by non-nucleotidyl linker; oligonucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule

A single strand siRNA compound may be sufficiently long that it can enter the RISC and participate in RISC mediated cleavage of a target mRNA. A single strand siRNA compound is at least 14, and in other embodiments at least 15, 20, 25, 29, 35, 40, or 50 nucleotides in length. In certain embodiments, it is less than 200, 100, or 60 nucleotides in length.

Hairpin siRNA compounds will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region. In certain embodiments, the overhangs are 2-3 nucleotides in length. In some embodiments, the overhang is at the sense side of the hairpin and in some embodiments on the antisense side of the hairpin.

A “double stranded siRNA compound” as used herein, is an siRNA compound which includes more than one, and in some cases two, strands in which interchain hybridization can form a region of duplex structure.

The antisense strand of a double stranded siRNA compound may be equal to or at least, 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length. As used herein, term “antisense strand” means the strand of an siRNA compound that is sufficiently complementary to a target molecule, e.g. a target RNA.

The sense strand of a double stranded siRNA compound may be equal to or at least 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The double strand portion of a double stranded siRNA compound may be equal to or at least, 14, 15, 16 17, 18, 19, 20, 21, 22, 23, 24, 25, 29, 40, or 60 nucleotide pairs in length. It may be equal to or less than 200, 100, or 50, nucleotides pairs in length. Ranges may be 15-30, 17 to 23, 19 to 23, and 19 to 21 nucleotides pairs in length.

In many embodiments, the siRNA compound is sufficiently large that it can be cleaved by an endogenous molecule, e.g., by Dicer, to produce smaller siRNA compounds, e.g., siRNAs agents

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1-3 nucleotides. The overhangs can be the result of one strand being longer than the other, or the result of two strands of the same length being staggered. Some embodiments will have at least one 3′ overhang. In one embodiment, both ends of an siRNA molecule will have a 3′ overhang. In some embodiments, the overhang is 2 nucleotides.

In certain embodiments, the length for the duplexed region is between 15 and 30, or 18, 19, 20, 21, 22, and 23 nucleotides in length, e.g., in the ssiRNA compound range discussed above. ssiRNA compounds can resemble in length and structure the natural Dicer processed products from long dsiRNAs. Embodiments in which the two strands of the ssiRNA compound are linked, e.g., covalently linked are also included. Hairpin, or other single strand structures which provide the required double stranded region, and a 3′ overhang are also contemplated.

The siRNA compounds described herein, including double-stranded siRNA compounds and single-stranded siRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an ssiRNA compound of 21 to 23 nucleotides.

In one embodiment, an siRNA compound is “sufficiently complementary” to a target RNA, e.g., a target mRNA, such that the siRNA compound silences production of protein encoded by the target mRNA. In another embodiment, the siRNA compound is “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

### MicroRNAs

Micro RNAs (miRNAs) are a highly conserved class of small RNA molecules that are transcribed from DNA in the genomes of plants and animals, but are not translated into protein. Processed miRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S, NAR, 2004, 32, Database Issue, D109-D111; and also at http://microrna.sanger.ac.uk/sequences/.

### Antisense Oligonucleotides

In one embodiment, a nucleic acid is an antisense oligonucleotide directed to a target polynucleotide. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence, e.g. a target gene mRNA. Antisense oligonucleotides are thought to inhibit gene expression by binding to a complementary mRNA. Binding to the target mRNA can lead to inhibition of gene expression either by preventing translation of complementary mRNA strands by binding to it, or by leading to degradation of the target mRNA. Antisense DNA can be used to target a specific, complementary (coding or non-coding) RNA. If binding takes places this DNA/RNA hybrid can be degraded by the enzyme RNase H. In particular embodiments, antisense oligonucleotides contain from about 10 to about 50 nucleotides, more preferably about 15 to about 30 nucleotides. The term also encompasses antisense oligonucleotides that may not be exactly complementary to the desired target gene. Thus, instances where non-target specific-activities are found with antisense, or where an antisense sequence containing one or more mismatches with the target sequence is the most preferred for a particular use, are contemplated.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829 each of which is incorporated by reference). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science. 1988 Jun. 10; 240(4858):1544-6; Vasanthakumar and Ahmed, Cancer Commun. 1989; 1(4):225-32; Peris et al., Brain Res Mol Brain Res. 1998 Jun. 15; 57(2):310-20; U.S. Pat. No. 5,801,154; U.S. Pat. No. 5,789,573; U.S. Pat. No. 5,718,709 and U.S. Pat. No. 5,610,288, each of which is incorporated by reference). Furthermore, antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683, each of which is incorporated by reference).

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tₘ, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

### Antagomirs

Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety. Antagomir RNAs may be synthesized using standard solid phase oligonucleotide synthesis protocols. See U.S. Patent Application Publication Nos. 2007/0123482 and 2007/0213292 (each of which is incorporated herein by reference).

An antagomir can include ligand-conjugated monomer subunits and monomers for oligonucleotide synthesis. Exemplary monomers are described in U.S. Patent Application Publication No. 2005/0107325, which is incorporated by reference in its entirety. An antagomir can have a ZXY structure, such as is described in WO 2004/080406, which is incorporated by reference in its entirety. An antagomir can be complexed with an amphipathic moiety. Exemplary amphipathic moieties for use with oligonucleotide agents are described in WO 2004/080406, which is incorporated by reference in its entirety.

### Aptamers

Aptamers are nucleic acid or peptide molecules that bind to a particular molecule of interest with high affinity and specificity (Tuerk and Gold, Science 249:505 (1990); Ellington and Szostak, Nature 346:818 (1990), and U.S. Pat. Nos. 5,270,163 and 5,475,096, each of which is incorporated by reference in its entirety). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9 (1999), and Hermann and Patel, Science 287:820-5 (2000), each of which is incorporated by reference in its entirety. Aptamers may be RNA or DNA based, and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, as described more fully herein, the term “aptamer” specifically includes “secondary aptamers” containing a consensus sequence derived from comparing two or more known aptamers to a given target.

### Ribozymes

According to another embodiment, nucleic acid-lipid particles are associated with ribozymes. Ribozymes are RNA molecules complexes having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). For example, a large number of ribozymes accelerate phosphoester transfer reactions with a high degree of specificity, often cleaving only one of several phosphoesters in an oligonucleotide substrate (Cech et al., Cell. 1981 December; 27(3 Pt 2):487-96; Michel and Westhof, J Mol. Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis δ virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in U.S. Pat. No. 4,987,071. Important characteristics of enzymatic nucleic acid molecules used are that they have a specific substrate binding site which is complementary to one or more of the target gene DNA or RNA regions, and that they have nucleotide sequences within or surrounding that substrate binding site which impart an RNA cleaving activity to the molecule. Thus the ribozyme constructs need not be limited to specific motifs mentioned herein.

Methods of producing a ribozyme targeted to any polynucleotide sequence are known in the art. Ribozymes may be designed as described in Int. Pat. Appl. Publ. Nos. WO 93/23569 and WO 94/02595, each specifically incorporated herein by reference, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. Nos. WO 92/07065, WO 93/15187, and WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

### Immunostimulatory Oligonucleotides

Nucleic acids associated with lipid particles may be immunostimulatory, including immunostimulatory oligonucleotides (ISS; single- or double-stranded) capable of inducing an immune response when administered to a subject, which may be a mammal or other patient. ISS include, e.g., certain palindromes leading to hairpin secondary structures (see Yamamoto S., et al. (1992) J. Immunol. 148: 4072-4076, which is incorporated by reference in its entirety), or CpG motifs, as well as other known ISS features (such as multi-G domains, see WO 96/11266, which is incorporated by reference in its entirety).

The immune response may be an innate or an adaptive immune response. The immune system is divided into a more innate immune system, and acquired adaptive immune system of vertebrates, the latter of which is further divided into humoral cellular components. In particular embodiments, the immune response may be mucosal.

In particular embodiments, an immunostimulatory nucleic acid is only immunostimulatory when administered in combination with a lipid particle, and is not immunostimulatory when administered in its “free form.” Such an oligonucleotide is considered to be immunostimulatory.

Immunostimulatory nucleic acids are considered to be non-sequence specific when it is not required that they specifically bind to and reduce the expression of a target polynucleotide in order to provoke an immune response. Thus, certain immunostimulatory nucleic acids may comprise a sequence corresponding to a region of a naturally occurring gene or mRNA, but they may still be considered non-sequence specific immunostimulatory nucleic acids.

In one embodiment, the immunostimulatory nucleic acid or oligonucleotide comprises at least one CpG dinucleotide. The oligonucleotide or CpG dinucleotide may be unmethylated or methylated. In another embodiment, the immunostimulatory nucleic acid comprises at least one CpG dinucleotide having a methylated cytosine. In one embodiment, the nucleic acid comprises a single CpG dinucleotide, wherein the cytosine in said CpG dinucleotide is methylated. In a specific embodiment, the nucleic acid comprises the sequence 5′ TAACGTTGAGGGGCAT 3′. In an alternative embodiment, the nucleic acid comprises at least two CpG dinucleotides, wherein at least one cytosine in the CpG dinucleotides is methylated. In a further embodiment, each cytosine in the CpG dinucleotides present in the sequence is methylated. In another embodiment, the nucleic acid comprises a plurality of CpG dinucleotides, wherein at least one of said CpG dinucleotides comprises a methylated cytosine.

In one specific embodiment, the nucleic acid comprises the sequence 5′ TTCCATGACGTTCCTGACGT 3′. In another specific embodiment, the nucleic acid sequence comprises the sequence 5′ TCCATGACGTTCCTGACGT 3′, wherein the two cytosines indicated in bold are methylated. In particular embodiments, the ODN is selected from a group of ODNs consisting of ODN #1, ODN #2, ODN #3, ODN #4, ODN #5, ODN #6, ODN #7, ODN #8, and ODN #9, as shown below.

TABLE 5
Exemplary Immunostimulatory Oligonucleotides (ODNs)

| ODN NAME | SEQ ID | ODN SEQUENCE (5′-3′) |
|---|---|---|
| ODN 1 | | 5′-TAACGTTGAGGGGCAT-3 |
| human c-myc | | |
| * ODN 1m | | 5′-TAAZGTTGAGGGGCAT-3 |
| ODN 2 | | 5′-TCCATGACGTTCCTGACGTT-3 |
| * ODN 2m | | 5′-TCCATGAZGTTCCTGAZGTT-3 |
| ODN 3 | | 5′-TAAGCATACGGGGTGT-3 |
| ODN 5 | | 5′-AACGTT-3 |
| ODN 6 | | 5′-GATGCTGTGTCGGGGTCTCCGGGC-3′ |
| ODN 7 | | 5′-TCGTCGTTTTGTCGTTTTGTCGTT-3′ |
| ODN 7m | | 5′-TZGTZGTTTTGTZGTTTTGTZGTT-3′ |
| ODN 8 | | 5′-TCCAGGACTTCTCAGGTT-3′ |
| ODN 9 | | 5′-TCTCCCAGCGTGCGCCAT-3′ |
| ODN 10 murine Intracellular | | 5′-TGCATCCCCCAGGCCACCAT-3 |
| Adhesion Molecule-1 | | |
| ODN 11 human Intracellular | | 5′-GCCCAAGCTGGCATCCGTCA-3′ |
| Adhesion Molecule-1 | | |
| ODN 12 human Intracellular | | 5′-GCCCAAGCTGGCATCCGTCA-3′ |
| Adhesion Molecule-1 | | |
| ODN 13 human erb-B-2 | | 5′-GGT GCTCACTGC GGC-3′ |
| ODN 14 human c-myc | | 5′-AACC GTT GAG GGG CAT-3′ |
| ODN 15 human c-mys | | 5′-TAT GCT GTG CCG GGG TCT TCG GGC-3′ |
| ODN 16 | | 5′-GTGCCG GGGTCTTCGGGC-3′ |
| ODN 17 human Insulin Growth | | 5′-GGACCCTCCTCCGGAGCC-3′ |
| Factor 1-Receptor | | |
| ODN 18 human Insulin Growth | | 5′-TCC TCC GGA GCC AGA CTT-3′ |
| Factor 1-Receptor | | |
| ODN 19 human Epidermal | | 5′-AAC GTT GAG GGG CAT-3′ |
| Growth Factor-Receptor | | |
| ODN 20 Epidermal Growth | | 5′-CCGTGGTCA TGCTCC-3′ |
| Factor-Receptor | | |
| ODN 21 human Vascular | | 5′-CAG CCTGGCTCACCG CCTTGG-3′ |
| Endothelial Growth Factor | | |
| ODN 22 murine Phosphokinase | | 5′-CAG CCA TGG TTC CCC CCA AC-3′ |
| C-alpha | | |
| ODN 23 | | 5′-GTT CTC GCT GGT GAG TTT CA-3′ |
| ODN 24 human Bcl-2 | | 5′-TCT CCCAGCGTGCGCCAT-3′ |
| ODN 25 human C-Raf-s | | 5′-GTG CTC CAT TGA TGC-3′ |
| ODN #26 human Vascular | | 5′-GAGUUCUGAUGAGGCCGAAAGGCCGAAAGUCUG-3′ |
| Endothelial Growth | | |
| Factor Receptor-1 | | |
| ODN #27 | | 5′-RRCGGY-3′ |
| ODN #28 | | 5′-AACGTTGAGGGGCAT-3′ |
| ODN #29 | | 5′-CAACGTTATGGGGAGA-3′ |
| ODN #30 human c-myc | | 5′-TAACGTTGAGGGGCAT-3′ |

“Z” represents a methylated cytosine residue. ODN 14 is a 15-mer oligonucleotide and ODN 1 is the same oligonucleotide having a thymidine added onto the 5′ end making ODN 1 into a 16-mer. No difference in biological activity between ODN 14 and ODN 1 has been detected and both exhibit similar immunostimulatory activity (Mui et al., 2001)

Additional specific nucleic acid sequences of suitable oligonucleotides (ODNs) are described in Raney et al., Journal of Pharmacology and Experimental Therapeutics, 298:1185-1192 (2001), incorporated by reference in its entirety. In certain embodiments, ODNs used in the compositions and methods of the present invention have a phosphodiester (“PO”) backbone or a phosphorothioate (“PS”) backbone, and/or at least one methylated cytosine residue in a CpG motif.

### Decoy Oligonucleotides

Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor. Examples of the utilization of decoy oligonucleotides may be found in Mann et al., J. Clin. Invest., 2000, 106: 1071-1075, which is expressly incorporated by reference herein, in its entirety.

### Supermir

A supermir refers to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target. This term includes oligonucleotides composed of naturally-occurring nucleobases, sugars and covalent internucleoside (backbone) linkages and which contain at least one non-naturally-occurring portion which functions similarly. Such modified or substituted oligonucleotides are preferred over native forms because of desirable properties such as, for example, enhanced cellular uptake, enhanced affinity for nucleic acid target and increased stability in the presence of nucleases. In a preferred embodiment, the supermir does not include a sense strand, and in another preferred embodiment, the supermir does not self-hybridize to a significant extent. A supermir can have secondary structure, but it is substantially single-stranded under physiological conditions. An supermir that is substantially single-stranded is single-stranded to the extent that less than about 50% (e.g., less than about 40%, 30%, 20%, 10%, or 5%) of the supermir is duplexed with itself. The supermir can include a hairpin segment, e.g., sequence, preferably at the 3′ end can self hybridize and form a duplex region, e.g., a duplex region of at least 1, 2, 3, or 4 and preferably less than 8, 7, 6, or n nucleotides, e.g., 5 nucleotides. The duplexed region can be connected by a linker, e.g., a nucleotide linker, e.g., 3, 4, 5, or 6 dTs, e.g., modified dTs. In another embodiment the supermir is duplexed with a shorter oligo, e.g., of 5, 6, 7, 8, 9, or 10 nucleotides in length, e.g., at one or both of the 3′ and 5′ end or at one end and in the non-terminal or middle of the supermir.

miRNA Mimics

miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs). miRNA mimics can be comprised of nucleic acid (modified or modified nucleic acids) including oligonucleotides comprising, without limitation, RNA, modified RNA, DNA, modified DNA, locked nucleic acids, or 2′-O,4′-C-ethylene-bridged nucleic acids (ENA), or any combination of the above (including DNA-RNA hybrids). In addition, miRNA mimics can comprise conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can comprise 2′ modifications (including 2′-O methyl modifications and 2′ F modifications) on one or both strands of the molecule and internucleotide modifications (e.g. phorphorthioate modifications) that enhance nucleic acid stability and/or specificity. In addition, miRNA mimics can include overhangs. The overhangs can consist of 1-6 nucleotides on either the 3′ or 5′ end of either strand and can be modified to enhance stability or functionality. In one embodiment, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

Antimir or miRNA Inhibitor

The terms “antimir,” “microRNA inhibitor,” “miR inhibitor,” or “inhibitor,” are synonymous and refer to oligonucleotides or modified oligonucleotides that interfere with the ability of specific miRNAs. In general, the inhibitors are nucleic acid or modified nucleic acids in nature including oligonucleotides comprising RNA, modified RNA, DNA, modified DNA, locked nucleic acids (LNAs), or any combination of the above. Modifications include 2′ modifications (including 2′-0 alkyl modifications and 2′ F modifications) and internucleotide modifications (e.g. phosphorothioate modifications) that can affect delivery, stability, specificity, intracellular compartmentalization, or potency. In addition, miRNA inhibitors can comprise conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors comprise contain one or more sequences or portions of sequences that are complementary or partially complementary with the mature strand (or strands) of the miRNA to be targeted, in addition, the miRNA inhibitor may also comprise additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

### U1 Adaptor

U1 adaptor inhibit polyA sites and are bifunctional oligonucleotides with a target domain complementary to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant Mol Biol 49:77-95). Nucleotides 2-11 of the 5′ end of U1 snRNA base pair bind with the 5′ss of the pre mRNA. In one embodiment, oligonucleotides are U1 adaptors. In one embodiment, the U1 adaptor can be administered in combination with at least one other iRNA agent.

### Cationic Lipids

Cationic lipids can have certain design features including a head group, one or more hydrophobic tails, and a linker between the head group and the one or more tails. The head group can include an amine; for example an amine having a desired pKₐ. The pKₐ can be influenced by the structure of the lipid, particularly the nature of head group; e.g., the presence, absence, and location of functional groups such as anionic functional groups, hydrogen bond donor functional groups, hydrogen bond acceptor groups, hydrophobic groups (e.g., aliphatic groups), hydrophilic groups (e.g., hydroxyl or methoxy), or aryl groups. The head group amine can be a cationic amine; a primary, secondary, or tertiary amine; the head group can include one amine group (monoamine), two amine groups (diamine), three amine groups (triamine), or a larger number of amine groups, as in an oligoamine or polyamine. The head group can include a functional group that is less strongly basic than an amine, such as, for example, an imidazole, a pyridine, or a guanidinium group. The head group can be zwitterionic. Other head groups are suitable as well.

The one or more hydrophobic tails can include two hydrophobic chains, which may be the same or different. The tails can be aliphatic; for example, they can be composed of carbon and hydrogen, either saturated or unsaturated but without aromatic rings. The tails can be fatty acid tails; some such groups include octanyl, nonanyl, decyl, lauryl, myristyl, palmityl, stearyl, α-linoleyl, stearidonyl, linoleyl, γ-linolenyl, arachadonyl, oleyl, and others. Other hydrophobic tails are suitable as well.

The linker can include, for example, a glyceride linker, an acyclic glyceride analog linker, or a cyclic linker (including a spiro linker, a bicyclic linker, and a polycyclic linker). The linker can include functional groups such as an ether, an ester, a phosphate, a phosphonate, a phosphorothioate, a sulfonate, a disulfide, an acetal, a ketal, an imine, a hydrazone, or an oxime. Other linkers and functional groups are suitable as well.

A number of cationic lipids, and methods for making them, are described in, for example, in application nos. WO 2010/054406, WO/2010/054401, WO/2010/054405, and WO/2010/054384, each filed Nov. 10, 2009, and applications referred to therein, including Nos. 61/104,219, filed Oct. 9, 2008; No. 61/113,179, filed Nov. 10, 2008; No. 61/154,350, filed Feb. 20, 2009; No. 61/171,439, filed Apr. 21, 2009; No. 61/175,770, filed May 5, 2009; No. 61/185,438, filed Jun. 9, 2009; No. 61/225,898, filed Jul. 15, 2009; and No. 61/234,098, filed Aug. 14, 2009; WO 2009/086558; and WO 2008/042973. Each of these documents is incorporated by reference in its entirety. See, for example, Tables 1 and 2 of application no. WO/2010/054406, filed Nov. 10, 2009, at pages 33-51.

In particular embodiments, the lipids are cationic lipids. As used herein, the term “cationic lipid” is meant to include those lipids having one or two fatty acid or fatty aliphatic chains and an amino head group (including an alkylamino or dialkylamino group) that may be protonated to form a cationic lipid at physiological pH. In some embodiments, a cationic lipid is referred to as an “amino lipid.”

Other cationic lipids would include those having alternative fatty acid groups and other dialkylamino groups, including those in which the alkyl substituents are different (e.g., N-ethyl-N-methylamino-, N-propyl-N-ethylamino- and the like). For those embodiments in which R₁ and R₂ are both long chain alkyl, alkenyl, alkynyl, or acyl groups, they can be the same or different. In general, lipids (e.g., a cationic lipid) having less-saturated acyl chains are more easily sized, particularly when the complexes are sized below about 0.3 microns, for purposes of filter sterilization. Cationic lipids containing unsaturated fatty acids with carbon chain lengths in the range of C₁₀ to C₂₀ are typical. Other scaffolds can also be used to separate the amino group (e.g., the amino group of the cationic lipid) and the fatty acid or fatty alkyl portion of the cationic lipid. Suitable scaffolds are known to those of skill in the art.

In certain embodiments, cationic lipids have at least one protonatable or deprotonatable group, such that the lipid is positively charged at a pH at or below physiological pH (e.g. pH 7.4), and neutral at a second pH, preferably at or above physiological pH. Such lipids are also referred to as cationic lipids. It will, of course, be understood that the addition or removal of protons as a function of pH is an equilibrium process, and that the reference to a charged or a neutral lipid refers to the nature of the predominant species and does not require that all of the lipid be present in the charged or neutral form. The lipids can have more than one protonatable or deprotonatable group, or can be zwiterrionic.

In certain embodiments, protonatable lipids (i.e., cationic lipids) have a pKₐ of the protonatable group in the range of about 4 to about 11. Typically, lipids will have a pKₐ of about 4 to about 7, e.g., between about 5 and 7, such as between about 5.5 and 6.8, when incorporated into lipid particles. Such lipids will be cationic at a lower pH formulation stage, while particles will be largely (though not completely) surface neutralized at physiological pH around pH 7.4. One of the benefits of a pKₐ in the range of between about 4 and 7 is that at least some nucleic acid associated with the outside surface of the particle will lose its electrostatic interaction at physiological pH and be removed by simple dialysis; thus greatly reducing the particle's susceptibility to clearance. pKₐ measurements of lipids within lipid particles can be performed, for example, by using the fluorescent probe 2-(p-toluidino)-6-napthalene sulfonic acid (TNS), using methods described in Cullis et al., (1986) Chem Phys Lipids 40, 127-144, which is incorporated by reference in its entirety.

In particular embodiments, the lipids are charged lipids. As used herein, the term “charged lipid” is meant to include those lipids having one or two fatty acyl or fatty alkyl chains and a quaternary amino head group. The quaternary amine carries a permanent positive charge. The head group can optionally include a ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine) In some embodiments, a charged lipid is referred to as an “amino lipid.” See, for example, provisional U.S. patent application 61/267,419, filed Dec. 7, 2009, which is incorporated by reference in its entirety.

In particular embodiments, lipid particles include one or more cationic lipids selected from DLin-K-DMA, DLinDMA, DLinDAP, DLin-K-C2-DMA, and DLin-K²-DMA. The structures of DLin-K-DMA, DLinDMA, DLinDAP, DLin-K-C2-DMA, and DLin-K²-DMA are provided in the Examples. These lipids may be synthesized as described in these Examples. In particular embodiments, the cationic lipid component of the lipid particle consists of DLin-K-DMA, DLinDMA, DLinDAP, DLin-K-C2-DMA, or DLin-K²-DMA.

In other embodiments, lipid particles include one or more additional cationic lipids. Other cationic lipids that may be used in the lipid particles of the present invention include, but are not limited to, N,N-dioleyl-N,N-dimethylammonium chloride (DODAC), dioctadecyldimethylammonium (DODMA), distearyldimethylammonium (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3β-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), and mixtures thereof. A number of these lipids and related analogs have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, each of which is incorporated by reference in its entirety. Other cationic lipids that may be used include those described in International Patent Application No. PCT/US2008/088676 and/or U.S. Provisional Patent Application No. 61/104,212, each of which is incorporated by reference in its entirety.

The cationic lipid typically comprises from about 50 mol % to about 85 mol %, about 50 mol % to about 80 mol %, about 50 mol % to about 75 mol %, about 50 mol % to about 65 mol %, or about 55 mol % to about 65 mol % of the total lipid present in the particle. It will be readily apparent to one of skill in the art that depending on the intended use of the particles, the proportions of the components can be varied and the delivery efficiency of a particular formulation can be measured using, e.g., an endosomal release parameter (ERP) assay.

### Apolipoproteins

The formulations can further comprise an apolipoprotein. As used herein, the term “apolipoprotein” or “lipoprotein” refers to apolipoproteins known to those of skill in the art and variants and fragments thereof and to apolipoprotein agonists, analogues or fragments thereof described below.

Suitable apolipoproteins include, but are not limited to, ApoA-I, ApoA-II, ApoA-IV, ApoA-V and ApoE, and active polymorphic forms, isoforms, variants and mutants as well as fragments or truncated forms thereof. In certain embodiments, the apolipoprotein is a thiol containing apolipoprotein. “Thiol containing apolipoprotein” refers to an apolipoprotein, variant, fragment or isoform that contains at least one cysteine residue. The most common thiol containing apolipoproteins are ApoA-I Milano (ApoA-I_{M}) and ApoA-I Paris (ApoA-I_{P}) which contain one cysteine residue (Jia et al., 2002, Biochem. Biophys. Res. Comm. 297: 206-13; Bielicki and Oda, 2002, Biochemistry 41: 2089-96). ApoA-II, ApoE2 and ApoE3 are also thiol containing apolipoproteins. Isolated ApoE and/or active fragments and polypeptide analogues thereof, including recombinantly produced forms thereof, are described in U.S. Pat. Nos. 5,672,685; 5,525,472; 5,473,039; 5,182,364; 5,177,189; 5,168,045; 5,116,739; the disclosures of which are herein incorporated by reference. ApoE3 is disclosed in Weisgraber, et al., “Human E apoprotein heterogeneity: cysteine-arginine interchanges in the amino acid sequence of the apo-E isoforms,” J. Biol. Chem. (1981) 256: 9077-9083; and Rall, et al., “Structural basis for receptor binding heterogeneity of apolipoprotein E from type III hyperlipoproteinemic subjects,” Proc. Nat. Acad. Sci. (1982) 79: 4696-4700. See also GenBank accession number K00396.

In certain embodiments, the apolipoprotein can be in its mature form, in its preproapolipoprotein form or in its proapolipoprotein form. Homo- and heterodimers (where feasible) of pro- and mature ApoA-I (Duverger et al., 1996, Arterioscler. Thromb. Vasc. Biol. 16(12):1424-29), ApoA-I Milano (Klon et al., 2000, Biophys. J. 79:(3)1679-87; Franceschini et al., 1985, J. Biol. Chem. 260: 1632-35), ApoA-I Paris (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), and ApoE (McLean et al., 1983, J. Biol. Chem. 258(14):8993-9000) can also be utilized.

In certain embodiments, the apolipoprotein can be a fragment, variant or isoform of the apolipoprotein. The term “fragment” refers to any apolipoprotein having an amino acid sequence shorter than that of a native apolipoprotein and which fragment retains the activity of native apolipoprotein, including lipid binding properties. By “variant” is meant substitutions or alterations in the amino acid sequences of the apolipoprotein, which substitutions or alterations, e.g., additions and deletions of amino acid residues, do not abolish the activity of native apolipoprotein, including lipid binding properties. Thus, a variant can comprise a protein or peptide having a substantially identical amino acid sequence to a native apolipoprotein provided herein in which one or more amino acid residues have been conservatively substituted with chemically similar amino acids. Examples of conservative substitutions include the substitution of at least one hydrophobic residue such as isoleucine, valine, leucine or methionine for another. Likewise, for example, the substitution of at least one hydrophilic residue such as, for example, between arginine and lysine, between glutamine and asparagine, and between glycine and serine (see U.S. Pat. Nos. 6,004,925, 6,037,323 and 6,046,166) are conservative substitutions. The term “isoform” refers to a protein having the same, greater or partial function and similar, identical or partial sequence, and may or may not be the product of the same gene and usually tissue specific (see Weisgraber 1990, J. Lipid Res. 31(8):1503-11; Hixson and Powers 1991, J. Lipid Res. 32(9):1529-35; Lackner et al., 1985, J. Biol. Chem. 260(2):703-6; Hoeg et al., 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vase. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sacre et al., 2003, FEBS Lett. 540(1-3):181-7; Weers, et al., 2003, Biophys. Chem. 100(1-3):481-92; Gong et al., 2002, J. Biol. Chem. 277(33):29919-26; Ohta et al., 1984, J. Biol. Chem. 259(23):14888-93 and U.S. Pat. No. 6,372,886).

In certain embodiments, the methods and compositions include the use of a chimeric construction of an apolipoprotein. For example, a chimeric construction of an apolipoprotein can be comprised of an apolipoprotein domain with high lipid binding capacity associated with an apolipoprotein domain containing ischemia reperfusion protective properties. A chimeric construction of an apolipoprotein can be a construction that includes separate regions within an apolipoprotein (i.e., homologous construction) or a chimeric construction can be a construction that includes separate regions between different apolipoproteins (i.e., heterologous constructions). Compositions comprising a chimeric construction can also include segments that are apolipoprotein variants or segments designed to have a specific character (e.g., lipid binding, receptor binding, enzymatic, enzyme activating, antioxidant or reduction-oxidation property) (see Weisgraber 1990, J. Lipid Res. 31(8):1503-11; Hixson and Powers 1991, J. Lipid Res. 32(9):1529-35; Lackner et al., 1985, J. Biol. Chem. 260(2):703-6; Hoeg et al, 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vasc. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sorenson et al., 1999, Arterioscler. Thromb. Vasc. Biol. 19(9):2214-25; Palgunachari 1996, Arterioscler. Throb. Vasc. Biol. 16(2):328-38: Thurberg et al., J. Biol. Chem. 271(11):6062-70; Dyer 1991, J. Biol. Chem. 266(23):150009-15; Hill 1998, J. Biol. Chem. 273(47):30979-84).

Apolipoproteins utilized also include recombinant, synthetic, semi-synthetic or purified apolipoproteins. Methods for obtaining apolipoproteins or equivalents thereof are well-known in the art. For example, apolipoproteins can be separated from plasma or natural products by, for example, density gradient centrifugation or immunoaffinity chromatography, or produced synthetically, semi-synthetically or using recombinant DNA techniques known to those of the art (see, e.g., Mulugeta et al., 1998, J. Chromatogr. 798(1-2): 83-90; Chung et al., 1980, J. Lipid Res. 21(3):284-91; Cheung et al., 1987, J. Lipid Res. 28(8):913-29; Persson, et al., 1998, J. Chromatogr. 711:97-109; U.S. Pat. Nos. 5,059,528, 5,834,596, 5,876,968 and 5,721,114; and PCT Publications WO 86/04920 and WO 87/02062).

Apolipoproteins further include apolipoprotein agonists such as peptides and peptide analogues that mimic the activity of ApoA-I, ApoA-I Milano (ApoA-I_{M}), ApoA-I Paris (ApoA-I_{P}), ApoA-II, ApoA-IV, and ApoE. For example, the apolipoprotein can be any of those described in U.S. Pat. Nos. 6,004,925, 6,037,323, 6,046,166, and 5,840,688, the contents of which are incorporated herein by reference in their entireties.

Apolipoprotein agonist peptides or peptide analogues can be synthesized or manufactured using any technique for peptide synthesis known in the art including, e.g., the techniques described in U.S. Pat. Nos. 6,004,925, 6,037,323 and 6,046,166. For example, the peptides may be prepared using the solid-phase synthetic technique initially described by Merrifield (1963, J. Am. Chem. Soc. 85:2149-2154). Other peptide synthesis techniques may be found in Bodanszky et al., Peptide Synthesis, John Wiley & Sons, 2d Ed., (1976) and other references readily available to those skilled in the art. A summary of polypeptide synthesis techniques can be found in Stuart and Young, Solid Phase Peptide. Synthesis, Pierce Chemical Company, Rockford, Ill., (1984). Peptides may also be synthesized by solution methods as described in The Proteins, Vol. II, 3d Ed., Neurath et. al., Eds., p. 105-237, Academic Press, New York, N.Y. (1976). Appropriate protective groups for use in different peptide syntheses are described in the above-mentioned texts as well as in McOmie, Protective Groups in Organic Chemistry, Plenum Press, New York, N.Y. (1973). The peptides might also be prepared by chemical or enzymatic cleavage from larger portions of, for example, apolipoprotein A-I.

In certain embodiments, the apolipoprotein can be a mixture of apolipoproteins. In one embodiment, the apolipoprotein can be a homogeneous mixture, that is, a single type of apolipoprotein. In another embodiment, the apolipoprotein can be a heterogeneous mixture of apolipoproteins, that is, a mixture of two or more different apolipoproteins. Embodiments of heterogenous mixtures of apolipoproteins can comprise, for example, a mixture of an apolipoprotein from an animal source and an apolipoprotein from a semi-synthetic source. In certain embodiments, a heterogenous mixture can comprise, for example, a mixture of ApoA-I and ApoA-I Milano. In certain embodiments, a heterogeneous mixture can comprise, for example, a mixture of ApoA-I Milano and ApoA-I Paris. Suitable mixtures for use in the methods and compositions described herein will be apparent to one of skill in the art.

If the apolipoprotein is obtained from natural sources, it can be obtained from a plant or animal source. If the apolipoprotein is obtained from an animal source, the apolipoprotein can be from any species. In certain embodiments, the apolipoprotein can be obtained from an animal source. In certain embodiments, the apolipoprotein can be obtained from a human source. In preferred embodiments, the apolipoprotein is derived from the same species as the individual to which the apolipoprotein is administered.

### Lipid Particles

Lipid particles can include one or more of the cationic lipids described above. Lipid particles include, but are not limited to, liposomes. As used herein, a liposome is a structure having lipid-containing membranes enclosing an aqueous interior. Liposomes may have one or more lipid membranes. Liposomes can be single-layered, referred to as unilamellar, or multi-layered, referred to as multilamellar. When complexed with nucleic acids, lipid particles may also be lipoplexes, which are composed of cationic lipid bilayers sandwiched between DNA layers, as described, e.g., in Felgner, Scientific American.

The lipid particles may further comprise one or more additional lipids and/or other components such as cholesterol. Other lipids may be included in the liposome compositions for a variety of purposes, such as to prevent lipid oxidation or to attach ligands onto the liposome surface. Any of a number of lipids may be present in liposomes, including amphipathic, neutral, cationic, and anionic lipids. Such lipids can be used alone or in combination. Specific examples of additional lipid components that may be present are described below.

Additional components that may be present in a lipid particle include bilayer stabilizing components such as polyamide oligomers (see, e.g., U.S. Pat. No. 6,320,017, which is incorporated by reference in its entirety), peptides, proteins, detergents, lipid-derivatives, such as PEG coupled to phosphatidylethanolamine and PEG conjugated to ceramides (see, U.S. Pat. No. 5,885,613, which is incorporated by reference in its entirety).

In particular embodiments, the lipid particles include one or more of a second amino lipid or cationic lipid, a neutral lipid, a sterol, and a lipid selected to reduce aggregation of lipid particles during formation, which may result from steric stabilization of particles which prevents charge-induced aggregation during formation.

Lipid particles can include two or more cationic lipids. The lipids can be selected to contribute different advantageous properties. For example, cationic lipids that differ in properties such as amine pKₐ, chemical stability, half-life in circulation, half-life in tissue, net accumulation in tissue, or toxicity can be used in a lipid particle. In particular, the cationic lipids can be chosen so that the properties of the mixed-lipid particle are more desirable than the properties of a single-lipid particle of individual lipids.

Net tissue accumulation and long term toxicity (if any) from the cationic lipids can be modulated in a favorable way by choosing mixtures of cationic lipids instead of selecting a single cationic lipid in a given formulation. Such mixtures can also provide better encapsulation and/or release of the drug. A combination of cationic lipids also can affect the systemic stability when compared to single entity in a formulation.

In one example, a series of structurally similar compounds can have varying pKₐ values that span a range, e.g. of less than 1 pKₐ unit, from 1 to 2 pKₐ units, or a range of more than 2 pKₐ units. Within the series, it may be found that a pKₐ in the middle of the range is associated with an enhancement of advantageous properties (greater effectiveness) or a decrease in disadvantageous properties (e.g., reduced toxicity), compared to compounds having pKₐ values toward the ends of the range. In such a case, two (or more) different compounds having pKₐ values toward opposing ends of the range can be selected for use together in a lipid particle. In this way, the net properties of the lipid particle (for instance, charge as a function of local pH) can be closer to that of a particle including a single lipid from the middle of the range. Cationic lipids that are structurally dissimilar (for example, not part of the series of structurally similar compounds mentioned above) can also be used in a mixed-lipid particle.

In some cases, two or more different cationic lipids may have widely differing pKₐ values, e.g., differing by 3 or more pKₐ units. In this case, the net behavior of a mixed lipid particle will not necessarily mimic that of a single-lipid particle having an intermediate pKₐ. Rather, the net behavior may be that of a particle having two distinct protonatable (or deprotonatable, as the case may be) site with different pKₐ values. In the case of a single lipid, the fraction of protonatable sites that are in fact protonated varies sharply as the pH moves from below the pKₐ to above the pKₐ (when the pH is equal to the pKₐ value, 50% of the sites are protonated). When two or more different cationic lipids may have widely differing pKₐ values (e.g., differing by 3 or more pKₐ units) are combined in a lipid particle, the lipid particle can show a more gradual transition from non-protonated to protonated as the pH is varied.

In other examples, two or more lipids may be selected based on other considerations. For example, if one lipid by itself is highly effective but moderately toxic, it might be combined with a lipid that is less effective but non-toxic. In some cases, the combination can remain highly effective but have a greatly reduced toxicity, even where it might be predicted that the combination would be only moderately effective and only slightly less toxic.

The selection may be guided by a measured value of an experimentally determinable characteristic, e.g., a characteristic that can be assigned a numerical value from the results of an experiment. Experimentally determinable characteristics can include a measure of safety, a measure of efficacy, a measure of interaction with a predetermined biomolecule, or pKₐ.

A measure of safety might include a survival rate, an LD₅₀, or a level of a biomarker (such as a serum biomarker) associated with tissue damage (e.g., liver enzymes for liver; CPK for muscle; ionic balance for kidney). A measure of efficacy can be any measurement that indicates whether a therapeutic agent is producing an effect; particularly, whether and/or to what degree it is producing a desired effect, such as treating, preventing, ameliorating, or otherwise improving a disease, disorder, or other clinical condition. The measure of efficacy can be an indirect measure; for example, if a therapeutic agent is intended to produce a particular effect at a cellular level, measurements of that effect on cell cultures can be a measure of efficacy. A measure of interaction with predetermined biomolecules can include a K_{d} for binding to a particular protein or a measure of the character, degree or extent of interaction with other lipids, including cellular substructures such as cell membranes, endosomal membranes, nuclear membranes, and the like.

The cationic lipids can be selected on the basis of mechanism of action, e.g., whether, under what conditions, or to what extent the lipids interact with predetermined biomolecules. For example, a first cationic lipid can be chosen, in part, because it is associated with an ApoE-dependent mechanism; a second cationic lipid can be chosen, in part, because it is associated with an ApoE-independent mechanism.

For example, a lipid particle can contain a mixture of the cationic lipids described in, e.g., WO 2009/086558, and provisional U.S. Application No. 61/104,219, filed Oct. 9, 2008 (each of which is incorporated by reference in its entirety), and ester analogs thereof. In another example, a lipid particle can contain a mixture of a lipid, for example, Lipid A, described in PCT/US10/22614, filed Jan. 29, 2010 and a lipid, for example, the lipid of formula V or formula VI, described in U.S. Provisional Application 61/175,770, filed May 5, 2009.

Examples of lipids that reduce aggregation of particles during formation include polyethylene glycol (PEG)-modified lipids, monosialoganglioside Gm1, and polyamide oligomers (“PAO”) such as (described in U.S. Pat. No. 6,320,017, which is incorporated by reference in its entirety). Other compounds with uncharged, hydrophilic, steric-barrier moieties, which prevent aggregation during formulation, like PEG, Gm1 or ATTA, can also be coupled to lipids. ATTA-lipids are described, e.g., in U.S. Pat. No. 6,320,017, and PEG-lipid conjugates are described, e.g., in U.S. Pat. Nos. 5,820,873, 5,534,499 and 5,885,613, each of which is incorporated by reference in its entirety. Typically, the concentration of the lipid component selected to reduce aggregation is about 1 to 15% (by mole percent of lipids).

Specific examples of PEG-modified lipids (or lipid-polyoxyethylene conjugates) that can have a variety of “anchoring” lipid portions to secure the PEG portion to the surface of the lipid vesicle include PEG-modified phosphatidylethanolamine and phosphatidic acid, PEG-ceramide conjugates (e.g., PEG-CerC14 or PEG-CerC20) which are described in U.S. Pat. No. 5,820,873, incorporated herein by reference, PEG-modified dialkylamines and PEG-modified 1,2-diacyloxypropan-3-amines. Particularly preferred are PEG-modified diacylglycerols and dialkylglycerols.

In embodiments where a sterically-large moiety such as PEG or ATTA are conjugated to a lipid anchor, the selection of the lipid anchor depends on what type of association the conjugate is to have with the lipid particle. It is well known that mPEG (mw2000)-diastearoylphosphatidylethanolamine (PEG-DSPE) will remain associated with a liposome until the particle is cleared from the circulation, possibly a matter of days. Other conjugates, such as PEG-CerC20 have similar staying capacity. PEG-CerC14, however, rapidly exchanges out of the formulation upon exposure to serum, with a T_{1/2} less than 60 min in some assays. As illustrated in U.S. Pat. No. 5,820,873, at least three characteristics influence the rate of exchange: length of acyl chain, saturation of acyl chain, and size of the steric-barrier head group. Compounds having suitable variations of these features may be useful. For some therapeutic applications it may be preferable for the PEG-modified lipid to be rapidly lost from the nucleic acid-lipid particle in vivo and hence the PEG-modified lipid will possess relatively short lipid anchors. In other therapeutic applications it may be preferable for the nucleic acid-lipid particle to exhibit a longer plasma circulation lifetime and hence the PEG-modified lipid will possess relatively longer lipid anchors.

It should be noted that aggregation preventing compounds do not necessarily require lipid conjugation to function properly. Free PEG or free ATTA in solution may be sufficient to prevent aggregation. If the particles are stable after formulation, the PEG or ATTA can be dialyzed away before administration to a subject.

Neutral lipids, when present in the lipid particle, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C₁₀ to C₂₀ are preferred. In another group of embodiments, lipids with mono or diunsaturated fatty acids with carbon chain lengths in the range of C₁₀ to C₂₀ are used. Additionally, lipids having mixtures of saturated and unsaturated fatty acid chains can be used. Preferably, the neutral lipids used are DOPE, DSPC, POPC, DPPC or any related phosphatidylcholine. The neutral lipids may also be composed of sphingomyelin, dihydrosphingomyeline, or phospholipids with other head groups, such as serine and inositol.

The sterol component of the lipid mixture, when present, can be any of those sterols conventionally used in the field of liposome, lipid vesicle or lipid particle preparation. A preferred sterol is cholesterol.

Other cationic lipids, which carry a net positive charge at about physiological pH, in addition to those specifically described above, may also be included in lipid particles. Such cationic lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). In particular embodiments, a cationic lipid is an amino lipid.

Anionic lipids suitable for use in lipid particles include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In numerous embodiments, amphipathic lipids are included in lipid particles. “Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Also suitable for inclusion in the lipid particles are programmable fusion lipids or fusion-promoting lipid. Such lipid particles have little tendency to fuse with cell membranes and deliver their payload until a given signal event occurs. This allows the lipid particle to distribute more evenly after injection into an organism or disease site before it starts fusing with cells. The signal event can be, for example, a change in pH, temperature, ionic environment, or time. The fusion promoting-lipids can be, for example, compounds of formula (I) as described above. In some cases, the signal event can be a change in pH, for example, such as the difference in pH between an extracelluar environment and an intracellular environment, or between an intracellular environment and an endosomal environment.

When time is the signal event, a fusion delaying or “cloaking” component, such as an ATTA-lipid conjugate or a PEG-lipid conjugate, can simply exchange out of the lipid particle membrane over time. By the time the lipid particle is suitably distributed in the body, it has lost sufficient cloaking agent so as to be fusogenic. With other signal events, it can be desirable to choose a signal that is associated with the disease site or target cell, such as increased temperature at a site of inflammation.

In certain embodiments, it is desirable to target the lipid particles using targeting moieties that are specific to a cell type or tissue. Targeting of lipid particles using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044, each of which is incorporated by reference in its entirety). The targeting moieties can comprise the entire protein or fragments thereof. Targeting mechanisms generally require that the targeting agents be positioned on the surface of the lipid particle in such a manner that the target moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002).

The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)).

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

In some embodiments, the lipid particle includes a mixture of a cationic lipid and a fusion-promoting lipid. The lipid particle can further include a neutral lipid, a sterol, a PEG-modified lipid, or a combination of these. For example, the lipid particle can include a cationic lipid, a fusion-promoting lipid, and a neutral lipid, but no sterol or PEG-modified lipid. The lipid particle can include a cationic lipid, a fusion-promoting lipid, and a neutral lipid, but no sterol or PEG-modified lipid. The lipid particle can include a cationic lipid, a fusion-promoting lipid, and a PEG-modified lipid, but no sterol or neutral lipid. The lipid particle can include a cationic lipid, a fusion-promoting lipid, a sterol, and a neutral lipid, but no PEG-modified lipid. The lipid particle can include a cationic lipid, a fusion-promoting lipid, a sterol, and a PEG-modified lipid, but no neutral lipid. The lipid particle can include a cationic lipid, a fusion-promoting lipid, a neutral lipid, and a PEG-modified lipid, but no sterol. The lipid particle can include a cationic lipid, a fusion-promoting lipid, a sterol, neutral lipid, and a PEG-modified lipid.

In one exemplary embodiment, the lipid particle comprises a mixture of a cationic lipid, a fusion-promoting lipid, neutral lipids (other than a cationic lipid), a sterol (e.g., cholesterol) and a PEG-modified lipid (e.g., a PEG-DMG or PEG-DMA). In certain embodiments, the lipid mixture consists of or consists essentially of a cationic lipid, a fusion-promoting lipid, a neutral lipid, cholesterol, and a PEG-modified lipid. In further preferred embodiments, the lipid particle includes the above lipid mixture in molar ratios of about 20-70% cationic lipid: 0.1-50% fusion promoting lipid: 5-45% neutral lipid: 20-55% cholesterol: 0.5-15% PEG-modified lipid. In some embodiments, the fusion-promoting lipid can be present in a molar ratio of 0.1-50%, 0.5-50%, 1-50%, 5%-45%, 10%-40%, or 15%-35%. In some embodiments, the fusion-promoting lipid can be present in a molar ratio of 0.1-50%, 0.5-50%, 1-50%, 5%-45%, 10%-40%, or 15%-35%. In some embodiments, the fusion-promoting lipid can be present in a molar ratio of 0.1-50%, 10-50%, 20-50%, or 30-50%. In some embodiments, the fusion-promoting lipid can be present in a molar ratio of 0.1-50%, 0.5-45%, 1-40%, 1%-35%, 1%-30%, or 1%-20%.

In further preferred embodiments, the lipid particle consists of or consists essentially of the above lipid mixture in molar ratios of about 20-70% cationic lipid: 0.1-50% fusion promoting lipid: 5-45% neutral lipid: 20-55% cholesterol: 0.5-15% PEG-modified lipid.

In particular embodiments, the lipid particle comprises, consists of, or consists essentially of a mixture of cationic lipids chosen from, for example, those described in application nos. PCT/US09/63933, PCT/US09/63927, PCT/US09/63931, and PCT/US09/63897, each filed Nov. 10, 2009, and applications referred to therein, including Nos. 61/104,219, filed Oct. 9, 2008; No. 61/113,179, filed Nov. 10, 2008; No. 61/154,350, filed Feb. 20, 2009; No. 61/171,439, filed Apr. 21, 2009; No. 61/175,770, filed May 5, 2009; No. 61/185,438, filed Jun. 9, 2009; No. 61/225,898, filed Jul. 15, 2009; No. 61/234,098, filed Aug. 14, 2009; and 61/287,995, filed Dec. 18, 2009; WO 2009/086558; and WO 2008/042973 (each of these documents is incorporated by reference in its entirety. See, for example, Tables 1 and 2 of application no. PCT/US09/63933, filed Nov. 10, 2009, at pages 33-51, and Tables 1-4 and 9 of 61/287,995, at pages 28-53 and 135-141), DSPC, Chol, and either PEG-DMG or PEG-DMA, e.g., in a molar ratio of about 20-60% cationic lipid: 0.1-50% fusion-promoting lipid: 5-25% DSPC:25-55% Chol:0.5-15% PEG-DMG or PEG-DMA. In particular embodiments, the molar lipid ratio, with regard to mol % cationic lipid/DSPC/Chol/PEG-DMG or PEG-DMA) is approximately 40/10/40/10, 35/15/40/10 or 52/13/30/5; this mixture is further combined with a fusion-promoting lipid in a molar ratio of 0.1-50%, 0.1-50%, 0.5-50%, 1-50%, 5%-45%, 10%-40%, or 15%-35%; in other words, when a 40/10/40/10 mixture of lipid/DSPC/Chol/PEG-DMG or PEG-DMA is combined with a fusion-promoting peptide in a molar ratio of 50%, the resulting lipid particles can have a total molar ratio of (mol % cationic lipid/DSPC/Chol/PEG-DMG or PEG-DMA/fusion-promoting peptide) 20/5/20/5/50. In another group of embodiments, the neutral lipid, DSPC, in these compositions is replaced with POPC, DPPC, DOPE or SM.

### Methods of Producing Lipid Particles

Lipid particles of the present invention may be prepared by procedures described in the art, including those described in WO 96/40964, WO 01/05374, U.S. Pat. No. 5,981,501, U.S. Pat. No. 6,110,745, WO 1999/18933, and WO 1998/51278. In the exemplary methods described herein, a mixture of lipids is combined with a buffered aqueous solution of nucleic acid to produce an intermediate mixture containing nucleic acid encapsulated in lipid particles, e.g., wherein the encapsulated nucleic acids are present in a nucleic acid/lipid ratio of about 10 wt % to about 20 wt %. The intermediate mixture may optionally be sized to obtain lipid-encapsulated nucleic acid particles wherein the lipid portions are unilamellar vesicles, preferably having a diameter of 30 to 150 nm, more preferably about 40 to 90 nm. The pH may then be raised to neutralize at least a portion of the surface charges on the lipid particles, thus providing an at least partially surface-neutralized lipid particle composition.

In preparing the lipid particles of the invention, the mixture of lipids is typically a solution of lipids in an organic solvent. This mixture of lipids can then be dried to form a thin film or lyophilized to form a powder before being hydrated with an aqueous buffer to form liposomes. Alternatively, in a preferred method, the lipid mixture can be solubilized in a water miscible alcohol, such as ethanol, and this ethanolic solution added to an aqueous buffer resulting in spontaneous liposome formation. In most embodiments, the alcohol is used in the form in which it is commercially available. For example, ethanol can be used as absolute ethanol (100%), or as 95% ethanol, the remainder being water. This method is described in more detail in U.S. Pat. No. 5,976,567.

In one exemplary embodiment, the mixture of lipids is a mixture of cationic lipids, non-cationic lipids, a sterol (e.g., cholesterol) and a PEG-modified lipid in an alcohol solvent. In preferred embodiments, the lipid mixture consists essentially of a cationic lipid, a non-cationic lipid, cholesterol and a PEG-modified lipid in alcohol, more preferably ethanol. In further preferred embodiments, the first solution consists of the above lipid mixture in molar ratios of about 20-70% cationic lipid: 5-45% non-cationic lipid:20-55% cholesterol:0.5-15% PEG-modified lipid.

In accordance with the invention, the lipid mixture is combined with a buffered aqueous solution that may contain the nucleic acids. The buffered aqueous solution is typically a solution in which the buffer has a pH of less than the pKₐ of the protonatable lipid in the lipid mixture. Examples of suitable buffers include citrate, phosphate, acetate, and MES. A particularly preferred buffer is citrate buffer. Preferred buffers will be in the range of 1-1000 mM of the anion, depending on the chemistry of the nucleic acid being encapsulated, and optimization of buffer concentration may be significant to achieving high loading levels (see, e.g., U.S. Pat. No. 6,287,591 and U.S. Pat. No. 6,858,225). Alternatively, pure water acidified to pH 5-6 with chloride, sulfate or the like may be useful. In this case, it may be suitable to add 5% glucose, or another non-ionic solute which will balance the osmotic potential across the particle membrane when the particles are dialyzed to remove ethanol, increase the pH, or mixed with a pharmaceutically acceptable carrier such as normal saline. The amount of nucleic acid in buffer can vary, but will typically be from about 0.01 mg/mL to about 200 mg/mL, more preferably from about 0.5 mg/mL to about 50 mg/mL.

The mixture of lipids and the buffered aqueous solution of nucleic acids is combined to provide an intermediate mixture. The intermediate mixture is typically a mixture of lipid particles having encapsulated nucleic acids. Additionally, the intermediate mixture may also contain some portion of nucleic acids which are attached to the surface of the lipid particles (liposomes or lipid vesicles) due to the ionic attraction of the negatively-charged nucleic acids and positively-charged lipids on the lipid particle surface (the cationic lipids or other lipid making up the protonatable first lipid component are positively charged in a buffer having a pH of less than the pKₐ of the protonatable group on the lipid). In one group of preferred embodiments, the mixture of lipids is an alcohol solution of lipids and the volumes of each of the solutions is adjusted so that upon combination, the resulting alcohol content is from about 20% by volume to about 45% by volume. The method of combining the mixtures can include any of a variety of processes, often depending upon the scale of formulation produced. For example, when the total volume is about 10-20 mL or less, the solutions can be combined in a test tube and stirred together using a vortex mixer. Large-scale processes can be carried out in suitable production scale glassware.

Optionally, the lipid particles that are produced by combining the lipid mixture and the buffered aqueous solution of nucleic acids can be sized to achieve a desired size range and relatively narrow distribution of lipid particle sizes. Preferably, the compositions provided herein will be sized to a mean diameter of from about 70 to about 200 nm, more preferably about 90 to about 130 nm. Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles (SUVs) less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. In both methods, the particle size distribution can be monitored by conventional laser-beam particle size determination. For certain methods herein, extrusion is used to obtain a uniform vesicle size.

Extrusion of lipid particles through a small-pore polycarbonate membrane or an asymmetric ceramic membrane results in a relatively well-defined size distribution. Typically, the suspension is cycled through the membrane one or more times until the desired liposome complex size distribution is achieved. The liposomes may be extruded through successively smaller-pore membranes, to achieve a gradual reduction in lipid particle size. In some instances, the lipid particles which are formed can be used without any sizing.

In particular embodiments, methods of the present invention further comprise a step of neutralizing at least some of the surface charges on the lipid portions of the lipid-nucleic acid compositions. By at least partially neutralizing the surface charges, unencapsulated nucleic acid is freed from the lipid particle surface and can be removed from the composition using conventional techniques. Preferably, unencapsulated and surface adsorbed nucleic acids are removed from the resulting compositions through exchange of buffer solutions. For example, replacement of a citrate buffer (pH about 4.0, used for forming the compositions) with a HEPES-buffered saline (HBS pH about 7.5) solution, results in the neutralization of liposome surface and nucleic acid release from the surface. The released nucleic acid can then be removed via chromatography using standard methods, and then switched into a buffer with a pH above the pKa of the lipid used.

Optionally, the lipid vesicles (i.e., lipid particles) can be formed by hydration in an aqueous buffer and sized using any of the methods described above prior to addition of the nucleic acid. As described above, the aqueous buffer should be of a pH below the pKa of the amino lipid. A solution of the nucleic acids can then be added to these sized, preformed vesicles. To allow encapsulation of nucleic acids into such “pre-formed” vesicles the mixture should contain an alcohol, such as ethanol. In the case of ethanol, it should be present at a concentration of about 20% (w/w) to about 45% (w/w). In addition, it may be necessary to warm the mixture of pre-formed vesicles and nucleic acid in the aqueous buffer-ethanol mixture to a temperature of about 25° C. to about 50° C. depending on the composition of the lipid vesicles and the nature of the nucleic acid. It will be apparent to one of ordinary skill in the art that optimization of the encapsulation process to achieve a desired level of therapeutic agent, e.g., nucleic acid, in the lipid vesicles will require manipulation of variable such as ethanol concentration and temperature. Once the therapeutic agents, e.g., nucleic acids, are encapsulated within the preformed vesicles, the external pH can be increased to at least partially neutralize the surface charge. Unencapsulated and surface adsorbed therapeutic agent, e.g., nucleic acids, can then be removed as described above.

### Pharmaceutical Compositions

The lipid particles of present invention may be formulated as a pharmaceutical composition, e.g., which further comprises a pharmaceutically acceptable diluent, excipient, or carrier, such as physiological saline or phosphate buffer, selected in accordance with the route of administration and standard pharmaceutical practice. In particular embodiments, a pharmaceutical composition comprises both a lipid particle and one or more compounds that bind a Na+/K+-ATPase, such as a cardiac glycoside. In a related embodiment, a kit can include both a lipid particle and one or more compounds that bind a Na+/K+-ATPase, such as a cardiac glycoside. The lipid particle and the one or more compounds that both a lipid particle and the one or more compounds that bind a Na+/K+-ATPase, such as a cardiac glycoside, may be present in the same container or in separate containers.

In particular embodiments, pharmaceutical compositions comprising the lipid particles are prepared according to standard techniques and further comprise a pharmaceutically acceptable carrier. Generally, normal saline will be employed as the pharmaceutically acceptable carrier. Other suitable carriers include, e.g., water, buffered water, 0.9% saline, 0.3% glycine, and the like, including glycoproteins for enhanced stability, such as albumin, lipoprotein, globulin, etc. In compositions comprising saline or other salt containing carriers, the carrier is preferably added following nucleic acid lipid particle formation. Thus, after the nucleic acid lipid particle formulations are formed, the compositions can be diluted into pharmaceutically acceptable carriers, such as normal saline.

The resulting pharmaceutical preparations may be sterilized by conventional, well known sterilization techniques. The aqueous solutions can then be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the lipidic suspension may include lipid-protective agents which protect lipids against free-radical and lipid-peroxidative damages on storage. Lipophilic free-radical quenchers, such as α-tocopherol and water-soluble iron-specific chelators, such as ferrioxamine, are suitable.

The concentration of lipid particle in the pharmaceutical formulations can vary widely, i.e., from less than about 0.01%, usually at or at least about 0.05-5% to as much as 10 to 30% by weight and will be selected primarily by fluid volumes, viscosities, etc., in accordance with the particular mode of administration selected.

### EXAMPLES

### Example 1

### Identification of Compounds that Enhance Cellular Uptake and Cytoplasmic Delivery of Lipid Particles

The present example describes the identification of molecules that either enhance uptake or cytoplasmic delivery in the macrophage cell line Raw264.7 using a high throughput screen of known small molecules drugs. In addition, it is shown that for chloroquine, a member of the class of compounds identified as enhancing cytoplasmic release, that a chloroquine lipid (CQ-lipid) can be synthesized where the chloroquine is attached as the lipid headgroup. When the CQ lipid was incorporated into Cy3-labelled siRNA-LN systems, enhanced intracellular delivery was observed. This result was similar to what was observed when cells were treated with free chloroquine, suggesting that the CQ-lipid maintained chloroquine function.

Using LN containing fluorescently labeled siRNA (LN siRNACy3), a library of known small molecule drugs was screened in a 96 well format using the Cellomics Arrrayscan high content screening instrument. Using this high throughput assay in the macrophage cell line Raw264.7, two classes of small molecules that enhance intracellular uptake and cytoplasmic delivery were identified. Drugs such as diprophylline and isoxicam enhanced overall uptake of LN siRNACy3 whereas drugs such as chloroquine increased cytosolic distribution of siRNA. Synthesis of a novel lipid containing a chloroquine motif in the headgroup and its incorporation into the LN delivery system enhanced cytosolic delivery of siRNACy3 concomitant with improved siRNA-mediated gene silencing.

### Materials and Methods

Formulation of Liposomal Nanoparticles

All lipid stocks (DSPC, PEG-s-DMG, Cholesterol, SP-DiOC₁₈, chloroquine lipid and DLinDMA) were dissolved and maintained in 100% ethanol. Lipids were mixed together at a molar % ratio of 40:10:39.8:10:0.2 DLinDMA:PEG-s-DMG:cholesterol:DSPC:SP-DiO₁₈ or 40:10:35.8:9:0.2 with 5% CQ-lipid or 35:10:39.8:0.2 with 5% CQ-lipid. Lipid mixture was added drop-wise to the formulation buffer (50 mM citrate, pH 4.0) to form multi lamellar vesicles (MLV). Large unilamellar vesicles (LUVs) were formed upon extrusion of MLVs through two stacked 80 nm Nuclepore polycarbonate filters using an extruder from Northern Lipids (Vancouver, BC, Canada) at ˜300 psi. In order to encapsulate siRNA, siRNA was added drop-wise to preformed vesicles and incubated at 35° C. for 30 minutes with constant mixing. Removal of ethanol and neutralization of formulation buffer were performed by dialysis in PBS for 16 hours. Distribution of size was determined by dynamic light scattering using a NICOMP 370 particle sizer (Nicomp Particle sizing Inc., Santa Barbara, Calif.). siRNA encapsulation efficiency was determined by removal of free siRNA using VivaPureD MiniH columns (Sartorius Stedim Biotech) from samples collected before and after dialysis. The encapsuted siRNA from eluants were then extracted and quantified at 260 nm. siRNA to lipid ratio was determined by measurement of cholesterol content in vesicles by using the Cholesterol E enzymatic assay from Wako Chemicals USA (Richmond, Va.).

Cells and Reagents

Raw264.7 and LNCaP cells were maintained in DMEM (Invitrogen, Carlsbad, Calif.) supplemented with 10% FBS and 2 mM L-glutamine and RPMI1640 (Invitrogen, Carlsbad, Calif.) supplemented with 5% FBS, respectively at 37° C. with 5% CO₂. Small-molecule drugs used in this study were from the Prestwick collection of the Canadian Chemical Biology Network (CCBN). siRNACy3 and Cy5-siRNA were kindly provided by Alnylam Pharmaceuticals (Boston, Mass.) (Akinc et al., 2008) and GAPDH siRNA (sense strand 5′-UGGCCAAGGUCAUCCAUGAdTdT-3′ and antisense strand 5′-UCAUGGAUGCCUUGGCCAdTdT-3′) (Reynolds et al., 2004) was purchased from Thermo Scientific (Waltham, Mass.). Rabbit polyclonal anti-GAPDH and anti-actin antibodies were purchased from Abcam (Cambridge, Mass.), and HRP-conjugated goat anti-rabbit IgG were purchased from Jackson Immuno Research Laboratories (West Grove, Pa.).

LN Uptake

10,000 Raw264.7 cells were seeded and treated with 1, 5, 10 and 15 μg/mL of siRNACy3-DLinDMA LN for 1, 2, 8 and 24 hours. Cells were fixed with 3% paraformaldehyde in PBS in the presence of Hoescht for 15 minutes. Cells were rinsed and stored in PBS supplemented with calcium chloride and magnesium sulfate. Plates were scanned and analyzed with the Cellomics ArrayScan VTI. For drug confirmation, cells were co-treated with 10 μg/mL of siRNACy3-LN in the presence of specified drug for 24 hours.

Drug Screen

10,000 Raw264.7 cells were seeded and treated with 10 μg/mL of siRNACy3-LN for 24 hours in the presence of ˜10 μM of small molecule drugs pinned from 1000-fold stocks in DMSO using a pinning robot equipped with 0.4 mm pins (BioRobotics, Cambridge, UK). Cells were fixed with 3% paraformaldehyde in PBS in the presence of Hoescht for 15 minutes. Cells were rinsed and stored in PBS supplemented with calcium chloride and magnesium sulfate. Plates were scanned and analyzed with the Cellomics ArrayScan VTI.

Chloroquine Lipid Synthesis

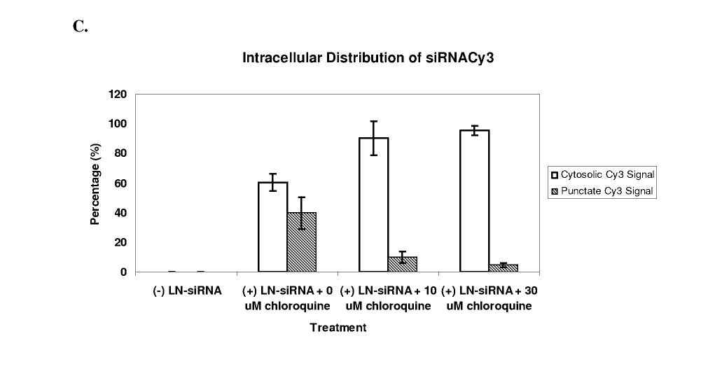

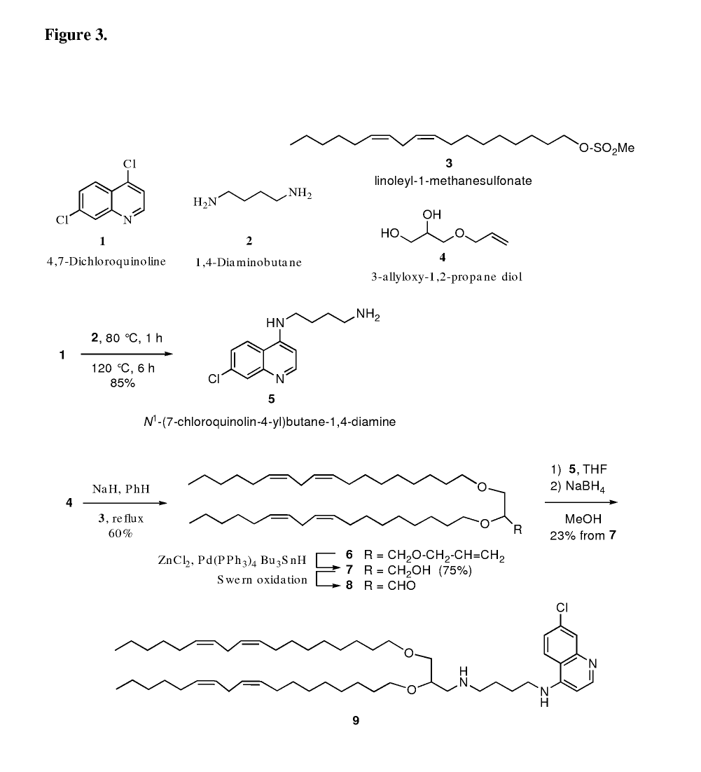

Unless otherwise stated, ¹H (300 MHz) and ¹³C NMR (75 MHz) spectra were recorded at room temperature in CDCl₃ solutions. Chemical shifts are reported in parts per million (ppm) on the δ scale and coupling constants, J, are in hertz (Hz). Multiplicities are reported as “s” (singlet), “d” (doublet), “t” (triplet), “q” (quartet), “m” (multiplet). Mass spectra (m/z) were obtained in the electrospray (ESI) mode. All reagents and solvents were commercial products and used without further purification except THF, Et₂O (both freshly distilled from Na/benzophenone under Ar) and CH₂Cl₂ (freshly distilled from CaH₂ under Ar). Flash chromatography was performed on Silicycle 230-400 mesh silica gel. Analytic and preparative TLC was carried out with Merck silica gel 60 plates with fluorescent indicator. Spots were visualised with UV light. All reactions were performed under dry argon in flame- or oven-dried flasks equipped with Teflon™ stirbars. All flasks were fitted with rubber septa for the introduction of substrates, reagents and solvents via syringe. Compounds were numbered as shown in FIG. 3.

N¹-(7-Chloroquinolin-4-yl)butane-1,4-diamine (compound 5). A well stirred mixture of 4,7-dichloroquinoline (compound 1, 500 mg, 2.52 mmol) and 1,4-diamino butane (compound 2, 222 mg, 2.52 mmol) was heated at 80° C. for 1 h, then the temperature was increased to 120° C. and stirring was continued for an additional 6 h, at which point analysis of the reaction mixture (TLC) indicated that the reaction had proceeded to completion. The mixture was cooled to room temperature and partitioned between aqueous 1N NaOH solution (10 ml) ethyl acetate (50 mL). The organic phase was separated, dried over anhydrous sodium sulfate, filtered, and concentrated in vacuo. Compound 5 was obtained as a pale yellow, amorphous solid (425 mg, 85%) that was used without further purification. ¹H NMR (300 MHz, CDCl₃): 8.50 (d, J=5.47 Hz, 1H), 7.93 (d, J=2.1 Hz, 1H), 7.73 (d, J=9.1 Hz), 7.26-7.33 (dd, J=9.1, 2.1 Hz, 1H), 6.36 (d, J=5.47 Hz, 1H), 3.26-3.32 (m, J=9.5 Hz, 2H), 2.82 (t, J=6.7 Hz, 2H), 1.81-1.88 (m, J=9.5 Hz, 2H), 1.56-1.69 (m, J=9.5 Hz, 1H). ESI-MS: [M+H]⁺250.32.

Compound 6. A solution of 3-allyloxy-1,2-propanediol (compound 4, 792 mg, 6 mmol) in benzene (5 mL) was carefully added to a suspension of NaH (288 mg, 12 mmol) in benzene (10 mL) at 0° C. under an argon atmosphere and the resulting mixture was stirred for 30 minutes. A solution of linoleyl-1-methanesulfonate (compound 3, 2.0 g, 5.8 mmol) in benzene (15 mL) was then added, and the mixture was heated to reflux for 12 h with good stirring. The mixture was then cooled to room temperature and ethanol (5 mL) was carefully added to quench remaining sodium hydride. Water (25 mL) was then added, the organic phase was separated and retained, and the aqueous layer was extracted with ethyl acetate (3×150 mL). The combined extracts were dried over anhydrous sodium sulfate, filtered and concentrated in vacuo. The residue of crude compound 6 was purified by flash column chromatography (5% ethyl acetate/hexanes), to afford 1.7 g (60%) of compound 6 as a colorless viscous oil. ¹H NMR (300 MHz, CDCl₃): 5.80-6.00 (m, 1H), 5.38-5.47 (m, 8H), 5.15-5.29 (m, 2H), 4.00-4.02 (dt, J=5.65 Hz, 2H), 3.39-3.59 (m, 7H), 2.75-2.79 (t, J=6.7 Hz, 4H), 2.02-2.08 (m, 8H), 1.54-1.58 (m, 4H), 1.26-1.38 (m, 32H), 0.86-0.91 (m, 6H). ESI-MS: [M+H]⁺629.7.

Compound 7. Anhydrous ZnCl₂ (1.42 g, 10.5 mmol) was added to a solution of allyl ether compound 6 (1.65 g, 2.6 mmol) in anhydrous THF (25 mL) and the suspension was stirred at room temperature for 15 min. Tetrakis (triphenylphosphine) palladium(0) (250 mg, 0.2 mmol) was added, and stirring was continued for 10 min. Tributyltin hydride (3.05 mL, 10.5 mmol) was slowly added to the above suspension, and the progress of the reaction was followed by TLC. After 30 min, the reaction had proceeded to completion. The mixture was diluted with ethyl acetate (10 mL) and water (5 mL) and acidified to pH 6 with 5% aqueous HCl. The solution was extracted with ethyl acetate (3×50 mL). The combined extracts were dried over anhydrous sodium sulfate, filtered and concentrated in vacuo. The crude product was purified by column chromatography (20% ethyl acetate/hexanes) to afford pure 7 (1.24 g, 75%) as a colorless viscous oil. ¹H NMR (300 MHz, CDCl₃): 5.38-5.47 (m, 8H), 3.41-3.72 (m, 9H), 2.75-2.79 (t, J=6.7 Hz, 4H), 2.02-2.08 (m, 8H), 1.54-1.58 (m, 4H), 1.26-1.38 (m, 32H), 0.86-0.91 (m, 6H). ESI-MS: [M+Na]⁺611.67.

Compound 8. Oxalyl chloride (306 μL, 2.4 mmol) was carefully added to a well stirred, cold (−78° C.) solution of DMSO (265 μL, 3.4 mmol) in dry CH₂Cl₂ (10 mL) under an argon atmosphere. After 30 minutes, a solution of alcohol 7 (1.0 g, 1.7 mmol) in CH₂Cl₂ (10 mL) was added, and stirring was continued for an additional for 30 minutes. Triethylamine (0.5 mL, 4.9 mmol) was then injected, and the mixture was warmed to room temperature. The solution was diluted with water (50 mL) and extracted with CH₂Cl₂ (3×50 mL). The combined extracts were dried over anhydrous sodium sulfate, filtered and concentrated in vacuo. The crude product was filtered through a short plug of silica gel (˜5 g) using 5% ethyl acetate/hexanes. Concentration of the filtrate in vacuo provided crude compound 8, which was used in the next step without further purification. ¹H NMR (300 MHz, CDCl₃): 9.72 (d, 1H), 5.38-5.47 (m, 8H), 3.41-3.72 (m, 7H), 2.75-2.79 (t, J=6.7 Hz, 4H), 2.02-2.08 (m, 8H), 1.54-1.58 (m, 4H), 1.26-1.38 (m, 32H), 0.86-0.91 (m, 6H).

Compound 9. A solution of compound 5 (249 mg, 1 mmol) and compound 8 (586 mg, 1 mmol) in THF (5 mL) was stirred for 12 h at room temperature, then the solvent was removed in vacuo. The residue was diluted with methanol (10 mL), treated with solid sodium borohydride (25 mg, 0.67 mmol), added in portions, and stirred for 6 h. The mixture was concentrated to dryness in vacuo and the residue was acidified with aqueous 1N HCl (5 mL). The resulting aqueous phase was extracted with ethyl acetate (3×25 mL). The combined extracts were dried over anhydrous sodium sulfate, filtered and concentrated in vacuo. The residue was purified by flash column chromatography (5% methanol/CH₂Cl₂), to afford compound 9 (135 mg, 23% from 7) as a yellow viscous oil. ¹H NMR (400 MHz, CDCl₃): 8.49 (d, J=5.47 Hz, 1H), 7.98 (d, J=2.1 Hz, 1H), 7.80 (d, J=9.1, 1H), 7.39 (dd, J=9.1, 2.1 Hz, 1H), 6.38 (d, J=5.47 Hz, 1H), 6.23-6.54 (br, 1H), 5.25-5.45 (m, 8H), 3.59-3.71 (m, 2H), 3.35-3.57 (m, 5H), 3.25-3.35 (m, 2H), 2.60-2.95 (m, 8H), 2.00-2.17 (m, 8H), 1.83-2.00 (m, 2H), 1.7-1.83 (m, 2H), 1.45-1.65 (m, 4H), 1.26-1.38 (m, 32H), 0.89 (t, 6H). ESI-MS: [M+H]⁺820.02.

### Fluorescence Microscopy

Raw264.7 cells grown on glass coverslips were treated with 10 μg/mL Cy5-siRNA-LN/SPDiO or siRNACy3-LN/SPDiO constituted of +/−5% CQ-lipid in the presence of 35 or 40% DLinDMA. The overall uptake of LNs was monitored with SPDiO and the distribution of siRNA was monitored using either Cy5- or Cy3-labelled siRNA. Cells were fixed with 3% paraformaldehyde in PBS in the presence of Hoescht for nuclear staining for 15 minutes. Glass coverslips were mounted onto slides and analyzed by confocal microscopy (Olympus FV1000). Fluorochromes were excited at 488 nm (DiO), 594 nm (Alexa-594), 550 nm (Cy3) and at 633 nm (Cy5) and images were collected sequentially with 60× oil immersion objective lens.

### Results

Small Molecules and Uptake of siRNA into Raw264.7 Cells

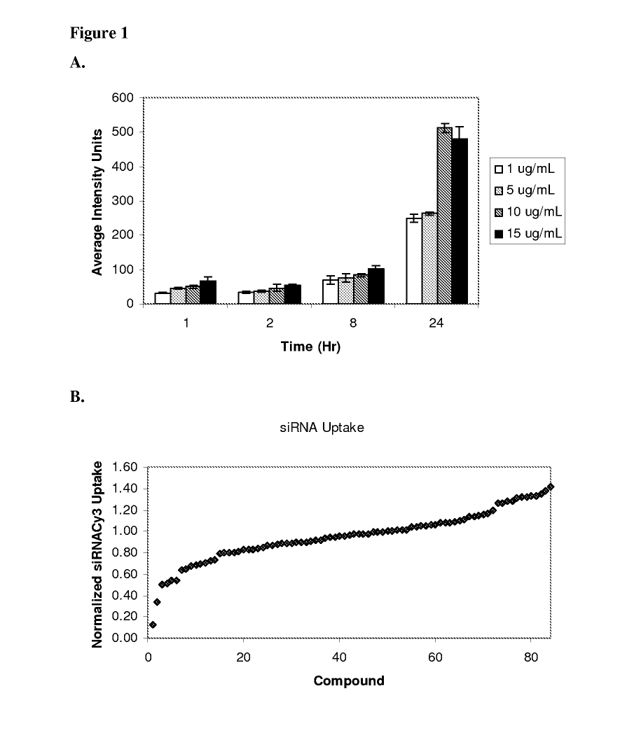

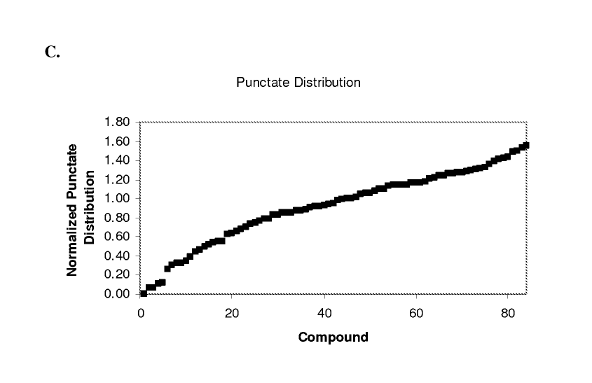

Raw264.7 cells were treated with varying concentration of Cy3-labelled siRNA encapsulated in LNs for 1, 2, 8 and 24 hours. Cellular siRNA was monitored by Cy3 fluorescence. Intracellular siRNACy3 was relatively low at 1 and 2 hours (FIG. 1A), increasing by 8 hours of incubation in a concentration dependent manner. At 24 hours, a significant amount of siRNACy3 accumulated intracellularly with levels of intracellular siRNACy3 at doses of 10 and 15 μg/mL being very similar, suggesting that LN uptake had reached saturation at 10 μg/mL (FIG. 1A).

LNs at a siRNACy3 concentration of 10 μg/mL were used to screen for small compounds that improve intracellular delivery/accumulation of siRNA. A pilot screen consisting of 81 drugs from the Prestwick collection in the CCBN was performed. Upon normalization of Cy3 signal detected in the presence of drugs to that in cells untreated for any drugs, it was observed that the majority of drugs (˜75%) inhibited/hampered the uptake of siRNACy3 (FIG. 1B). However, it is possible that these same drugs may prevent the release/escape of siRNACy3 into the cytosol by preventing fusion of LNs to endosomal membrane resulting in enhanced degradation of siRNAy3 and decreased Cy3 detection (suggested in Lin et al., unpublished). siRNACy3 fluorescence detected as spots or punctate was normalized to total cellular siRNACy3 fluorescence to determine the degree of punctate distribution which could infer accumulation of siRNA in endocytic compartments (FIG. 1C). We observed that approximately half of the small molecules tested increased the punctate distribution of siRNACy3 while the other half increased cytosolic or diffuse distribution of siRNACy3 (FIG. 1C).

The small molecules were ranked according to their normalized intracellular fluorescence. The small molecules that contributed the most to increased intracellular uptake of siRNACy3, as well as enhanced release of siRNACy3 into the cytosol were listed in Table 1. The small molecules that enhance the accumulation of siRNACy3 increased overall siRNACy3 fluorescence by over 28% with a concomitant increase in the level of punctate distribution in respect to untreated cells, inferring that the increased accumulation of siRNACy3 was observed in endosomal compartments (Table 1). This indicates that LNs were taken in more readily in the presence of these drugs but their release/escape into the cytosol was not enhanced, resulting in an accumulation in endocytic compartment. Conversely, drugs that enhanced cytosolic delivery of siRNACy3 impeded the overall uptake of siRNACy3 (Table 1). The relative importance of increased cytosolic distribution and increased overall uptake on siRNA-mediated gene silencing remains to be determined.

Confirmation that Small Molecules Identified Result in Increased Uptake and Delivery

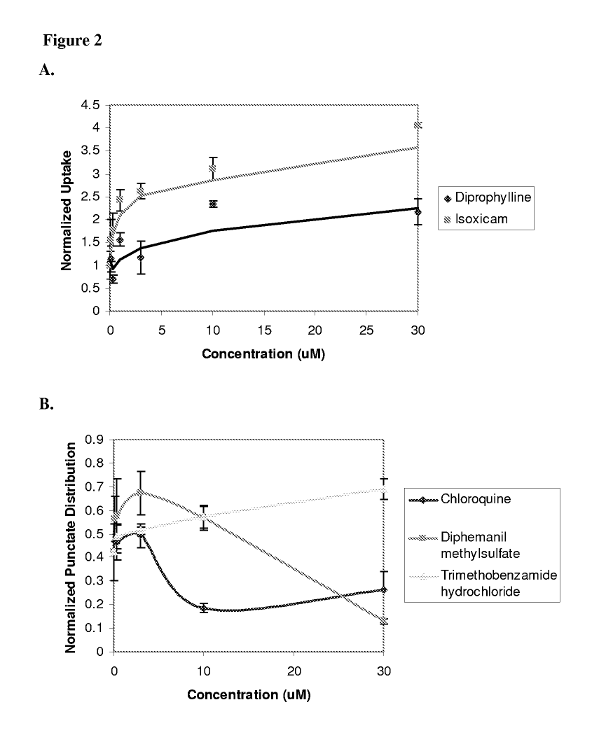

Titration studies were performed to confirm the drugs identified by the Cellomics screen that enhanced the intracellular delivery of siRNA (Table 1). 10,000 Raw 264.7 cells were treated with 10 μg/mL of siRNACy3 encapsulated in DLinDMA LNs in the presence of diprophylline, a drug used to treat pulmonary hypertension (Simons et al., 1975; Cushley and Holgate, 1985; Magnussen et al., 1986), or isoxicam, an anti-inflammatory drug (Zinnes et al., 1982). siRNACy3 fluorescence observed in cells treated with drugs was normalized to that in the absence of drug treatment (FIG. 2A). Both diprophylline and isoxicam upregulated the uptake of siRNACy3. Approximately 2-fold and 3-fold increase in siRNACy3 accumulation was observed for diprophylline and isoxicam, respectively, treated at 30 μM (FIG. 2A). This provides strong evidence that small molecules can enhance the intracellular delivery of LN siRNA.

Increasing the uptake of siRNA can enhance the efficiency of the gene therapy. Alternatively, it is also possible to enhance the siRNA effect by facilitating the escape of siRNA from endocytic compartments into the cytosol. Among the 6 candidates that were identified to decrease the punctate distribution of siRNACy3 (Table 1), 3 candidates (chloroquine, trimethobenzamide hydrochloride and diphemanil methylsulfate) were further analyzed to confirm their function. Trimethobenzamide hydrochloride, an anticholinergic drug to treat nausea (Kolodny, 1960), was a false positive as it showed increased punctate distribution of siRNACy3 with increasing drug concentration (FIG. 2B). Diphemanil methylsulfate, another anticholinergic drug (reviewed in Finkbeiner et al., 1977), only showed enhanced cytosolic distribution of siRNACy3 at 30 μM of treatment (FIG. 2B). In contrast, chloroquine, a widely used malaria drug (Young and Eyles, 1948) showed significant loss of punctate distribution of siRNACy3 at 10 μM of treatment, which confirmed our screening data. Upon further analysis, cotreatment of 10 or 30 μM of chloroquine with siRNACy3 encapsulated LNs showed loss of punctate structure and increased cytosolic distribution of siRNACy3 (FIG. 2C).

Chloroquine Lipid Synthesis

An ideal LN would contain endosomal release agents conjugated to lipid head groups such that the siRNA would be effectively released into the cytosol upon LN internalization. Chloroquine was chosen to conjugate to lipid as it has already been used previously to enhance transfection of cells and gene transfer efficiency (Hasan et al., 1991; Erbacher et al., 1996). The final conjugated lipid utilized in this study, compound 9 in FIG. 3, was made from commercially available, inexpensive compounds 1-4. The synthesis involved the separate preparation of chloroquine-like compound 5 and aldehyde 8, and the union of the two fragments by means of a reductive amination reaction. The various intermediates were purified by flash column chromatography and characterized by ¹H NMR and mass spectrometry.

Chloroquine Lipid Formulations

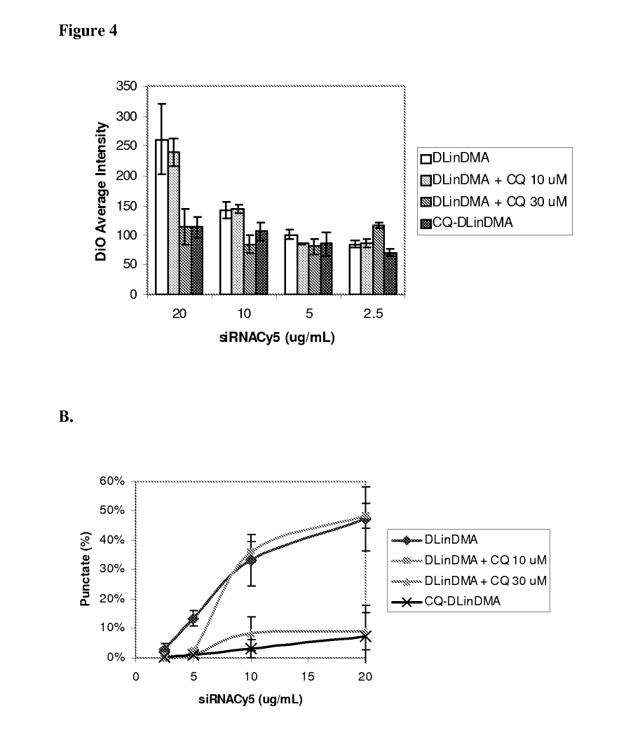

Raw264.7 cells were treated with LNs consisting of 40% DLinDMA in the absence or presence of 10 or 30 μM chloroquine or formulation consisting of 35% DLinDMA with 5% CQ-lipid (˜14.7 μM chloroquine). Total lipid uptake was estimated by cellular SPDiO fluorescence. When cells were treated for 16 hours, the uptake of SPDiO was dependent on the concentration of chloroquine as well as concentration of LNs (FIG. 4A). When Raw264.7 cells were treated only with DLinDMA LNs, the intensity of SPDiO increased in respect to concentration. The presence of 10 μM of chloroquine did not seem to affect the uptake of LNs as it showed no significant difference in comparison to DLinDMA LNs only treatment. Furthermore, Cy5-siRNA punctate levels were also largely indifferent in cells treated with or without 10 μM of chloroquine (FIG. 4B). Interestingly, when cells were treated with LNs in the presence of 30 μM of chloroquine or CQ-lipid-DLinDMA LNs, both SPDiO fluorescence and Cy5-siRNA punctate levels were detected at similar levels (FIGS. 4A and B). Importantly, 30 μM of chloroquine or CQ-lipid caused a significant reduction in Cy5-siRNA punctate levels suggesting that large amount of Cy5-siRNA was released into the cytosol (FIG. 4B). It also indicates that the CQ-lipid can induce similar effects as that of free chloroquine. The Cy5-siRNA distribution at 16 hours was relatively more “punctate” than that at 24 hours (FIGS. 4B and C). Also, cells incubated with 10 μM of chloroquine for 24 hours showed more Cy5-siRNA in the cytosol than those cells at 16 hours suggesting that chloroquine action may take time (FIGS. 4B and C).

To verify the uptake and distribution of LNs and Cy5-siRNA, Raw264.7 cells were incubated with Cy5-siRNA encapsulated in DLinDMA or CQ-lipid-DLinDMA LNs for 16 hours and analyzed by confocal microscopy. The cells were also incubated in the presence of Transferrin-594. SPDiO colocalized partially with Transferrin-594 in cells treated with DLinDMA LNs; however, the colocalization of SPDiO and Transferrin-594 was almost identical in cells incubated with CQ-lipid-DLinDMA LNs (FIG. 4D). Such high degree of colocalization of SPDiO and Trannferrin-594 can be attributed to the lysosomotropic effect of chloroquine. When the same cells were monitored for Cy5-siRNA distribution, the Cy5 signals observed in cells treated with DLinDMA LNs were predominantly punctate while CQ-lipid-DLinDMA LNs induced both punctate and cytosolic distribution of siRNA-Cy5 (FIG. 4E).

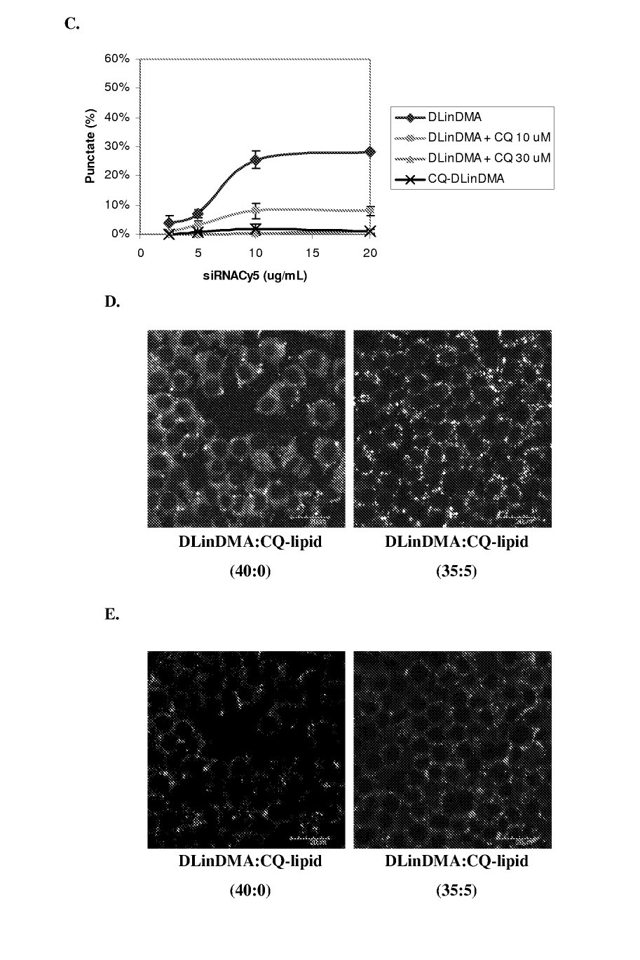

The initial studies with 5% CQ-lipid:35% DLinDMA LN system showed substantially reduced intracellular DiO accumulation at the highest dose in comparison to LN systems comprising of 0% CQ-lipid:40% DLinDMA (FIG. 4A). Additional studies further established that when Raw264.7 cells were treated with LNs consisting of 40% DLinDMA in absence or presence of 5% CQ-lipid the uptake of LN particles showed comparable intracellular SPDiO fluorescence. (FIG. 5A). This indicates that the reduced LN uptake in 5% CQ-lipid:35% DLinDMA LN systems (FIG. 4A) was a result of reduced cationic lipid (DLinDMA) mole % and not due to the chloroquine conjugated lipid. Although uptake was relatively unaffected, the presence of 5% CQ-lipid significantly altered intracellular siRNACy3 distribution (FIG. 5B). In the presence of 5% CQ-lipid, a lowered punctate signal was detected indicating a CQ-lipid assisted release of siRNACy3 into the cytosol (FIGS. 5B and C) consistent with the activity of free chloroquine (FIG. 2B). The effect of CQ-lipid on intracellular distribution of siRNACy3 in Raw264.7 delivered in LN can be clearly seen by confocal microscopy, resulting in enhanced cytosolic distribution in comparison to siRNACy3 delivered in LN consisting of 40% DLinDMA only (FIG. 5C).

### Target Gene Knockdown

Perhaps most importantly, we wanted to determine whether the enhanced cytosolic distribution of siRNACy3 observed with the CQ-lipid correlated to enhanced gene knockdown. Glyceraldehyde 3-phosphate dehydrogenase (GAPDH) is an ubiquitously expressed cytosolic protein and therefore knockdown efficiency of encapsulated siRNA can be tested in different cell lines (Barber et al., 2005). Raw264.7 cells were treated with LN of varying concentration of GAPDH siRNA for 24, 48 and 72 hours. Total protein was isolated and analyzed for GAPDH levels by immunoblotting. While no knockdown of GAPDH was observed at 24 hours, treatment with 20 μg/mL of GAPDH siRNA encapsulated in LN containing 5% CQ-lipid showed decreased GAPDH protein expression (FIG. 5D) by 48 hours. At 72 hours, knockdown of GAPDH was significant in cells treated with either 40% DLinDMA or 40% DLinDMA with 5% CQ-lipid (FIG. 5D) although more pronounced in the presence of 5% CQ-lipid (FIG. 5D). The function of GAPDH siRNA was specific since actin expression was unaltered (FIG. 5D). To confirm the function of the CQ-lipid in the LN-siRNA-mediated gene knockdown, GAPDH silencing was also assessed in LNCaP cells (FIG. 5E). Enhanced knockdown of GAPDH was observed in cells treated with all concentrations of LN containing 5% CQ-lipid as early as 48 hours. More pronounced knockdown effect was observed at 72 hours (FIG. 5E) while expression of actin remained unchanged (FIG. 5E) at all timepoints. Together with the microscopy data, these results indicate that the CQ-lipid enhances gene knockdown by localizing siRNA to the cytosol.

### Discussion

The potential of siRNA as a therapeutic relies on uptake into target cells and delivery into the cytosol. In this report, we wanted to determine whether small molecule drugs can be conjugated to lipids and aid in delivery of siRNA. The advantages of using these compounds include the fact that they are already approved therapeutic drugs and are relatively unlikely to induce immunogenicity. Eighty-one drugs were screened for their effects on LN siRNA uptake and intracellular delivery. Two distinct categories were identified—drugs that enhanced uptake of siRNA and drugs that enhanced cytosolic delivery of siRNA. Diprophylline and isoxicam were shown to increase siRNACy3 uptake but did not increase the cytosolic distribution of siRNACy3 (FIG. 2A). Diprophylline or isoxicam result in an increased intracellular accumulation of siRNACy3 by 2 to 3 fold (FIG. 2A). Diprophylline is a derivative of theophylline (Korzycka and Górska, 2008) while isoxicam is part of the oxicam family of drugs (reviewed in Albengres et al., 1993; Olkkola et al., 1994; Jolliet et al., 1997).

Chloroquine was also identified as a drug that resulted in reduced uptake but increased cytoplasmic delivery. In the absence of chloroquine, a maximum of 25-30% of siRNA remained accumulated in punctate distribution at 24 hours while in the presence of 10 μM chloroquine, ˜10% of the siRNA showed punctate distribution (FIG. 4C). This suggests that chloroquine enhanced the release of siRNA into the cytosol. When cells were treated with 30 μM of chloroquine or using CQ-lipid in the formulation, the siRNA showed reduced punctate accumulation; however, the uptake of LNs was compromised. This observation was not unexpected since chloroquine affects ensodomal pH (Connor and Huang, 1986; Pless and Wellner, 1996) and endocytosis is dependent on proper endosomal pH (Chapman and Munro, 1994). Viral infections in mammalian cells that employ endocytic pathways are also compromised in the presence of chloroquine (Tsiang and Superti, 1984; Zeichhardt et al., 1985; Kooi et al., 1991). Although the uptake of LNs were compromised in the presence of 30 μM chloroquine or using CQ-lipid +35 mole % DLinDMA LNs, the cells showed enhanced cytosolic distribution suggesting the chloroquine moiety or higher chloroquine concentration destabilized the endosomal membrane and assisted the escape of siRNA (Farhood et al., 1995; Guy et al., 1995; Budker et al., 1996). When the ionizable cationic lipid (DLinDMA) was restored to 40 mole % in the presence of 5 mole % CQ-lipid the uptake of LNs did not differ that of DLinDMA LNs (FIG. 5A). Furthermore, the presence of 5 mole % CQ-lipid still induced release of siRNA into the cytosol observed by lower punctate distribution of siRNACy3 (FIG. 5B).

Importantly, siRNA encapsulated in LN with the CQ-lipid showed enhanced gene knockdown in two different cell lines—Raw264.7 and LNCaP. This is encouraging as the CQ-lipid may be widely effective as an agent to enhance gene knockdown in different cells or tissues. We observed that gene knockdown in Raw264.7 cells treated with 20 μg/ml of siRNA encapsulated in LN containing the CQ-lipid occurred 24 hours earlier than in cells incubated with LN without any CQ-lipid. This suggests that CQ-lipid speeds up destabilization of endosomal membrane and therefore the cytosolic delivery of siRNA.

In conclusion, we have developed a fluorescence-based assay to screen for small molecule drugs that enhance cytosolic delivery of siRNA. The molecule identified, chloroquine, can be conjugated to lipid by standard chemistry and the conjugated lipid can be formulated in our LN systems to induce gene knockdown. Furthermore, the CQ-lipid may be used as a viable alternative to co-treatment of free chloroquine to enhance the cytosolic delivery of nucleic acids presented in LN (Farhood et al., 1995; Guy et al., 1995; Budker et al., 1996). Overall, we have showed that novel lipid candidates can be effectively designed using a high throughput screen to identify small molecules that enhance siRNA function.

TABLE 1
Exemplary Compounds Identified In the Screen

| | | |
|---|---|---|
| | Normalized | Normalized |
| | Punctate | siRNA |
| Small Molecule | Distribution | Uptake |

Increased Uptake

| | | |
|---|---|---|
| Levodopa | 1.43 | 1.28 |
| Naphazoline HCl | 1.49 | 1.32 |
| Acetohexamide | 1.48 | 1.32 |
| Diprophylline | 1.55 | 1.38 |
| Isoxicam | 1.53 | 1.42 |

Increased Cytosolic Distribution

| | | |
|---|---|---|
| Azaguanine-8 | 0.07 | 0.54 |
| Isoflupredone acetate | 0.07 | 0.50 |
| Chloroquine | 0.10 | 0.34 |
| Trimethobenzamide HCl | 0.31 | 0.70 |
| Isoxsuprine HCl | 0.33 | 0.51 |
| Diphemanil methylsulfate | 0.34 | 0.70 |

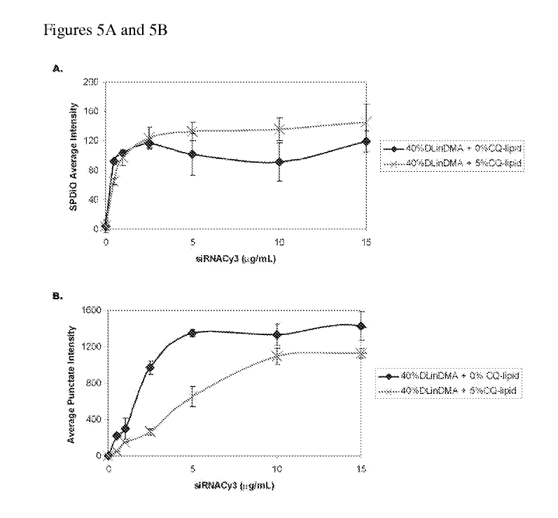

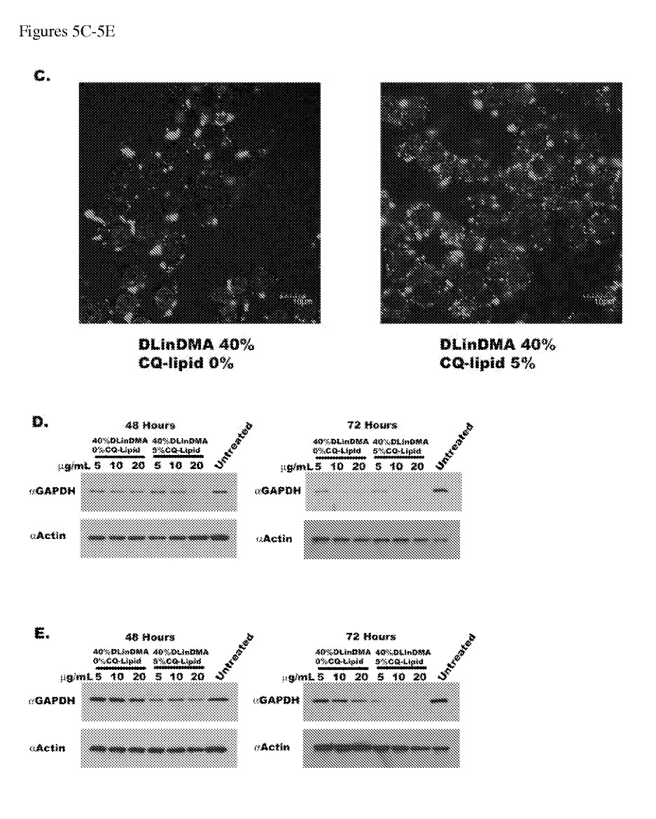

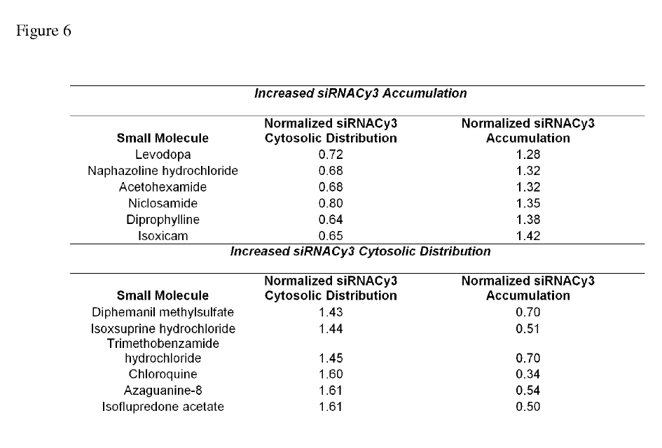

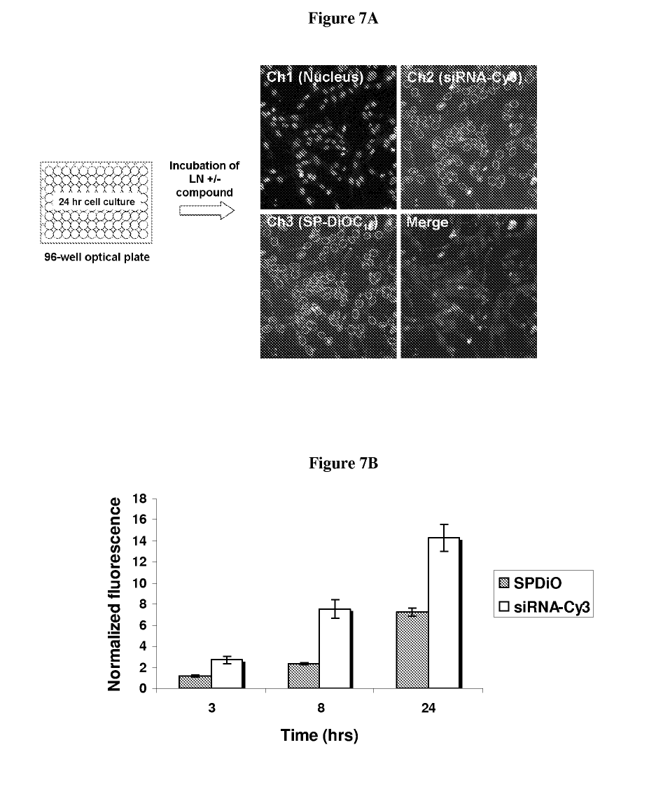

Related data is summarized in FIG. 6, where it is alternatively portrayed as the degree of normalized siRNACy3 cytoplasmic distribution, i.e. diffuse as opposed to punctate.

### Example 2

### Identification of Small Molecules that Enhance Cellular Uptake of Liposomal Particles

Lipid nanoparticle (LNP) formulations of siRNA are now available that can effectively silence genes in hepatocytes following systemic administration. Extension of this ability to other tissues requires the presence of agents on the LNP that promote uptake into component cells. This study was aimed at identifying small molecules that enhance cellular uptake of LNP into a variety of cells and then using these small molecules as LNP-associated ligands to promote LNP uptake. Over 800 small molecules from the Canadian Chemical Biology Network collection of pure chemicals were screened in 6 mammalian cell lines using a Cellomics-based assay to determine their influence on LNP uptake. Molecules that caused the highest uptake of LNP included members of the cardiac glycoside family such as oubain and strophanthidin. Incubation of HeLa cells with LNP GAPDH siRNA systems and oubain resulted in increased LNP uptake and enhanced GAPDH gene silencing effects. A PEG-lipid containing strophanthidin (STR-PEG-lipid) was synthesized as a potential ligand to stimulate LNP uptake into cells. In vitro studies employing HeLa cells showed that internalization of LNP GAPDH siRNA systems and GAPDH silencing was enhanced for LNP siRNA systems containing STR-PEG-lipid as compared to LNP that did not. In vivo studies employing LNP GAPDH siRNA systems containing STR-PEG-lipid show that they are potent systems for silencing GAPDH and, by extension, other genes in kidney tissue following i.v. injection. This is the first time that gene silencing has been observed in non-hepatic tissue following systemic administration of LNP siRNA systems.

Materials and Methods

### Materials

1,2-distearoyl-sn-glycero-3-phosphocholine (DSPC) was purchased from Avanti Polar Lipids (Alabaster, Ala., USA), whereas cholesterol was obtained from Sigma (St Louis, Mo., USA). 1,2-dilinoleyloxy-keto-N,N-dimethyl-3-aminopropane (DLinKDMA) and polyethylene glycol-dimyristol glycerol (PEG-s-DMG) were provided by Tekmira Pharmaceuticals Corporation (Burnaby, BC, Canada). The fluorescently-labelled lipid 3,3′-dioctadecyl-5,5′-di(4-sulfophenyl)oxacarbocyanine, sodium salt (SP-DiO) was purchased from Invitrogen Molecular Probes (Burlington, ON, Canada). The small-molecule library used in this study is from the Canadian Chemical Biology Network (CCBN). Ouabain was purchased from Sigma (St. Louis, Mo., USA).

### Cell Culture

All cell lines were obtained from the American Type Culture Collection (Manassas, Va., USA) and incubated at 37° C. with 5% CO₂ unless indicated otherwise. The human cervix carcinoma cells (HeLa), the mouse macrophages (RAW264.7) and hepatoma cells (Hepal-6) were cultured in Dulbecco's Modified Eagle Medium (DMEM) supplemented with 10% fetal bovine serum (FBS) and 2 mM L-glutamine. Non-essential amino acids were added in medium used to culture Hepal-6 cells. The human breast cancer cells (MDA-MB231) were cultured in DMEM/F12 supplemented with 5% FBS and 2 mM L-glutamine. The canine kidney cells (MDCK) was cultured in minimum essential medium supplemented with 10% FBS, 1 mM sodium pyruvate and 2 mM L-glutamine. The human prostate cancer cells (LNCaP) was cultured in RPMI 1640 medium supplemented with 5% FBS and 2 mM L-glutamine. All cell culture reagents were obtained from Invitrogen (Burlington, ON, Canada). Lipid nanoparticle (LNP) formulations of siRNA are now available that can effectively silence genes in hepatocytes following systemic administration. Extension of this ability to other tissues requires the presence of agents on the LNP that promote uptake into component cells. This study was aimed at identifying small molecules that enhance cellular uptake of LNP into a variety of cells and then using these small molecules as LNP-associated ligands to promote LNP uptake. Over 800 small molecules from the Canadian Chemical Biology Network collection of pure chemicals were screened in 6 mammalian cell lines using a Cellomics-based assay to determine their influence on LNP uptake. Molecules that caused the highest uptake of LNP included members of the cardiac glycoside family such as oubain and strophanthidin. Incubation of HeLa cells with LNP GAPDH siRNA systems and oubain resulted in increased LNP uptake and enhanced GAPDH gene silencing effects. A PEG-lipid containing strophanthidin (STR-PEG-lipid) was synthesized as a potential ligand to stimulate LNP uptake into cells. In vitro studies employing HeLa cells showed that internalization of LNP GAPDH siRNA systems and GAPDH silencing was enhanced for LNP siRNA systems containing STR-PEG-lipid as compared to LNP that did not. In vivo studies employing LNP GAPDH siRNA systems containing STR-PEG-lipid show that they are potent systems for silencing GAPDH and, by extension, other genes in kidney tissue following i.v. injection. This is the first time that gene silencing has been observed in non-hepatic tissue following systemic administration of LNP siRNA systems.

Preparation of siRNA-LNP

All lipid stocks were prepared in 100% ethanol. siRNA-Cy3 targeting mouse factor VII mRNA was obtained from Alnylam Pharmaceuticals (Cambridge, Mass., USA). siRNA (5′-TGGCCAAGGTCATCCATGA-3′) directed to glyceraldehyde 3-phosphate dehydrogenase (siGAPDH) was purchased from Dharmacon (Thermo Scientific, Pittsburgh, Pa., USA). siRNA with a random sequence of low GC content (siScramble) was purchased from Invitrogen (Burlington, ON, Canada). siRNA-Cy3 was encapsulated in LNP consisting of DLinKDMA/DSPC/cholesterol/PEG-s-DMG/SPDiO at a molar ratio of 40/10/39.8/10/0.2 whereas siGAPDH and siScramble were encapsulated at molar ratio of 40/18.8/40/1/0.2 using an ethanol dialysis procedure as previously described with modification ((Maurer et al., 2001); (Jeffs et al., 2005)). Briefly, lipids were mixed together in 30% ethanol and the mixture was slowly added to 50 mM citrate or acetate buffer, pH 4.0 under rapid vortexing followed by extrusion through two stacked of 80 nm polycarbonate filters (5 passes) at ˜300 psi. The siRNA solution was then slowly added to the liposome dispersion equivalent of ten times the amount of siRNA under vortexing. The mixture was subsequently incubated at 31° C. for minutes with constant mixing and dialyzed twice in 1×PBS for 18 h to remove most of the ethanol. Mean vesicle diameter was determined using a submicron quasi-elastic light scattering particle sizer (Nicomp, Santa Barbara, Calif., USA). Cholesterol concentration in LNP was determined by using the Cholesterol E enzymatic assay (Wako Chemicals, Richmond, Va., USA) and was used to infer total lipid concentration in LNP. Removal of free siRNA was performed by using VivaPureD MiniH columns (Sartorius Stedim Biotech GmbH, Goettingen, Germany). The eluants were then lysed and siRNA was quantified by measuring absorbance at OD₂₆₀.

### Small Molecules Treatment on 96-Well Plate

All cell lines were seeded at 5000 to 20,000 cells/well of 96-well ViewPlate (PerkinElmer, Shelton, Conn., USA) in 100 μl of medium and were allowed to grow overnight. Fresh medium containing 5 or 10 μg/ml of siRNA encapsulated in LNP was added the next day. Small molecules were either added manually or pinned from 1000-fold stocks in DMSO using a pinning robot equipped with 0.4 mm pins (BioRobotics, Cambridge, UK). Cells were incubated for 24 h. Cells were then washed once in PBSCM (1×PBS containing 1 μM MgCl₂ and 0.1 μM CaCl₂), fixed in 3% paraformaldehyde containing Hoescht's stain for 15 min, washed once in PBSCM and stored in 100 μl of PBSCM.

### Imaging and Image Analysis

Plates were imaged using a Cellomics Arrayscan VTI HCS Reader (Thermo Scientific, Pittsburgh, Pa., USA). Images were acquired using a 20× PlanFluor objective and a XF93 filter set. Object identification and image analysis were performed using the Cellomics Compartmental Analysis algorithm. Cellular SPDiO and siRNA-Cy3 fluorescence intensities were measured for a minimum of 400 cells and the average pixel intensity was examined. For confocal microscopy, cells grown on glass coverslips were washed once in 1×PBS, fixed in 3% paraformaldehyde containing Hoechst's stain for 15 min, washed again and mounted on slides. Images were captured on an Olympus FV1000 (Olympus, Center Valley, Pa., USA) laser scanning microscope and cellular SPDiO fluorescence intensity was analyzed using ImageJ (NIH, http://rsb.info.nih.gov/ij/).

### Immunoblotting

HeLa cells were plated in twelve-well plates for indicated times. They were then washed in PBS and extracted in RIPA buffer (1% NP-40 and 0.5% Deoxycholic in 1×PBS) supplemented with protease inhibitor tablets (Roche Diagnostics). Total protein quantified by the Bradford Assay was analyzed by immunoblotting using antibodies to GAPDH, β-actin (Abcam, Cambridge, Mass.) or ATP1A1 ((Millipore, Billerica, Mass.). Antigen-antibody complexes in immunoblots were detected using Millipore Immobilon Western Chemiluminescent HRP Substrate (Millipore, Billerica, Mass.).

### Synthesis of STR-PEG

Strophanthidin was obtained from MP Biomedicals. DSPE-PEG-NH₂ was obtained from Avanti Polar Lipids. 2,4,6-Trichlorobenzoyl chloride was obtained from TCI America. Reagent grade triethylamine (Et₃N) was stored over potassium hydroxide pellets. All other reagents were obtained from Sigma Aldrich or Fisher and used as received. Dry solvents were distilled under an atmosphere of nitrogen from standard drying agents: tetrahydrofuran (THF) from sodium benzophenone ketyl; dichloromethane (CH₂Cl₂) and pyridine from calcium hydride.

All reactions were performed using flame- or oven-dried glassware with Teflon™ under an atmosphere of argon. Standard syringe-septum cap techniques were employed for the transfer of all reagents. Analytical thin layer chromatography (TLC) was carried out on Merck silica gel 60 plates with fluorescent indicator and spots were visualized under ultraviolet light or by staining with iodine, potassium permanganate, p-anisaldehyde or ninhydrin. Column chromatography was carried out on Silicycle silica gel 40-63 μm (230-400 mesh) and preparative TLC was carried out on Analtech UNIPLATE glass-backed plates (silica gel GF, 1000 μm coating, 20×20 cm) with UV254 preparative layer. NMR signals are described as follows: s, singlet; d, doublet; t, triplet; m, multiplet; br, broad. Chemical shifts of NMR spectra calibrated to residual solvent signal: CDCl₃ (¹H, 7.26 ppm and ¹³C, 77.0 ppm); pyridine-d₅ (¹H, 8.74 ppm and ¹³C, 150.3 ppm). Melting points are uncorrected. Ultra performance liquid chromatography (HPLC) using evaporative light scattering detection (ELSD) performed by Centre for Drug Research & Development (Vancouver, BC).

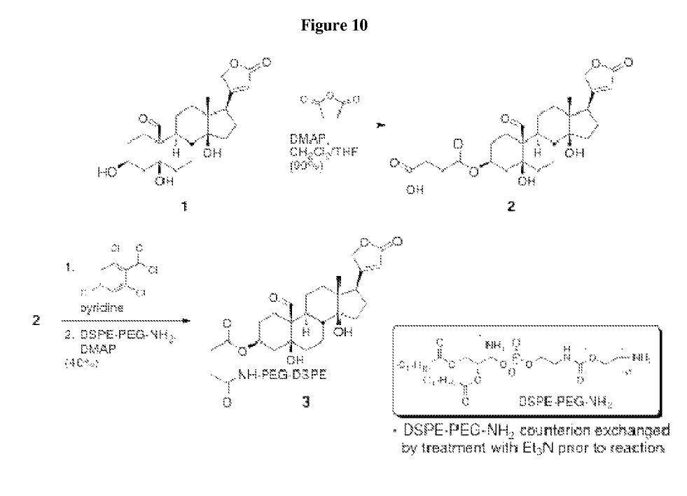

Synthesis scheme is outlined in FIG. 10. To synthesize strophanthidin 3-succinate (2), strophanthidin (1) (303 mg, 0.75 mmol), succinic anhydride (375 mg, 3.75 mmol) and 4-dimethylaminopyridine (458 mg, 3.75 mmol) were added to a dry round bottom flask under argon, followed by 2:1 CH₂Cl₂/THF (7.5 ml) and the mixture vigorously stirred. After 13 hours, the reaction mixture, which now contained a white precipitate, was diluted with CHCl₃ and transferred to a separatory funnel. The organics were washed with aqueous 1 M HCl (2×10 ml), water (2×10 ml), brine (1×10 ml), dried over anhydrous sodium sulfate and concentrated to dryness on a rotary evaporator to yield the crude as a white solid. Recrystallization from MeOH/CHCl₃/hexanes at −20° C. furnished strophanthidin succinate as small, colourless crystals (318 mg, 84%) in two crops: R_{f} 0.13 (silica, 90:10 CHCl₃/MeOH); HPLC-ELSD t_{R} 0.31 min (100% peak area); mp 234-236° C. (MeOH/CHCl₃/hexanes) with darkening; ¹H NMR (300 MHz, pyridine-d₅) δ 10.47 (s, 1H), 6.15 (s, 1H), 5.38 (s, 1H), 5.32 (d, 2H, J=18.3 Hz), 5.06 (d, J=18.3 Hz), 2.95-2.63 (m, 7H), 2.53-2.27 (m, 4H), 2.19-1.65 (m, 10H), 1.62-1.29 (m, 5H), 1.02 (s, 3H) ppm; ¹³C NMR (75.5 MHz, pyridine-d₅) δ 208.8, 176.2, 175.6, 175.0, 172.9, 118.3, 84.8, 74.2, 73.1, 70.6, 55.3, 51.6, 50.3, 42.4, 40.0, 39.6, 38.5, 37.1, 32.7, 30.8, 30.4, 27.7, 25.6, 24.6, 23.0, 19.3, 16.4 ppm; MS (ES) 527 (M+Na, 42), 130 (68), 123 (100).

To synthesize strophanthidin-PEG-DSPE conjugate (STR-PEG) (3), 2,4,6-Trichlorobenzoyl chloride (8 μl, 5.38×10⁻² mmol) was added to a room temperature solution of strophanthidin 3-succinate (27 mg, 5.38×10⁻² mmol) in pyridine (100 μl), followed by Et₃N (15 μl, 0.11 mmol), which caused a colour change to brown. After stirring the above solution for 25 minutes, a solution of DSPE-PEG-NH₂ (30 mg, 1.08×10⁻² mmol; treated prior with excess Et₃N in CH₂Cl₂ and then rotary evaporated to exchange H₄N⁺ counterion for Et₃HN⁺ and repeated once more) in pyridine was added, followed by solid 4-dimethylaminopyridine (7 mg, 5.38×10⁻² mmol), and the resulting mixture was stirred at room temperature for 16 h. The reaction mixture was diluted with CHCl₃, washed with water (2×3 ml), brine (1×3 ml), dried over sodium sulfate and concentrated to dryness on a rotary evaporator. The brown residue was dissolved in CHCl₃ and loaded on a column for chromatography (silica, 95:5→90:10→80:20 CHCl₃/MeOH) to give strophanthidin-PEG-DSPE, along with some minor impurities. The conjugate was purified further by preparative TLC, eluting with 80:20 CHCl₃/MeOH, to furnish the strophanthidin-PEG-DSPE conjugate (22 mg, 67%) as a pale yellow oil: R_{f} 0.38 (silica, 80:20 CHCl₃/MeOH); HPLC-ELSD t_{R} 1.51 min (95.5% peak area); ¹H NMR (300 MHz, CDCl₃) δ 10.05 (s, 1H), 6.72 (br s, 1H), 5.86 (br s, 2H), 5.18 (s, 1H), 4.95 (d, 1H, J=18.0 Hz), 4.78 (d, 1H, J=18.0 Hz), 4.41-4.06 (m, 3H), 4.04-3.23 (m, 180H), 2.90-2.39 (m, 12H), 2.35-1.88 (m, 14H), 1.87-1.42 (m, 16H), 1.24 (br s, 54H), 0.88 (br s, 9H) ppm; MALDI-TOF (2,5-DHB matrix) calculated for C₁₅₉H₂₉₅N₂O₆₂P⁺ 3258, found 3263.

### In Vivo Animal Studies

Eight-week-old female C57Bl/6 mice were obtained from Charles River Laboratories (Wilmington, Mass.). Mice were housed and handled with protocols approved by the Canadian Council on Animal Care. LNP systems were filter-sterilized, diluted to the appropriate concentrations in sterile PBS immediately before use and administered systemically via the tail vein in a total volume of 10 ml/Kg corresponding to 2.5 mg/Kg siRNA equivalence. After 4 days, animals were sacrificed and tissues were harvested and stored in RNAlater (Ambion, Applied Biosystems, Carlsbad, Calif.) at −20 degrees. To extract total RNA, less than 100 mg of tissue was homogenized using the FastPrep-24 (MP Biomedicals, Solon, Ohio) with one ¼″ ceramic sphere in 1 mL of Trizol (Invitrogen, Burlington, ON, Canada) at speed setting 5.5 twice for 15 seconds. Debris was removed by centrifugation and 0.3 mL chloroform was added to the supernatant. Following centrifugation, 0.5 mL isopropanol was added to the aqueous phase and the resulting RNA precipitate was washed with 95% ethanol and resuspended in water. To quantify the reduction of mouse GAPDH mRNA, 1 μg of total RNA was reverse transcribed into cDNA using the High Capacity cDNA Reverse Transcription Kit and quantitative real-time PCR was performed using the ABI 7900HT Fast Real-Time PCR System. Cycle thresholds of target GAPDH and reference 18S were determined using the instrument's software SDS2.3 and RQ Manager (Applied Biosystems, Carlsbad, Calif.). Detection was made by measuring the fluorescence of SYBR green in the reaction mixture when bound to the double-stranded DNA product. For this purpose, EXPRESS SYBR GreenER qPCR Supermix (Invitrogen, Burlington, ON, Canada) was used and each reaction was composed of 2 uL cDNA, 0.4 μM of the primer-pair, 10 uL SYBR supermix, 0.4 uL ROX internal reference dye, and water up to 20 uL. The forward and reverse oligonucleotide primers for the GAPDH target gene were GGCATTGCTCTCAATGACAA and TTCTTACTCCTTGGAGGCCA and for the 18S reference gene were AAACGGCTACCACATCCAAG and CCTCCAATGGATCCTCGTTA. The ratio of target GAPDH to reference 18S mRNA was calculated according to the 2^{-ΔΔCT} method and manufacturer's instructions. mRNA levels are expressed as a group averaged relative quantity normalized to the PBS control group.

### Results and Discussion

### Quantitative LNP Uptake Assay Using Cellomics Arrayscan

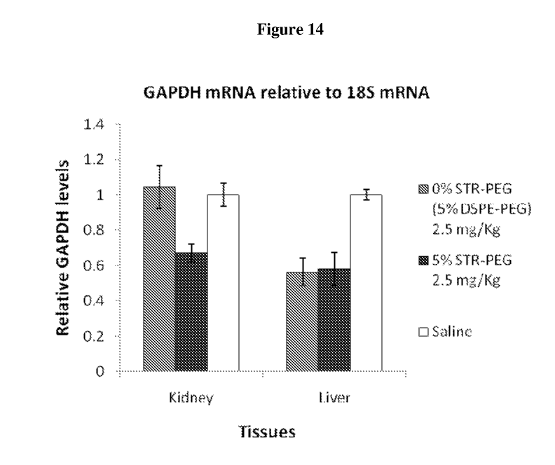

We performed a quantitative LNP uptake assay to measure levels of LNP in cells using fluorescent probes for nuclei (Ch1), siRNA (Ch2) and lipid (Ch3), and the automated fluorescence microscope Cellomics ArrayScan (FIG. 14 FIG. 14A). The LNP formulation used in this study is a potent gene silencing delivery system in hepatocytes in vivo (Semple et al., 2009). HeLa cells were incubated overnight in 96-well optical plates. Fluorescently-labelled LNP was added to cells the next day and incubated for 3 h, 8 h and 24 h (FIG. 14 FIG. 14B). Cells were then fixed and washed before scanning. Hoechst's stain which stains the cell nuclei was used to form the nuclear mask (blue line in Ch1, FIG. 14 FIG. 14A) to identify valid objects or cells. Cellular siRNA was monitored by the fluorophores Cy3 which was conjugated at the 3′ end of the siRNA sense strand (Ch2).

Cellular lipid content was reported by the fluorescent lipid, SPDiO (Ch3). A cellular mask (green line in Ch2 or Ch3, FIG. 14 FIG. 14A) which was slightly larger than the nuclear mask but stayed within the cell boundary was used to delineate the area from which Ch2 or Ch3 cytological features were measured. At least 400 cells were scored per well. Due to different cell morphology exhibited by each cell line, we focused our analysis using one cytological feature which was the average fluorescence intensity per pixel in each channel. Fluorescence values were normalized to that of cells untreated with any LNP (FIG. 14 FIG. 14B). We observed that there was a progressive increase in cellular SPDiO or siRNA-Cy3 fluorescence suggesting that LNP was taken up by cells in increasing amount over time. We therefore used this quantitative fluorescence assay to identify small compounds that enhance targeting of LNP to specific cells.

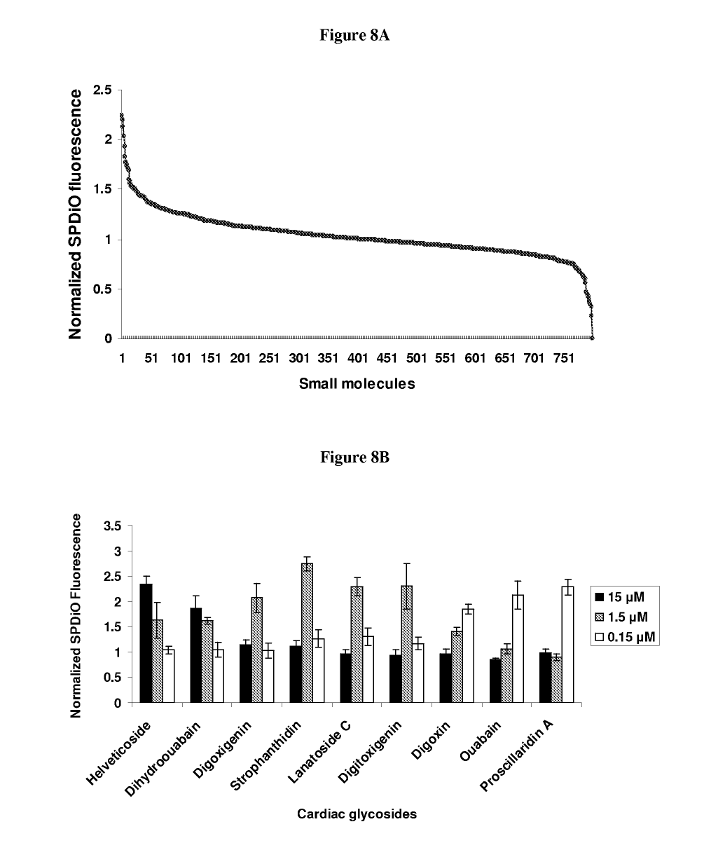

We employed 6 cell lines, HeLa, MDCK, Raw264.7, MDA-MB231, Hepa1-6 and LNCaP to screen 800 compounds in the Canadian Chemical Biology Network collection of small molecules. Compounds were pinned into the 96-well optical plates containing cells and LNP using a pinning robot. Approximately 7.5 μM to 10 μM of each compound were transferred by each pin into each well. For the purpose of this report, only results for HeLa cells were analyzed and shown (FIG. 8A). Approximately half of the molecules led to enhanced LNP uptake with various levels in HeLa cells whereas the others showed decreased LNP uptake. It is believed that LNP enters the cell through the endocytic pathway (Watson et al., 2005; Zelphati and Szoka, 1996) consistent with recent results from this laboratory that LNP enters the cell by endocytosis and colocalizes with early endosomal marker in both Raw264.7 and primary antigen presenting cells (Basha et al., 2009; Lin et al., 2009). We hope to find molecules that enhance LNP uptake by acting on the endocytosis pathway. Interestingly, among the 7 molecules that led to the most cellular SPDiO fluorescence, there are three molecules that belong to the family of cardiac glycosides.

### Cardiac Glycosides Enhance LNP Uptake

Cardiac glycosides are a diverse family of naturally derived molecules. Members of this family have been used to treat heart failure for many years (Schoner and Scheiner-Bobis, 2007). They bind to and inhibit Na⁺/K⁺-ATPase on the plasma membrane thereby leading to the increase of intracellular Ca²⁺ concentration and enhanced cardiac contractility. The binding site has been determined to be at the extracellular side of the α-subunit of the enzyme. It has been suggested that binding of cardiac glycosides to the ATPases paralyzes the enzyme's extracellular domain and therefore affects the catalytic activity of the enzyme and ion transport. Recent studies have demonstrated another role for Na⁺/K⁺-ATPase as signal transducer (Aizman and Aperia, 2003; Kometiani et al., 2005; Xie and Askari, 2002). Binding of cardiac glycosides to the ATPase elicits interaction of the ATPase with neighboring membrane proteins leading to organized cytosolic cascades of signaling proteins to send messages to the intracellular organelles. It has also been shown that binding of ouabain, a member of cardiac glycosides, induces endocytosis of the Na⁺/K⁺-ATPase via a caveolin- and clathrin-dependent mechanism (Liu et al., 2004; Liu et al., 2005).

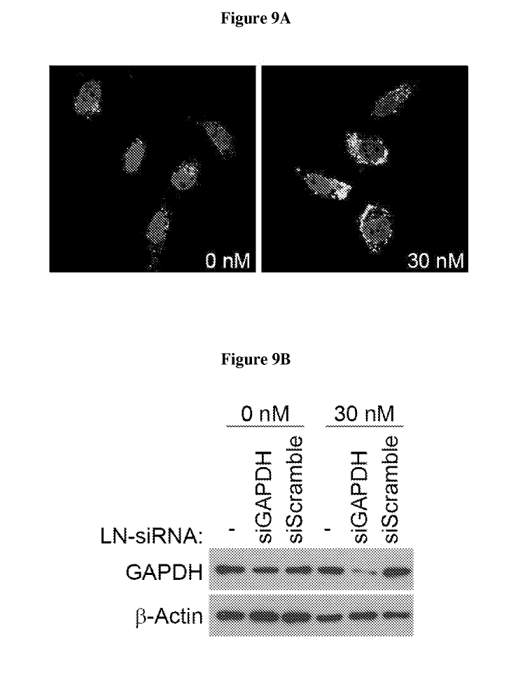

Interestingly, members of the cardiac glycoside family possess different binding affinities and inhibitory effects to Na⁺/K⁺-ATPase (Paula et al., 2005). We tested LNP uptake in HeLa cells in the presence of 9 cardiac glycosides at 3 concentrations, 0.05 μM, 0.15 μM and 1.5 μM for 24 h (FIG. 9). Their relative binding affinity to Na+/K+-ATPase have been shown elsewhere, notably helveticoside<dihydroouabain<digoxigenin<strophanthidin<lanatoside C<digitoxigenin<digoxin<ouabain<proscillaridin A (Paula et al., 2005). Helveticoside, being the weakest binder to the Na⁺/K⁺-ATPase, showed enhanced LNP uptake at a higher concentration. In contrast, Proscillaridin A which is the strongest binder among the 9 molecules tested, required the least amount to cause an increased LNP uptake (FIG. 8B). The levels of LNP uptake resulted from different cardiac glycosides seems to be consistent with their relative binding affinities to the ATPase. It is likely that molecules that have a high affinity to the ATPase would induce more internalization of the ATPase and therefore LNP that are close to the plasma membrane and the ATPase get endocytosed into the same endocytic vesicle.

### Ouabain Induce LNP Uptake and GAPDH Gene Knockdown

Incubation of LLC-PK1 cells with 50 nM of ouabain for 12 hrs induces endocytosis of the Na⁺/K⁺-ATPase to endosomes (Liu et al., 2004; Liu et al., 2005). We confirmed the uptake of LNP in HeLa cells incubated with 30 nM of ouabain for 24 hrs by confocal microscopy (FIG. 9A). Quantification of cellular SPDiO fluorescence showed that cells treated with 30 nM of ouabain contained the most LNP (2.5 times more than untreated cells). SPDiO fluorescence appeared to be localized in punctate structures characteristic of endocytic compartments. Subcellular fractionation showed that a significant amount of the Na⁺/K⁺-ATPase was enriched in the endosomal fraction in cells treated with 30 nM of ouabain (data not shown). These results indicate that our LNP systems were likely endocytosed together with the Na⁺/K⁺-ATPase due to the effect of ouabain.

Knockdown effect of our LNP systems in the presence or absence of ouabain was also examined (FIG. 9B). GAPDH was chosen as a target gene since it is ubiquitously expressed at high levels in all cell types. siGAPDH or negative control siScramble was encapsulated in our LNP systems and incubated with cells for 24 hrs in the presence or absence of ouabain. Cells were further incubated in plain medium for 48 hrs before protein expression was analyzed. We observed that cells incubated continuously with ouabain for longer than 24 hrs were unhealthy (data not shown). Expression of GAPDH was substantially reduced only in cells treated with siGAPDH-LNP and ouabain. The gene knockdown effect was strictly due to enhanced LNP uptake caused by ouabain as we did not observe changes in levels of GAPDH in cells not treated with ouabain or treated with siScramble-LNP (FIG. 9B). It is noteworthy that LNP encapsulating siGAPDH or siScramble contained 1 mol % of PEG-s-DMG. This reduced amount of PEG lipid seemed to be essential for gene knockdown to occur because LNP made with 10 mol % of PEG-s-DMG showed enhanced uptake but no gene knockdown effect (data not shown). Also, a lower mol % of PEG lipid would better mimic in vivo situations where the PEG lipid exchanges out of the LNP systems during circulation (Mori et al., 1998; Palmer et al., 2003). We have also observed that ouabain enhanced GAPDH knockdown in LNCaP cells (data not shown). This is not surprising as the Na⁺/K⁺-ATPase is expressed in almost all tissues albeit at variable levels (Su et al., 2004). It would be interesting to examine ouabain induced LNP uptake and gene knockdown effect in cells of other than human origin. It has been reported that different species have different sensitivities to the same cardiac glycoside because of the presence of different Na⁺/K⁺-ATPase isoforms (Antonipillai et al., 1996; Sweadner, 1989). For example, human α₁-isoform of Na⁺/K⁺-ATPase is highly sensitive to ouabain, whereas the rodent α₁-isoform is 1000-fold less sensitive (Antonipillai et al., 1996). This might be an important consideration for in vivo experiments where rodents are likely used.

### Targeting Lipid Increase LNP Uptake and Gene Knockdown In Vitro

Since free cardiac glycoside was able to enhance LNP uptake and gene knockdown in HeLa cells, we hypothesized that a targeting lipid containing a cardiac glycoside in its headgroup could target more LNP to cells that express Na⁺/K⁺-ATPase. Strophanthidin was chosen to conjugate to the distal end of a 2,000 MW polyethylene glycol (PEG) lipid with distearyl (C18) fatty acid chain (STR-PEG) providing a stable hydrophobic anchor for the targeting PEG-lipid to our LNP (FIG. 10). STR-PEG was successfully formulated into LNP using our standard protocol to produce particles of approximately 80 nm.

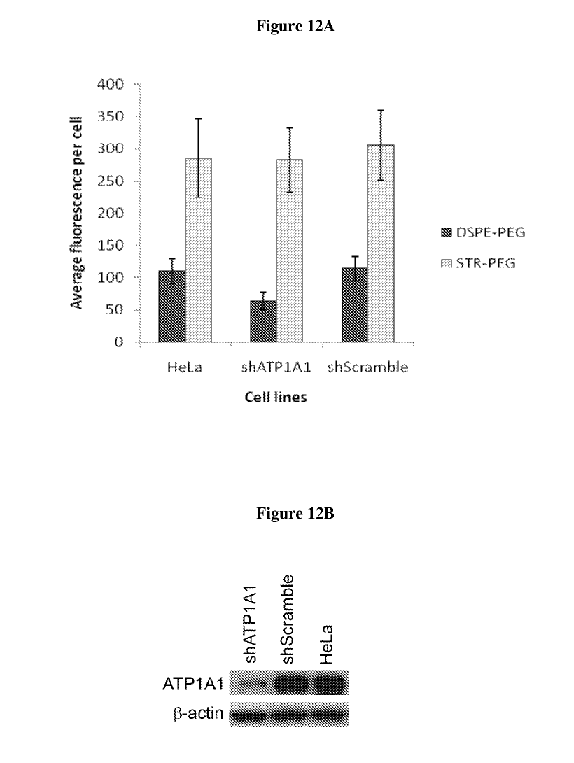

Uptake of LNP containing STR-PEG was examined. Cells were treated with LNP with or without the targeting lipid for 24 hrs and analyzed by confocal microscopy (FIG. 12A). We observed that HeLa cells contained 2.6 times more STR-PEG-LNP than control LNP (DSPE-PEG-LNP) suggesting that the targeting lipid induced more endocytosis of LNP into cells.

Although the literature indicates that the target for cardiac glycosides is the Na⁺/K⁺-ATPase, we investigated whether LNP uptake is dependent on the Na⁺/K⁺-ATPase. Stable cell lines expressing shRNA targeted to ATP1A1 (shATP1A1), the α₁-isoform of Na⁺/K⁺-ATPase, or a negative control sequence (shScramble) were constructed. The levels of ATP1A1 were significantly lower in shATP1A1 cells than shScramble cells or wild-type cells (FIG. 12B). DSPE-PEG-LNP uptake was reduced by ˜42% in shATP1 cells but not in shScramble cells (FIG. 12A). Interestingly, uptake of STR-PEG-LNP was unaffected in shAP1A1 cells even though the levels of Na⁺/K⁺-ATPase was low suggesting that the residual Na⁺/K⁺-ATPase was still active and our targeting lipid was very effective in inducing endocytosis of LNP. These results indicate that LNP uptake in HeLa cells is dependent on the Na⁺/K⁺-ATPase; however, it is unclear whether other uptake mechanisms are involved at this point.

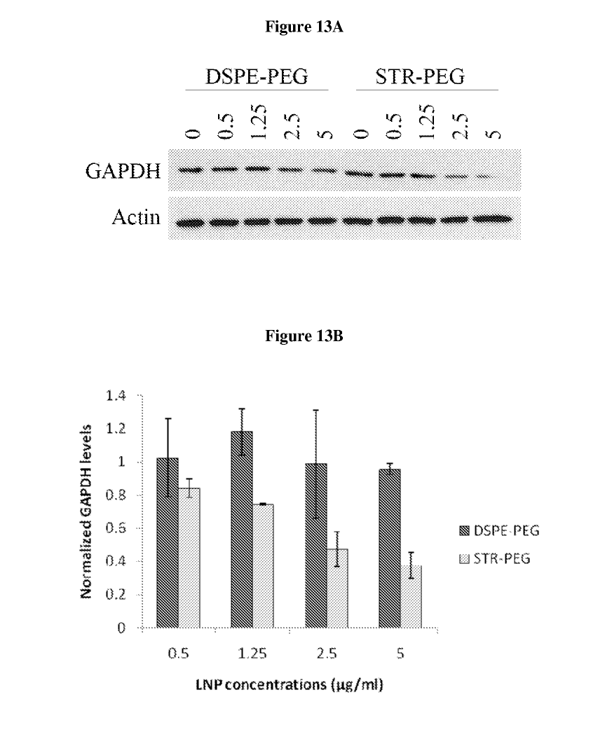

Like using the free ouabain, we expected that our targeted LNP systems could enhance target gene knockdown due to more LNP being internalized into cells. HeLa cells were treated with various concentrations of STR-PEG-LNP or DSPE-PEG-LNP encapsulating siGAPDH for 72 hrs. GAPDH levels were analyzed by western blotting (FIG. 13A) and quantified (FIG. 13B). STR-PEG-LNP was able to induce ˜60% of GAPDH knockdown at 2.5 and 5 μg/ml of siRNA (FIG. 13B). However, cells treated with DSPE-PEG-LNP did not show reduction of GAPDH at any concentrations tested.

### Knockdown of GAPDH in Mouse Kidney

Delivering of LNP to tissues outside the liver has always been a challenge due to majority of LNP being absorbed by the liver. Our targeted LNP could potentially reach tissues other than the liver and give gene knockdown for two reasons. First, these particles contain long chain (C18) PEG that allows LNP to circulate in the blood for a longer period of time. Secondly, the targeting ligand should induce more LNP uptake in cells that express high levels of the receptor. As a proof-of-principle experiment, we examined GAPDH knockdown in both the liver and kidney of mice. Kidney was chosen as it has been shown to express high levels of Na⁺/K⁺-ATPase (Su et al., 2004). Liver, on the other hand, expresses approximately 10 fold less Na⁺/K⁺-ATPase than the kidney but since our LNP systems are expected to accumulate at the liver, knockdown is also expected. C56Bl/6 mice were injected intravenously with 2.5 mg/Kg of LNP containing 5% STR-PEG or 5% DSPE-PEG. Tissues were harvested 96 hours post-injection and GAPDH levels were analyzed by quantitative real-time PCR (FIG. 14). Knockdown of GAPDH mRNA was observed in the liver using either targeted or non-targeted LNP. The targeting lipid seemed to have no benefit in gene knockdown in the liver. This is not surprising as LNP is taken up in the liver predominantly through a mechanism involving apolipoprotein E (ApoE) (Akinc et al., 2010). Interestingly, ˜33% of GAPDH mRNA was reduced in the kidney using STR-PEG-LNP while no GAPDH knockdown was achieved without the targeting ligand (FIG. 14).

These results indicate that cardiac glycosides promote LNP uptake by stimulating endocytosis of the Na⁺/K⁺-ATPase. Ouabain or other cardiac glycosides certainly increase accumulation of LNP in cells. From our uptake and knockdown experiments, it seems that a certain amount of LNP has to be inside the cells for efficient gene knockdown to occur. Whether it is the critical amount of siRNA or membrane destabilizing cationic lipid that leads to gene knockdown remains to be determined.

Our screen and quantitative assay constitutes a novel approach to identify small molecules that enhance LNP uptake into cells. This has led to the identification of cardiac glycosides as potential general agents to enhance the uptake of LNP siRNA systems into cells as the Na⁺/K⁺-ATPase is expressed in all mammalian cells. Using strophanthidin as a representative cardiac glycoside, we have shown that LNP siRNA containing strophanthidin coupled to a PEG-lipid (STR-PEG-lipid) exhibit improved uptake and gene silencing properties in a variety of cell lines in vitro. Further, we have shown that LNP GAPDH siRNA systems containing STR-PEG-lipid exhibit potent gene silencing effects in kidney tissue in vivo following i.v. administration. This is the first time a systemically administered siRNA formulation has been shown to have gene silencing activity outside hepatocytes. The activity of LNP siRNA in liver has been attributed to the ability of LNP systems to accumulate Apo E following administration, which leads to LNP uptake by Apo E receptors on hepatocytes. The results presented here suggest that the therapeutic potential of LNP siRNA systems may be extended to other tissues such as the kidney by including small molecule ligands tethered to the LNP as agents to stimulate cell uptake. Optimization of the STR-PEG-lipid LNP siRNA system and gene silencing potencies in tissues other than liver and kidney are currently being investigated.

### Example 3

### More Targeted LNP are Taken Up by Cells

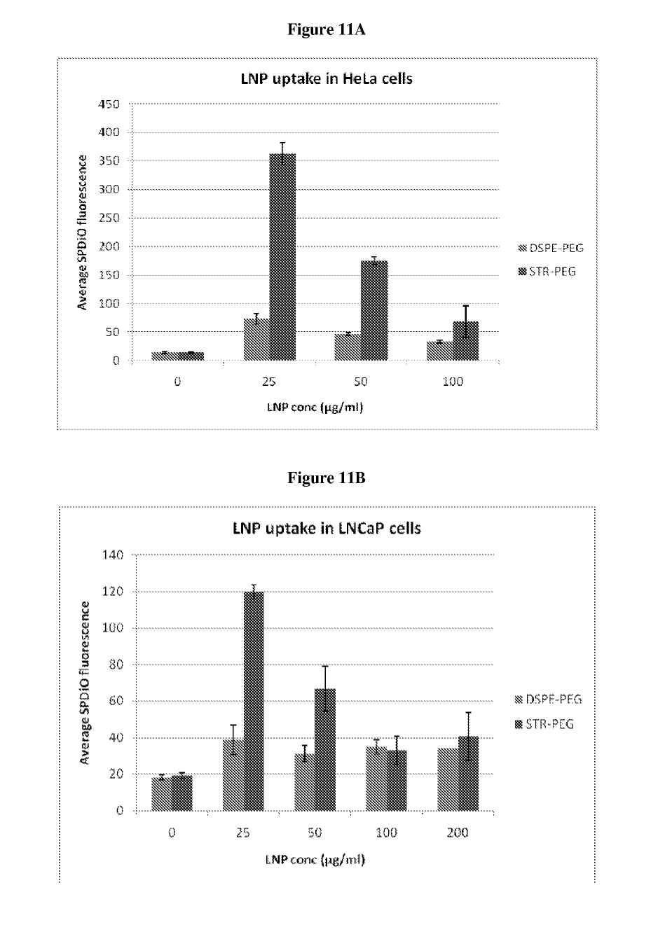

HeLa and LNCaP cells were incubated with targeted LNP containing strophanthidin-PEG (STR-PEG) or control LNP containing DSPE-PEG for 24 hrs at the indicated concentrations. LNP uptake was quantified by Cellomics as described in the prior Example. Representative images of HeLa cells are shown in FIG. 11A, and quantification of LNP uptake is shown in FIGS. 12B and 12C.

### Example 4

### Synthesis of 2,2-Dilinoleyl-4-dimethylaminomethyl-[1,3]-Dioxolane (DLin-K-DMA)

DLin-K-DMA was synthesized as shown in the following schematic and described below.

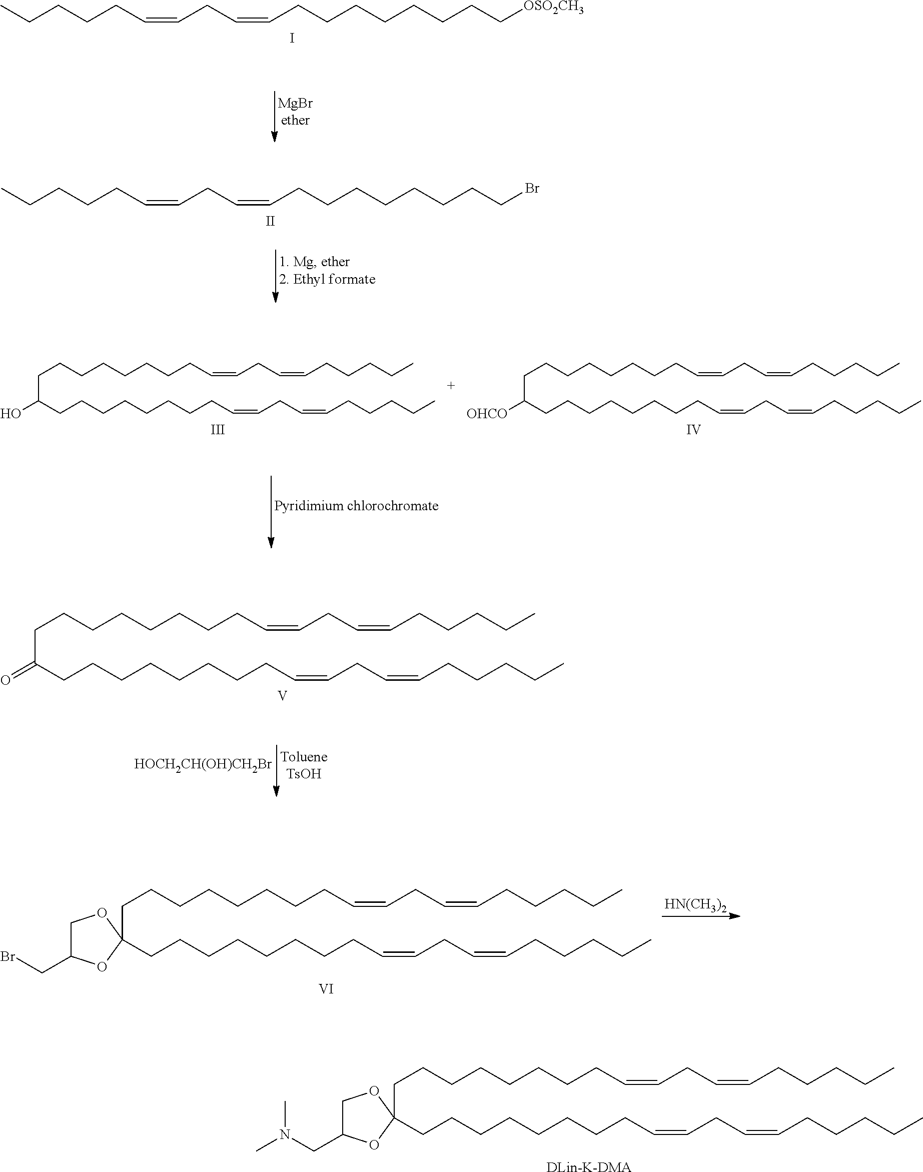

### Synthesis of Linoleyl Bromide (II)

A mixture of linoleyl methane sulfonate (6.2 g, 18 mmol) and magnesium bromide etherate (17 g, 55 mmol) in anhydrous ether (300 mL) was stirred under argon overnight (21 hours). The resulting suspension was poured into 300 mL of chilled water. Upon shaking, the organic phase was separated. The aqueous phase was extracted with ether (2×150 mL). The combined ether phase was washed with water (2×150 mL), brine (150 mL), and dried over anhydrous Na₂SO₄. The solvent was evaporated to afford 6.5 g of colourless oil. The crude product was purified by column chromatography on silica gel (230-400 mesh, 300 mL) eluted with hexanes. This gave 6.2 g (approximately 100%) of linoleyl bromide (II). ¹H NMR (400 MHz, CDCl₃) □: 5.27-5.45 (4H, m, 2×CH═CH), 3.42 (2H, t, CH₂Br), 2.79 (2H, t, C═C—CH₂—C═C), 2.06 (4H, q, 2×allylic CH₂), 1.87 (2H, quintet, CH₂), 1.2-1.5 (16H, m), 0.90 (3H, t, CH₃) ppm.

### Synthesis of Dilinoleyl Methanol (III)

To a suspension of Mg turnings (0.45 g, 18.7 mmol) with one crystal of iodine in 200 mL of anhydrous ether under nitrogen was added a solution of linoleyl bromide (II) in 50 mL of anhydrous ether at room temperature. The resulting mixture was refluxed under nitrogen overnight. The mixture was cooled to room temperature. To the cloudy mixture under nitrogen was added dropwise at room temperature a solution of ethyl formate (0.65 g, 18.7 mmol) in 30 mL of anhydrous ether. Upon addition, the mixture was stirred at room temperature overnight (20 hours). The ether layer was washed with 10% H₂SO₄ aqueous solution (100 mL), water (2×100 mL), brine (150 mL), and then dried over anhydrous Na₂SO₄. Evaporation of the solvent gave 5.0 g of pale oil. Column chromatography on silica gel (230-400 mesh, 300 mL) with 0-7% ether gradient in hexanes as eluent afforded two products, dilinoleyl methanol (2.0 g, III) and dilinoleylmethyl formate (1.4 g, IV). ¹H NMR (400 MHz, CDCl₃) for dilinoleylmethyl formate (IV) □: 8.10 (1H, s, CHO), 5.27-5.45 (8H, m, 4×CH═CH), 4.99 (1H, quintet, OCH), 2.78 (4H, t, 2×C═C—CH₂—C═C), 2.06 (8H, q, 4×allylic CH₂), 1.5-1.6 (4H, m, 2×CH₂), 1.2-1.5 (32H, m), 0.90 (6H, t, 2×CH₃) ppm.

Dilinoleylmethyl formate (IV, 1.4 g) and KOH (0.2 g) were stirred in 85% EtOH at room temperature under nitrogen overnight. Upon completion of the reaction, half of the solvent was evaporated. The resulting mixture was poured into 150 mL of 5% HCL solution. The aqueous phase was extracted with ether (3×100 mL). The combined ether extract was washed with water (2×100 mL), brine (100 mL), and dried over anhydrous Na2SO4. Evaporation of the solvent gave 1.0 g of dilinoleyl methanol (III) as colourless oil. Overall, 3.0 g (60%) of dilinoleyl methanol (III) were afforded. ¹H NMR (400 MHz, CDCl₃) for dilinoleyl methanol (III) δ: ppm.

### Synthesis of Dilinoleyl Ketone (V)

To a mixture of dilinoleyl methanol (2.0 g, 3.8 mmol) and anhydrous sodium carbonate (0.2 g) in 100 mL of CH₂Cl₂ was added pydimium chlorochromate (PCC, 2.0 g, 9.5 mmol). The resulting suspension was stirred at room temperature for 60 min. Ether (300 mL) was then added into the mixture, and the resulting brown suspension was filtered through a pad of silica gel (300 mL). The silica gel pad was further washed with ether (3×200 mL). The ether filtrate and washes were combined. Evaporation of the solvent gave 3.0 g of an oily residual as a crude product. The crude product was purified by column chromatography on silica gel (230-400 mesh, 250 mL) eluted with 0-3% ether in hexanes. This gave 1.8 g (90%) of dilinoleyl ketone (V). ¹H NMR (400 MHz, CDCl₃) δ: 5.25-5.45 (8H, m, 4×CH═CH), 2.78 (4H, t, 2×C═C—CH₂—C═C), 2.39 (4H, t, 2×COCH₂), 2.05 (8H, q, 4×allylic CH₂), 1.45-1.7 (4H, m), 1.2-1.45 (32H, m), 0.90 (6H, t, 2×CH₃) ppm.

### Synthesis of 2,2-Dilinoleyl-4-bromomethyl-[1,3]-dioxolane (VI)

A mixture of dilinoleyl methanol (V, 1.3 g, 2.5 mmol), 3-bromo-1,2-propanediol (1.5 g, 9.7 mmol) and p-toluene sulonic acid hydrate (0.16 g, 0.84 mmol) in 200 mL of toluene was refluxed under nitrogen for 3 days with a Dean-Stark tube to remove water. The resulting mixture was cooled to room temperature. The organic phase was washed with water (2×50 mL), brine (50 mL), and dried over anhydrous Na₂SO₄. Evaporation of the solvent resulted in a yellowish oily residue. Column chromatography on silica gel (230-400 mesh, 100 mL) with 0-6% ether gradient in hexanes as eluent afforded 0.1 g of pure VI and 1.3 g of a mixture of VI and the starting material. ¹H NMR (400 MHz, CDCl₃) δ: 5.27-5.45 (8H, m, 4×CH═CH), 4.28-4.38 (1H, m, OCH), 4.15 (1H, dd, OCH), 3.80 (1H, dd, OCH), 3.47 (1H, dd, CHBr), 3.30 (1H, dd, CHBr), 2.78 (4H, t, 2×C═C—CH₂—C═C), 2.06 (8H, q, 4×allylic CH₂), 1.52-1.68 (4H, m, 2×CH₂), 1.22-1.45 (32H, m), 0.86-0.94 (6H, m, 2×CH₃) ppm.

### Synthesis of 2,2-Dilinoleyl-4-dimethylaminomethyl-[1,3]-dioxolane (DLin-K-DMA)

Anhydrous dimethyl amine was bubbled into an anhydrous THF solution (100 mL) containing 1.3 g of a mixture of 2,2-dilinoleyl-4-bromomethyl-[1,3]-dioxolane (VI) and dilinoleyl ketone (V) at 0° C. for 10 min. The reaction flask was then sealed and the mixture stirred at room temperature for 6 days. Evaporation of the solvent left 1.5 g of a residual. The crude product was purified by column chromatography on silica gel (230-400 mesh, 100 mL) and eluted with 0-5% methanol gradient in dichloromethane. This gave 0.8 g of the desired product DLin-K-DMA. ¹H NMR (400 MHz, CDCl₃) δ: 5.25-5.45 (8, m, 4×CH═CH), 4.28-4.4 (1H, m, OCH), 4.1 (1H, dd, OCH), 3.53 (1H, t OCH), 2.78 (4H, t, 2×C═C—CH₂—C═C), 2.5-2.65 (2H, m, NCH₂), 2.41 (6H, s, 2×NCH₃), 2.06 (8H, q, 4×allylic CH₂), 1.56-1.68 (4H, m, 2×CH₂), 1.22-1.45 (32H, m), 0.90 (6H, t, 2×CH₃) ppm.

### Example 5

### Synthesis of 1,2-Dilinoleyloxy-N,N-dimethyl-3-aminopropane (DLinDMA) DLinDMA was Synthesized as Described Below

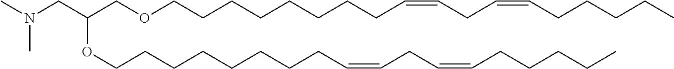

### 1,2-Dilinoleyloxy-3-dimethylaminopropane (DLinDMA)

To a suspension of NaH (95%, 5.2 g, 0.206 mol) in 120 mL of anhydrous benzene was added dropwise N,N-dimethyl-3-aminopropane-1,2-diol (2.8 g, 0.0235 mol) in 40 mL of anhydrous benzene under argon. Upon addition, the resulting mixture was stirred at room temperature for 15 min. Linoleyl methane sulfonate (99%, 20 g, 0.058 mol) in 75 mL of anhydrous benzene was added dropwise at room temperature under argon to the above mixture. After stirred at room temperature for 30 min., the mixture was refluxed overnight under argon. Upon cooling, the resulting suspension was treated dropwise with 250 mL of 1:1 (V:V) ethanol-benzene solution. The organic phase was washed with water (150 mL), brine (2×200 mL), and dried over anhydrous sodium sulfate. Solvent was evaporated in vacuo to afford 17.9 g of light oil as a crude product. 10.4 g of pure DLinDMA were obtained upon purification of the crude product by column chromatography twice on silica gel using 0-5% methanol gradient in methylene chloride. ¹H NMR (400 MHz, CDCl₃) δ: 5.35 (8H, m, CH═CH), 3.5 (7H, m, OCH), 2.75 (4H, t, 2×CH₂), 2.42 (2H, m, NCH₂), 2.28 (6H, s, 2×NCH₃), 2.05 (8H, q, vinyl CH₂), 1.56 (4H, m, 2×CH₂), 1.28 (32H, m, 16×CH₂), 0.88 (6H, t, 2×CH₃) ppm.

### Example 6

### Synthesis of 1,2-Dilinolenoyl-3-dimethylaminopropane

1,2-Dilinolenoyl-3-N,N-dimethylaminopropane (DLinDAP) was synthesized as described below.

To a solution of linoleic acid (99%, 49.7 g, 0.177 mol) in 800 mL of anhydrous benzene was added dropwise oxalyl chloride (99%, 29.8 g, 0.235 mol) under argon. Upon addition, the resulting mixture was stirred at room temperature for 2 hours until no bubble was released. The solvent and excess of oxalyl chloride was removed in vacuo. To the residual was added anhydrous benzene (1 L) followed by a solution of 3-N,N-dimethylamino-1,2-propanediol and dry pyridine in anhydrous benzene (100 mL) dropwise. The resulting mixture was stirred at room temperature for 2 days. Upon evaporation of the solvent, 64 g of yellowish syrup were afforded. 19 g of pure DLinDAP were obtained upon purification of the crude product by column chromatography three times on silica gel using 0-5% methanol gradient in chloroform. ¹H NMR (400 MHz, CDCl₃) δ: 5.49 (1H, m), 5.43-5.26 (8H, m), 4.41 (1H, dd), 4.13 (1H, dd), 3.15-3.35 (2H, m), 2.82 (6H, s, 2×NCH₃), 2.76 (4H, t), 2.35-2.6 (2H, m), 2.31 (2H, t), 2.03 (8H, q, vinyl CH₂), 1.53-1.68 (4H, m, 2×CH₂), 1.2-1.4 (28H, m, 14×CH₂), 0.88 (6H, t, 2×CH₃) ppm.

### Example 7

### Synthesis of 2,2-Dilinoleyl-4-(2-dimethylaminoethyl)-[1,3]-dioxolane (DLin-K-C2-DMA)

DLin-K-C2-DMA was synthesized as shown in the schematic diagram and description below.

### Synthesis of 2,2-Dilinoleyl-4-(2-hydroxyethyl)-[1,3]-dioxolane (II)

A mixture of dilinoleyl ketone (I, previously prepared as described in Example 1, 527 mg, 1.0 mmol), 1,3,4-butanetriol (technical grade, ca. 90%, 236 mg, 2 mmol) and pyridinium p-toluenesulfonate (50 mg, 0.2 mmol) in 50 mL of toluene was refluxed under nitrogen overnight with a Dean-Stark tube to remove water. The resulting mixture was cooled to room temperature. The organic phase was washed with water (2×30 mL), brine (50 mL), and dried over anhydrous Na₂SO₄. Evaporation of the solvent resulted in a yellowish oily residual (0.6 g). The crude product was purified by column chromatography on silica gel (230-400 mesh, 100 mL) with dichloromethane as eluent. This afforded 0.5 g of pure II as colourless oil. ¹H NMR (400 MHz, CDCl₃) δ: 5.25-5.48 (8H, m, 4×CH═CH), 4.18-4.22 (1H, m, OCH), 4.08 (1H, dd, OCH), 3.82 (2H, t, OCH₂), 3.53 (1H, t, OCH), 2.78 (4H, t, 2×C═C—CH₂—C═C), 2.06 (8H, q, 4×allylic CH₂), 1.77-1.93 (2H, m, CH₂), 1.52-1.68 (4H, m, 2×CH₂), 1.22-1.45 (32H, m), 0.86-0.94 (6H, t, 2×CH₃) ppm.

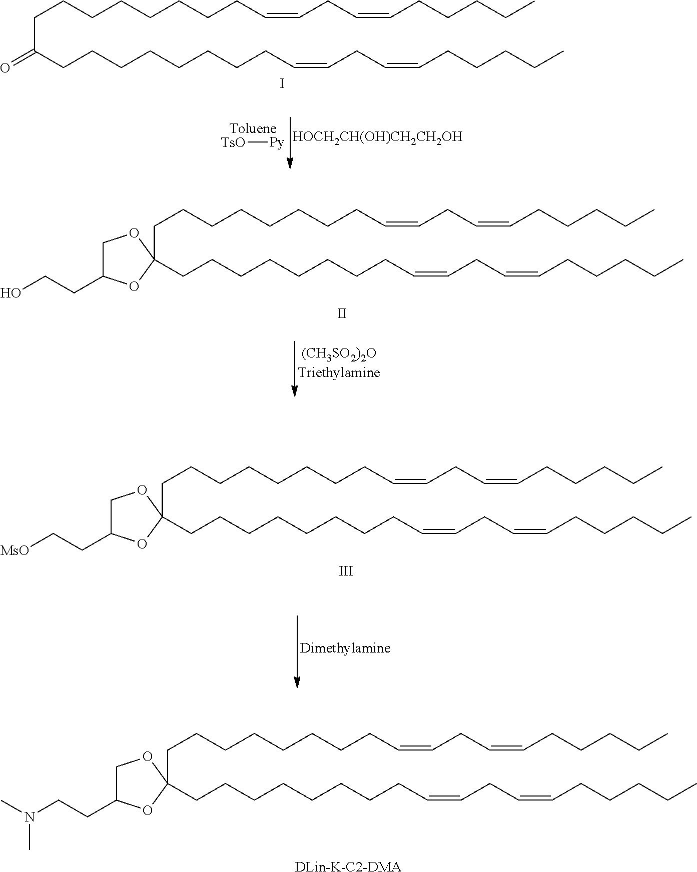

### Synthesis of 2,2-Dilinoleyl-4-(2-methanesulfonylethyl)-[1,3]-dioxolane (III)

To a solution of 2,2-dilinoleyl-4-(2-hydroxyethyl)-[1,3]-dioxolane (II, 500 mg, 0.81 mmol) and dry triethylamine (218 mg, 2.8 mmol) in 50 mL of anhydrous CH₂Cl₂ was added methanesulfonyl anhydride (290 mg, 1.6 mmol) under nitrogen. The resulting mixture was stirred at room temperature overnight. The mixture was diluted with 25 mL of CH₂Cl₂. The organic phase was washed with water (2×30 mL), brine (50 mL), and dried over anhydrous Na₂SO₄. The solvent was evaporated to afford 510 mg of yellowish oil. The crude product was used in the following step without further purification.

### Synthesis of 2,2-Dilinoleyl-4-(2-dimethylaminoethyl)-[1,3]-dioxolane (DLin-K-C2-DMA)

To the above crude material (III) under nitrogen was added 20 mL of dimethylamine in THF (2.0 M). The resulting mixture was stirred at room temperature for 6 days. An oily residual was obtained upon evaporation of the solvent. Column chromatography on silica gel (230-400 mesh, 100 mL) with 0-5% methanol gradient in dichloromethane as eluent resulted in 380 mg of the product DLin-K-C2-DMA as pale oil. ¹H NMR (400 MHz, CDCl₃) δ: 5.27-5.49 (8, m, 4×CH═CH), 4.01-4.15 (2H, m, 2×OCH), 3.49 (1H, t OCH), 2.78 (4H, t, 2×C═C—CH₂—C═C), 2.34-2.54 (2H, m, NCH₂), 2.30 (6H, s, 2×NCH₃), 2.06 (8H, q, 4×allylic CH₂), 1.67-1.95 (2H, m, CH₂), 1.54-1.65 (4H, m, 2×CH₂), 1.22-1.45 (32H, m), 0.90 (6H, t, 2×CH₃) ppm.

### Example 8

### Synthesis of 2,2-Dilinoleyl-4,5-bis(dimethylamino methyl)-[1,3]-dioxolane (DLin-K-DMA)

DLin-K-DMA was synthesized as described and shown in the schematic diagrams below.

### Synthesis of D-Lin-K-diethyltartarate (II)

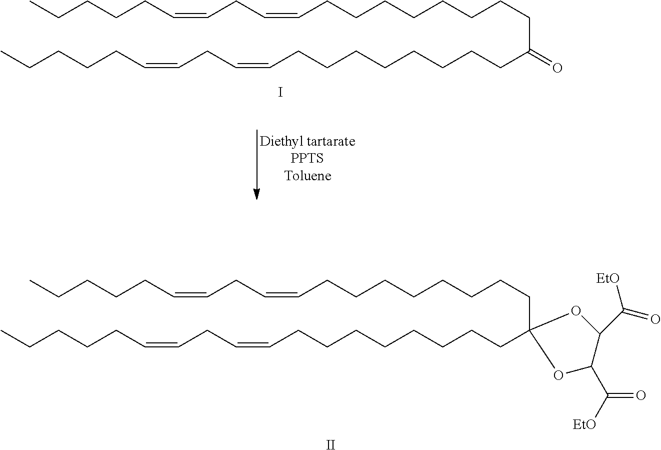

A mixture of D-Lin-Ketone (I, 1 gram, 1.9 mmol), Diethyl-D-tartarate (412 mg, 2 mmol) and Pyridinium p-tolene sulfonate (250 mg, 1 mmol) in 25 mL of toluene was refluxed under nitrogen for two days with a Dean-stark tube to remove water. The resulting mixture was cooled to room temperature. The organic phase was washed with water NaHCO₃ and brine (2×50 mL) and dried over anhydrous Na₂SO₄. Evaporation of the solvent resulted in yellowish oily residue. Column chromatography on silica gel (230-400 mesh, 500 mL) eluted with 0-10% ether gradients in hexanes as eluent afforded 400 mg of pure D-Lin-diethyltartarate (II).

¹H NMR (300 MHz, CDCl₃) δ: 5.27-5.46 (8H, m), 4.67 (2H, s), 4.20-4.30 (1H, t), 2.75 (4H, t), 2.02-2.09 (8H, m) 1.62-1.72 (4H, m), 1.2-1.47 (32H, m), 0.87-0.90 (6H, t) ppm.

### Synthesis of D-Lin-K-diethyldiol (III)

To a solution of Lithiumaluminiumhydride (32 mg, 1 mmol) in dry THF a solution of D-Lin-K-diethyltartarate (II, 600 mg, 0.85 m mol) was added in dry THF at 0° C. under argon atmosphere and then the reaction was stirred for four hours at room temperature. The reaction mixture was quenched with ice cold water and then filtered through celite and the evaporation of solvent gave crude reduced alcohol. Column chromatography on silica gel (230-400 mesh, 500 mL) eluted with 10-40% ethyl acetate gradients in hexanes as eluent afforded 350 mg of pure D-Lin-diethyltartarate (III).

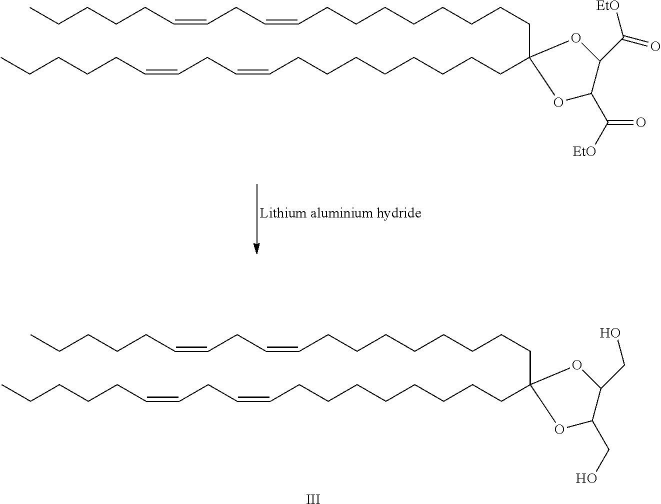

¹H NMR (300 MHz, CDCl₃) δ: 5.27-5.46 (8H, m), 3.95 (2H, t), 3.65-3.85 (4H, dd), 2.75 (4H, t), 2.02-2.09 (8H, m) 1.62-1.72 (4H, m), 1.2-1.47 (32H, m), 0.87-0.90 (6H, t) ppm.

### Synthesis of D-Lin-K-diethyldimesylate (IV)

To a mixture of D-Lin-K-diethyltartarate (III) alcohol (570 mg, 0.95 mmol) in dry dichloromethane pyridine (275 mg, 3.85 mmol) and 4-(Dimethylamino)pyridine (122 mg, 1 mmol) was added under argon atmosphere to this solution a solution of methane sulfonyl chloride (500 mg, 2.5 mmol) was slowly added and stirred over night.

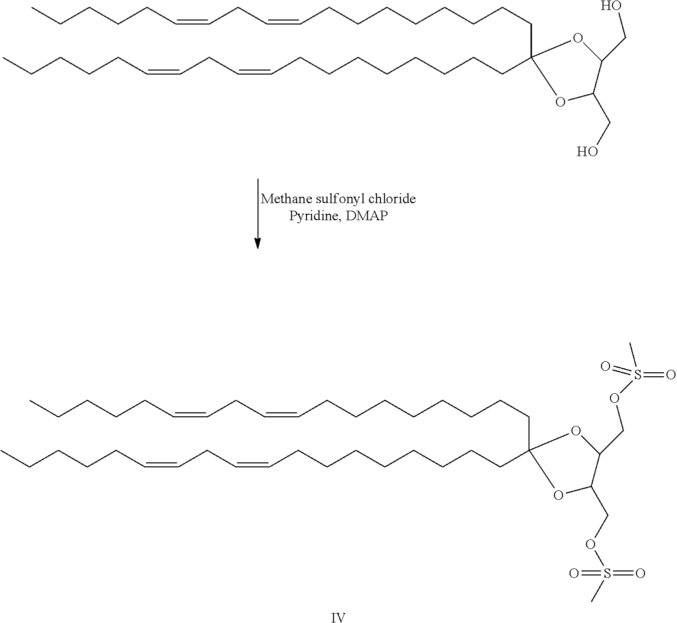

The organic phase was washed with water and brine (2×50 mL) then solvent was evaporated to give yellowish oil residue. Purified over Column chromatography on silica gel (230-400 mesh, 500 mL), eluted with 10-40% ethyl acetate gradients in hexanes as eluent, afforded 300 mg of pure D-Lin-diethyltartarate (IV).

¹H NMR (300 MHz, CDCl₃) δ: 5.27-5.46 (8H, m), 4.35 (4H, d), 4.12-4.17 (2H, t), 3.08 (6H, s), 2.75 (4H, t), 2.02-2.09 (8H, m) 1.62-1.72 (4H, m), 1.2-1.47 (32H, m), 0.87-0.90 (6H, t) ppm.

### Synthesis of D-Lin-K²-DMA

Anhydrous dimethyl amine solution in THF was added to the reaction vessel containing (300 mg) of D-Lin-diethyltartarate (IV) at room temperature for 5 min. the reaction flask was then sealed and the mixture stirred at room temperature for 6 days. Evaporation of the solvent left 300 mg of residual. The crude product was purified by column chromatography on silica gel (230-400 mesh, 500 mL) eluted with 0-10% Methanol gradients in chloroform as eluent afforded 50 mg of pure D-Lin-K²-DMA.

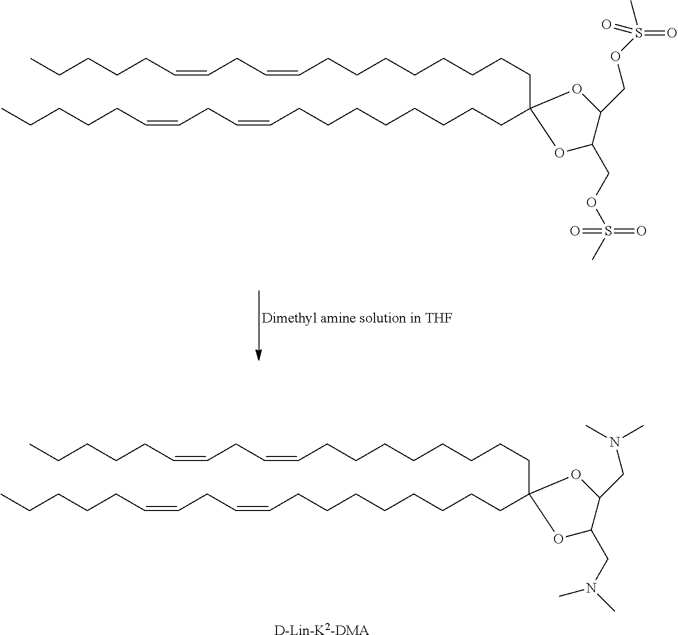

¹H NMR (300 MHz, CDCl₃) δ: 5.27-5.46 (8H, m), 3.72-3.80 (2H, t), 2.75 (4H, t), 2.49 (4H, d), 2.30 (12H, s), 2.02-2.09 (8H, m) 1.62-1.72 (4H, m), 1.2-1.47 (32H, m), 0.87-0.90 (6H, t) ppm.

### Example 9

### Synthesis of Targeted Lipid

The synthesis of a targeted lipid, strophanthidin-PEG (STR-PEG) is depicted in the schematic diagram below.

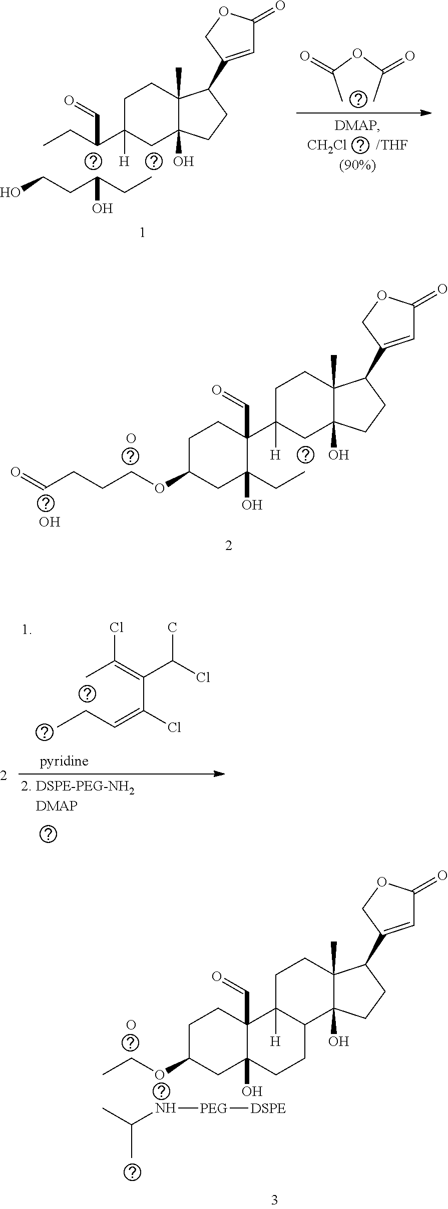

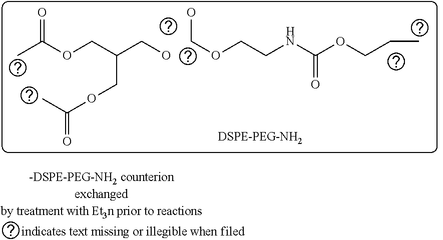

A handle for the conjugation of strophanthidin (1) to a readily available PEG-functionalized phospholipid (DSPE-PEG-NH₂) was installed treating cardenolide 1 with succinic anhydride in the presence of 4-dimethylaminopyridine (DMAP) at room temperature to furnish carboxylic acid 2 in high yield. Where conventional peptide coupling methods failed, exposure of succinate 2 to Yamaguchi's reagent in pyridine furnished the mixed anhydride, which directly treated with DSPE-PEG-NH₂ and DMAP, giving lipid conjugate 3 after careful chromatography on silica gel.

### Example 10

### Synthesis of mPEG2000-1,2-Di-O-Alkyl-sn3-Carbomoylglyceride (PEG-C-DOMG)

Certain PEG-lipids, such as mPEG2000-1,2-Di-O-Alkyl-sn3-Carbomoylglyceride (PEG-C-DOMG) are synthesized as shown in the schematic and described below.

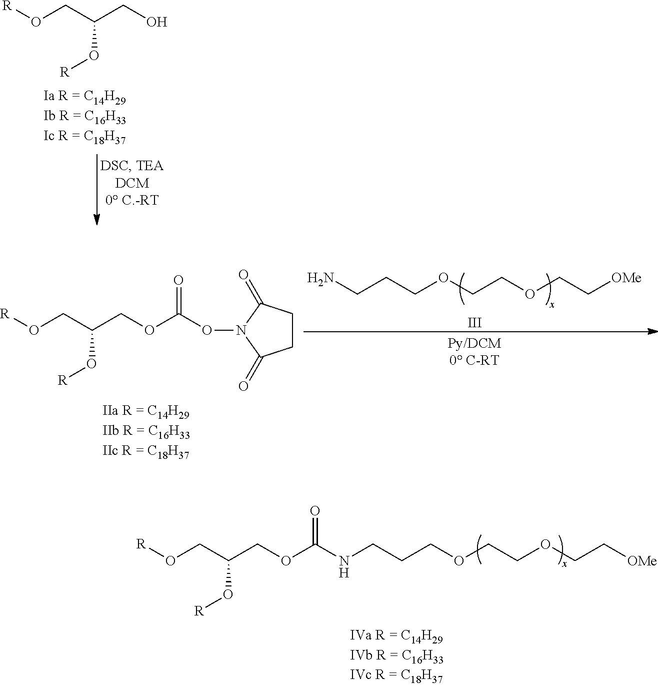

### Synthesis of IVa

1,2-Di-O-tetradecyl-sn-glyceride Ia (30 g, 61.80 mmol) and N,N′-succinimidylcarboante (DSC, 23.76 g, 1.5 eq) were taken in dichloromethane (DCM, 500 mL) and stirred over an ice water mixture. Triethylamine (TEA, 25.30 mL, 3 eq) was added to the stirring solution and subsequently the reaction mixture was allowed to stir overnight at ambient temperature. Progress of the reaction was monitored by TLC. The reaction mixture was diluted with DCM (400 mL) and the organic layer was washed with water (2×500 mL), aqueous NaHCO₃ solution (500 mL) followed by standard work-up. The residue obtained was dried at ambient temperature under high vacuum overnight. After drying, the crude carbonate IIa thus obtained was dissolved in dichloromethane (500 mL) and stirred over an ice bath. To the stirring solution, mPEG₂₀₀₀-NH₂ (III, 103.00 g, 47.20 mmol, purchased from NOF Corporation, Japan) and anhydrous pyridine (Py, 80 mL, excess) were added under argon. In some embodiments, the x in compound III has a value of 45-49, preferably 47-49, and more preferably 49. The reaction mixture was then allowed to stir at ambient temperature overnight. Solvents and volatiles were removed under vacuum and the residue was dissolved in DCM (200 mL) and charged on a column of silica gel packed in ethyl acetate. The column was initially eluted with ethyl acetate and subsequently with gradient of 5-10% methanol in dichloromethane to afford the desired PEG-Lipid IVa as a white solid (105.30 g, 83%). ¹H NMR (CDCl₃, 400 MHz) δ=5.20-5.12 (m, 1H), 4.18-4.01 (m, 2H), 3.80-3.70 (m, 2H), 3.70-3.20 (m, —O—CH₂—CH₂—O—, PEG-CH₂), 2.10-2.01 (m, 2H), 1.70-1.60 (m, 2H), 1.56-1.45 (m, 4H), 1.31-1.15 (m, 48H), 0.84 (t, J=6.5 Hz, 6H). MS range found: 2660-2836.

### Synthesis of IVb

1,2-Di-O-hexadecyl-sn-glyceride Ib (1.00 g, 1.848 mmol) and DSC (0.710 g, 1.5 eq) were taken together in dichloromethane (20 mL) and cooled down to 0° C. in an ice water mixture. Triethylamine (1.00 mL, 3 eq) was added and the reaction was stirred overnight. The reaction was followed by TLC, diluted with DCM, washed with water (2 times), NaHCO₃ solution and dried over sodium sulfate. Solvents were removed under reduced pressure and the resulting residue of IIb was maintained under high vacuum overnight. This compound was directly used for the next reaction without further purification. MPEG₂₀₀₀-NH₂ III (1.50 g, 0.687 mmol, purchased from NOF Corporation, Japan) and IIb (0.702 g, 1.5 eq) were dissolved in dichloromethane (20 mL) under argon. In some embodiments, the x in compound III has a value of 45-49, preferably 47-49, and more preferably 49. The reaction was cooled to 0° C. Pyridine (1 mL, excess) was added and the reaction stirred overnight. The reaction was monitored by TLC. Solvents and volatiles were removed under vacuum and the residue was purified by chromatography (first ethyl acetate followed by 5-10% MeOH/DCM as a gradient elution) to obtain the required compound IVb as a white solid (1.46 g, 76%). ¹H NMR (CDCl₃, 400 MHz) δ=5.17 (t, J=5.5 Hz, 1H), 4.13 (dd, J=4.00 Hz, 11.00 Hz, 1H), 4.05 (dd, J=5.00 Hz, 11.00 Hz, 1H), 3.82-3.75 (m, 2H), 3.70-3.20 (m, —O—CH₂—CH₂—O—, PEG-CH₂), 2.05-1.90 (m, 2H), 1.80-1.70 (m, 2H), 1.61-1.45 (m, 6H), 1.35-1.17 (m, 56H), 0.85 (t, J=6.5 Hz, 6H). MS range found: 2716-2892.

### Synthesis of IVc

1,2-Di-O-octadecyl-sn-glyceride Ic (4.00 g, 6.70 mmol) and DSC (2.58 g, 1.5 eq) were taken together in dichloromethane (60 mL) and cooled down to 0° C. in an ice water mixture. Triethylamine (2.75 mL, 3 eq) was added and the reaction was stirred overnight. The reaction was followed by TLC, diluted with DCM, washed with water (2 times), NaHCO₃ solution, and dried over sodium sulfate. Solvents were removed under reduced pressure and the residue was maintained under high vacuum overnight. This compound was directly used for the next reaction without further purification. MPEG₂₀₀₀-NH₂ III (1.50 g, 0.687 mmol, purchased from NOF Corporation, Japan) and IIc (0.760 g, 1.5 eq) were dissolved in dichloromethane (20 mL) under argon. In some embodiments, the x in compound III has a value of 45-49, preferably 47-49, and more preferably 49. The reaction was cooled to 0° C. Pyridine (1 mL, excess) was added and the reaction was stirred overnight. The reaction was monitored by TLC. Solvents and volatiles were removed under vacuum and the residue was purified by chromatography (ethyl acetate followed by 5-10% MeOH/DCM as a gradient elution) to obtain the desired compound IVc as a white solid (0.92 g, 48%). ¹H NMR (CDCl₃, 400 MHz) δ=5.22-5.15 (m, 1H), 4.16 (dd, J=4.00 Hz, 11.00 Hz, 1H), 4.06 (dd, J=5.00 Hz, 11.00 Hz, 1H), 3.81-3.75 (m, 2H), 3.70-3.20 (m, —O—CH₂—CH₂—O—, PEG-CH₂), 1.80-1.70 (m, 2H), 1.60-1.48 (m, 4H), 1.31-1.15 (m, 64H), 0.85 (t, J=6.5 Hz, 6H). MS range found: 2774-2948.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Aizman, O., and A. Aperia. 2003. Na,K-ATPase as a signal transducer. Ann N Y Acad Sci. 986:489-96.
Albengres, E., Urien, S., Barre, J., Nguyen, P., Bree, F., Jolliet, P., Tillement, J. P., Tsai, R. S., Carrupt, P. A. and Testa, B. (1993). Clinical pharmacology of oxicams: new insights into the mechanisms of their dose-dependent toxicity. Int J Tissue React. 15(3), 125-34.
Akinc, A., Zumbuehl, A., Goldberg, M., Leshchiner, E. S., Busini, V., Hossain, N., Bacallado, S. A., Nguyen, D. N., Fuller, J., Alvarez, R., Borodovsky, A., Borland, T., Constien, R., de Fougerolles, A., Dorkin, J. R., Narayanannair Jayaprakash, K., Jayaraman, M., John, M., Koteliansky, V., Manoharan, M., Nechev, L., Qin, J., Racie, T., Raitcheva, D., Rajeev, K. G., Sah, D. W., Soutschek, J., Toudjarska, I., Vornlocher, H. P., Zimmermann, T. S., Langer, R. and Anderson, D. G. (2008). A combinatorial library of lipid-like materials for delivery of RNAi therapeutics. Nat. Biotechnol. 26(5), 561-9.
Antonipillai, I., K. Schick, and R. Horton. 1996. Ouabain is a potent inhibitor of aldosterone secretion and angiotensin action in the human adrenal. J Clin Endocrinol Metab. 81:2335-7.
Auguste, D. T., Furman, K., Wong, A., Fuller, J., Armes, S. P., Deming, T. J. and Langer, R. (2008). Triggered release of siRNA from poly(ethylene glycol)-protected, pH-dependent liposomes. J Control Release. 130(3), 266-74.
Banerjee, R., P. Tyagi, S. Li, and L. Huang. 2004. Anisamide-targeted stealth liposomes: a potent carrier for targeting doxorubicin to human prostate cancer cells. Int J Cancer. 112:693-700.
Barber, R. D., Harmer, D. W., Coleman, R. A. and Clark, B. J. (2005). GAPDH as a housekeeping gene: analysis of GAPDH mRNA expression in a panel of 72 human tissues. Physiol Genomics. 21(3), 389-95.
Basha, G., Rosin, N., Tam, Y. Y. C., Hafez, I. M., Wong, M. and Cullis, P. R. (2010). Influence of cationic lipid composition on uptake, intracellular trafficking and gene silencing properties of siRNA in primary antigen presenting cells. (In preparation).
Budker, V., Gurevich, V., Hagstrom, J. E., Bortzov, F. and Wolff, J. A. (1996). pH-sensitive, cationic liposomes: a new synthetic virus-like vector. Nat. Biotechnol. 14(6), 760-4.
Chapman, R. E. and Munro, S. (1994). Retrieval of TGN proteins from the cell surface requires endosomal acidification. EMBO J. 13(10), 2305-12.
Connor, J. and Huang, L. (1986). pH-sensitive immunoliposomes as an efficient and target-specific carrier for antitumor drugs. Cancer Res. 46(7), 3431-5.
Cushley, M. J. and Holgate, S. T. (1985). Bronchodilator actions of xanthine derivatives administered by inhalation in asthma. Thorax. 40(3), 176-9.
de Fougerolles, A., H. P. Vornlocher, J. Maraganore, and J. Lieberman. 2007. Interfering with disease: a progress report on siRNA-based therapeutics. Nat Rev Drug Discov. 6:443-53.
Dorsett, Y., and T. Tuschl. 2004. siRNAs: applications in functional genomics and potential as therapeutics. Nat Rev Drug Discov. 3:318-29.
Erbacher, P., Roche, A. C., Monsigny, M. and Midoux, P. (1996). Putative role of chloroquine in gene transfer into a human hepatoma cell line by DNA/lactosylated polylysine complexes. Exp Cell Res. 225(1), 186-94.
Farhood, H., Serbina, N. and Huang, L. (1995). The role of dioleoyl phosphatidylethanolamine in cationic liposome mediated gene transfer. Biochim Biophys Acta. 1235(2), 289-95.
Fenske, D. B., A. Chonn, and P. R. Cullis. 2008. Liposomal Nanomedicines: An Emerging Field. Toxicol Pathol. 36:21-29.
Finkbeiner, A. E., Bissada, N. K. and Welch, L. T. (1977). Uropharmacology: part VI. Parasympathetic depressants. Urology. 10(5), 503-10.
Garg, A., Tisdale, A. W., Haidari, E. and Kokkoli, E. (2009) Targeting colon cancer cells using PEGylated liposomes modified with a fibronectin-mimetic peptide. Int J Pharm. 366(1-2), 201-10.
Guy, J., Drabek, D. and Antoniou, M. (1995). Delivery of DNA into mammalian cells by receptor-mediated endocytosis and gene therapy. Mol Biotechnol. 3(3), 237-48.
Hasan, M. T., Subbaroyan, R. and Chang, T. Y. (1991). High-efficiency stable gene transfection using chloroquine-treated Chinese hamster ovary cells. Somat Cell Mol Genet. 17(5), 513-7.
Heyes, J., Palmer, L., Bremner, K. and MacLachlan, I. (2005). Cationic lipid saturation influences intracellular delivery of encapsulated nucleic acids. J Control Release. 107(2), 276-87.
Jeffs, L. B., L. R. Palmer, E. G. Ambegia, C. Giesbrecht, S. Ewanick, and I. MacLachlan. 2005. A scalable, extrusion-free method for efficient liposomal encapsulation of plasmid DNA. Pharm Res. 22:362-72.
Jolliet, P., Simon, N., Brée, F., Urien, S., Pagliara, A., Carrupt, P. A., Testa, B. and Tillement, J. P. (1997). Blood-to-brain transfer of various oxicams: effects of plasma binding on their brain delivery. Pharm Res. 14(5), 650-6.
Kakimoto, S., Hamada, T., Komatsu, Y., Takagi, M., Tanabe, T., Azuma, H., Shinkai, S, and Nagasaki, T. (2009). The conjugation of diphtheria toxin T domain to poly(ethylenimine) based vectors for enhanced endosomal escape during gene transfection. Biomaterials. 30(3), 402-8.
Kinoshita, M. and Hynynen, K. (2005). A novel method for the intracellular delivery of siRNA using microbubbe-enhanced focused ultrasound. Biochem Biophys Res Commun. 335(2), 393-9
Kolodny, A. L. (1960). A controlled study of trimethobenzamide (Tigan), a specific antiemetic. Am J Med Sci. 239, 682-9.
Kometiani, P., L. Liu, and A. Askari. 2005. Digitalis-induced signaling by Na+/K+-ATPase in human breast cancer cells. Mol Pharmacol. 67:929-36.
Kooi, C., Cervin, M. and Anderson, R. (1991). Differentiation of acid-pH-dependent and -nondependent entry pathways for mouse hepatitis virus. Virology. 180(1), 108-19.
Korzycka, L. and Górska, D. (2008). Synthesis, pharmacological activity and nitric oxide generation by nitrate derivatives of theophylline. J Pharm Pharmacol. 60(5), 637-45.
Kunisawa, J., Masuda, T., Katayama, K., Yoshikawa, T., Tsutsumi, Y., Akashi, M., Mayumi, T. and Nakagawa, S. (2005). Fusogenic liposome delivers encapsulated nanoparticles for cytosolic controlled gene release. J Control Release. 105(3), 344-53.
Li, S. D., and L. Huang. 2006. Targeted delivery of antisense oligodeoxynucleotide and small interference RNA into lung cancer cells. Mol Pharm. 3:579-88.
Lin, P. J. C., Tam, Y. Y. C., Hafez, I., Sandhu, A., Chen, S., Nabi. R. and Cullis, P. R. Influence of cationic lipid composition on uptake and intracellular processing of lipid nanoparticle formulation of siRNA in a macrophage cell line. (In preparation).
Liu, J., R. Kesiry, S. M. Periyasamy, D. Malhotra, Z. Xie, and J. I. Shapiro. 2004. Ouabain induces endocytosis of plasmalemmal Na/K-ATPase in LLC-PK1 cells by a clathrin-dependent mechanism. Kidney Int. 66:227-41.
Liu, J., M. Liang, L. Liu, D. Malhotra, Z. Xie, and J. I. Shapiro. 2005. Ouabain-induced endocytosis of the plasmalemmal Na/K-ATPase in LLC-PK1 cells requires caveolin-1. Kidney Int. 67:1844-54.
Magnussen, H., Jörres, R. and Hartmann, V. (1986). Bronchodilator effect of theophylline preparations and aerosol fenoterol in stable asthma. Chest. 90(5), 722-5.
Maurer, N., K. F. Wong, H. Stark, L. Louie, D. McIntosh, T. Wong, P. Scherrer, S. C. Semple, and P. R. Cullis. 2001. Spontaneous entrapment of polynucleotides upon electrostatic interaction with ethanol-destabilized cationic liposomes. Biophys J. 80:2310-26.
Negishi, Y., Endo, Y., Fukuyama, T., Suzuki, R., Takizawa, T., Omata, D., Maruyama, K. and Aramaki, Y. (2008). Delivery of siRNA into the cytoplasm by liposomal bubbles and ultrasound. J Control Release. 132(2), 124-30
Olkkola, K. T., Brunetto, A. V. and Mattila, M. J. (1994). Pharmacokinetics of oxicam nonsteroidal anti-inflammatory agents. Clin Pharmacokinet. 26(2), 107-20.
Paula, S., M. R. Tabet, and W. J. Ball, Jr. 2005. Interactions between cardiac glycosides and sodium/potassium-ATPase: three-dimensional structure-activity relationship models for ligand binding to the E2-Pi form of the enzyme versus activity inhibition. Biochemistry. 44:498-510.
Pless, D. D. and Wellner, R. B. (1996). In vitro fusion of endocytic vesicles: effects of reagents that alter endosomal pH. J Cell Biochem. 62(1), 27-39.
Reynolds, A., Leake, D., Boese, Q., Scaringe, S., Marshall, W. S. and Khvorova, A. (2004). Rational siRNA design for RNA interference. Nat Biotechnol. 22(3), 326-30.
Sapra, P., and T. M. Allen. 2003. Ligand-targeted liposomal anticancer drugs. Prog Lipid Res. 42:439-62.
Schoner, W., and G. Scheiner-Bobis. 2007. Endogenous and exogenous cardiac glycosides: their roles in hypertension, salt metabolism, and cell growth. Am J Physiol Cell Physiol. 293:C509-36.
Simons, F. E., Bierman, C. W., Sprenkle, A. C. and Simons, K. J. (1975). Efficacy of dyphylline (dihydroxypropyltheophylline) in exercise-induced bronchospasm. Pediatrics. 56(5 pt-2 suppl), 916-8.
Su, A. I., T. Wiltshire, S. Batalov, H. Lapp, K. A. Ching, D. Block, J. Zhang, R. Soden, M. Hayakawa, G. Kreiman, M. P. Cooke, J. R. Walker, and J. B. Hogenesch. 2004. A gene atlas of the mouse and human protein-encoding transcriptomes. Proc Natl Acad Sci USA. 101:6062-7.
Sweadner, K. J. 1989. Isozymes of the Na+/K+-ATPase. Biochim Biophys Acta. 988:185-220.
Tsiang, H. and Superti, F. (1984). Ammonium chloride and chloroquine inhibit rabies virus infection in neuroblastoma cells. Brief report. Arch Virol. 81(3-4), 377-82.
Watson, P., A. T. Jones, and D. J. Stephens. 2005. Intracellular trafficking pathways and drug delivery: fluorescence imaging of living and fixed cells. Adv Drug Deliv Rev. 57:43-61.
Xie, Z., and A. Askari. 2002. Na(+)/K(+)-ATPase as a signal transducer. Eur J. Biochem. 269:2434-9.
Young, M. D., and Eyles, D. E. (1948). The efficacy of chloroquine, quinacrine, quinine and totaquine in the treatment of Plasmodium malariae infections (quartan malaria). Am J Trop Med Hyg. 28(1), 23-8.
Zeichhardt, H., Wetz, K., Willingmann, P. and Habermehl, K. O. (1985). Entry of poliovirus type 1 and Mouse Elberfeld (ME) virus into HEp-2 cells: receptor-mediated endocytosis and endosomal or lysosomal uncoating. J Gen Virol. 66(Pt 3), 483-92.
Zelphati, O., and F. C. Szoka, Jr. 1996. Intracellular distribution and mechanism of delivery of oligonucleotides mediated by cationic lipids. Pharm Res. 13:1367-72.
Zhang, S., Zhao, B., Jiang, H., Wang, B. and Ma, B. (2007). Cationic lipids and polymers mediated vectors for delivery of siRNA. J Control Release. 123(1), 1-10.
Zimmermann, T. S., A. C. Lee, A. Akinc, B. Bramlage, D. Bumcrot, M. N. Fedoruk, J. Harborth, J. A. Heyes, L. B. Jeffs, M. John, A. D. Judge, K. Lam, K. McClintock, L. V. Nechev, L. R. Palmer, T. Racie, I. Rohl, S. Seiffert, S. Shanmugam, V. Sood, J. Soutschek, I. Toudjarska, A. J. Wheat, E. Yaworski, W. Zedalis, V. Koteliansky, M. Manoharan, H. P. Vornlocher, and I. MacLachlan. 2006. RNAi-mediated gene silencing in non-human primates. Nature. 441:111-4.
Zinnes, H., Sircar, J. C., Lindo, N., Schwartz, M. L., Fabian, A. C., Shavel, J. Jr., Kasulanis, C. F., Genzer, J. D., Lutomski, C. and DiPasquale, G. (1982). Isoxicam and related 4-hydroxy-N-isoxazolyl-2H-1,2-benzothiazine-3-carboxamide 1,1-dioxides. Potent nonsteroidal antiinflammatory agents. J Med Chem. 25(1), 12-8.

1. A conjugated lipid having the formula: 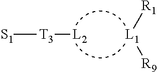
wherein
S₁ includes a quinoline moiety or a moiety that binds to Na+/K+-ATPase;
R¹ is a C₁₀ to C₃₀ group having the formula
-L^{1a}-(CR^{1a}R^{1b})_{α}-[L^{1b}-(CR^{1a}R^{1b})_{β}]_{γ}-L^{1c}-R^{1c},
wherein
L^{1a} is a bond, —CR^{1a}R^{1b}—, —O—, —CO—, —NR^{1d}—, —S—, or a combination thereof;
each R^{1a} and each R^{1b}, independently, is H; halo; hydroxy; cyano; C₁-C₆ alkyl optionally substituted by halo, hydroxy, or alkoxy; C₃-C₈cycloalkyl optionally substituted by halo, hydroxy, or alkoxy; —OR^{1c}; —NR^{1c}R^{1d}; aryl; heteroaryl; or heterocyclyl;
each L^{1b}, independently, is a bond, —(CR^{1a}R^{1b})₁₋₂—, —O—, —CO—, —NR^{1d}—, —S—, 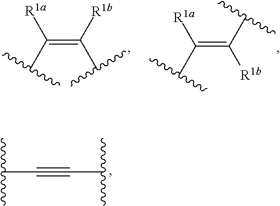
or a combination thereof; or has the formula 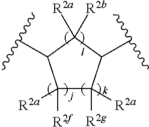
wherein j, k, and 1 are each independently 0, 1, 2, or 3, provided that the sum of j, k and 1 is at least 1 and no greater than 8; and R^{1f} and R^{1g} are each independently R^{1b}, or adjacent R^{1f} and R^{1g}, taken together, are optionally a bond;
or has the formula 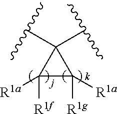
wherein j and k are each independently 0, 1, 2, 3, or 4 provided that the sum of j and k is at least 1; and R^{1f} and R^{1g} are each independently R^{1b}, or adjacent R^{1f} and R^{1g}, taken together, are optionally a bond;
or has the formula: 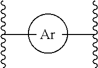
wherein —Ar— is a 6 to 14 membered arylene group optionally substituted by zero to six R^{1a} groups;
or has the formula: 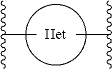
wherein -Het- is a 3 to 14 membered heterocyclylene or heteroarylene group optionally substituted by zero to six R^{1a} groups;
L^{1c} is —(CR^{1a}R^{1b})₁₋₂—, —O—, —CO—, —NR^{1d}—, —S—, 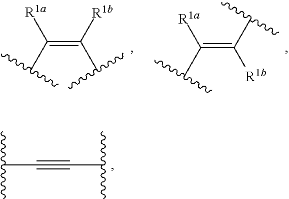
or a combination thereof;
R^{1c} is H; halo; hydroxy; cyano; C₁-C₆ alkyl optionally substituted by halo, hydroxy, or alkoxy; C₃-C₈ cycloalkyl optionally substituted by halo, hydroxy, or alkoxy; aryl; heteroaryl; or heterocyclyl; or R^{1c} has the formula: 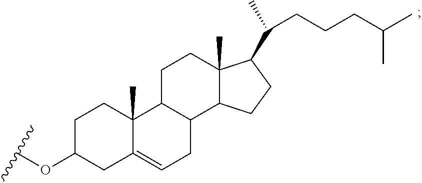
R^{1d} is H; halo; hydroxy; cyano; C₁-C₆ alkyl optionally substituted by halo, hydroxy, or alkoxy; C₃-C₈ cycloalkyl optionally substituted by halo, hydroxy, or alkoxy; aryl; heteroaryl; or heterocyclyl;
α is 0-6;
each β, independently, is 0-6; and
γ is 0-6;
R⁹ is R¹ or R⁵; 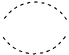
represents a connection between L₂ and L₁ which is:
(1) a single bond between one atom of L₂ and one atom of L₁, wherein
L₁ is C(Rₐ), O, S or N(Q);
L₂ is —(CR₅R₆)ₓ—, —C(O)—(CR₅R₆)ₓ—, —(CR₅R₆)ₓ—CR₅═CR₅—(CR₅R₆)_{y}—, —C(O)—(CR₅R₆)—CR₅═CR₅—(CR₅R₆)_{y}—, —O—, —S—, —N(Q)-, ═N—, ═C(R₅)—, —CR₅R₆—O—, —CR₅R₆—N(Q)-, —CR₅R₆—S—, —C(O)N(Q)-, —C(O)O—, —N(Q)C(O)—, —OC(O)—, —C(O)—, or —X—C(R₅)(YR₃)—;
wherein X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
Rₐ is H, alkyl, alkoxy, —OH, —N(Q)Q, or —SQ;
(2) a double bond between one atom of L₂ and one atom of L₁, wherein
L₁ is C;
L₂ is —(CR₅R₆)ₓ—CR₅═, —C(O)—(CR₅R₆)ₓ—CR₅═, —N(Q)═, —N—, —O—N═, —N(Q)-N═, or —C(O)N(Q)-N═;
(3) a single bond between a first atom of L₂ and a first atom of L₁, and a single bond between a second atom of L₂ and the first atom of L₁, wherein
L₁ is C or C(Rₐ)—(CR₅R₆)ₓ—C(Rₐ);
L₂ has the formula 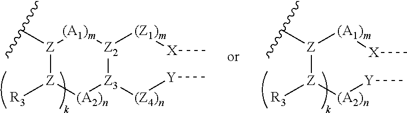
wherein
X is the first atom of L₂, Y is the second atom of L₂, - - - - - represents a single bond to the first atom of L₁, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
Z₁ and Z₄ are each, independently, —O—, —S—, —CH₂—, —CHR⁵—, or
—CR⁵R⁵—;
Z₂ is CH or N;
Z₃ is CH or N;
or Z₂ and Z₃, taken together, are a single C atom;
A₁ and A₂ are each, independently, —O—, —S—, —CH₂—, —CHR⁵—, or —CR⁵R⁵—;
each Z is N, C(R₅), or C(R₃);
k is 0, 1, or 2;
each m, independently, is 0 to 5;
each n, independently, is 0 to 5;
where m and n taken together result in a 3, 4, 5, 6, 7 or 8 member ring;
(4) a single bond between a first atom of L₂ and a first atom of L₁, and a single bond between the first atom of L₂ and a second atom of L₁, wherein
(A) L₁ has the formula: 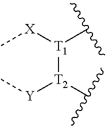
wherein
X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is CH or N;
T₂ is CH or N;
or T₁ and T₂ taken together are C═C;
L₂ is CR₅; or
(B) L₁ has the formula: 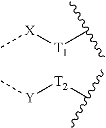
wherein
X is the first atom of L₁, Y is the second atom of L₁, - - - - - represents a single bond to the first atom of L₂, and X and Y are each, independently, selected from the group consisting of —O—, —S—, alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is —CR₅R₆—, —N(Q)-, —O—, or —S—;
T₂ is —CR₅R₆—, —N(Q)-, —O—, or —S—;
L₂ is CR₅ or N;
each of x and y, independently, is 0, 1, 2, 3, 4, or 5;
T₃ is a bond or -L₆-(CR₅R₆)ₘ-L₇-[(CR₅R₆)ₚO]_{q}-L₈-(CR₅R₆)ₙ-L₉- wherein
L₆ is a bond, —CR^{1a}R^{1b}—, —O—, —CO—, —NR^{1d}—, —S—, —P(=Q₁)(Q₂)-, or a combination thereof;
L₇ is a bond, —CR^{1a}R^{1b}—, —O—, —CO—, —NR^{1d}—, —S—, —P(=Q₁)(Q₂)-, or a combination thereof;
L⁸ is a bond, —CR^{1a}R^{1b}—, —O—, —CO—, —NR^{1d}—, —S—, —P(=Q₁)(Q₂)-, or a combination thereof;
L₉ is a bond, —CR^{1a}R^{1b}—, —O—, —CO—, —NR^{1d}—, —S—, —P(=Q₁)(Q₂)-, or a combination thereof;
m is 0 to 10;
n is 0 to 10;
p is 1 to 6;
q is 0 to 2000;
each occurrence of R₅ and R₆ is, independently, H, halo, cyano, hydroxy, amino, alkyl, alkoxy, cycloalkyl, aryl, heteroaryl, or heterocyclyl;
each Q, independently, is H, alkyl, acyl, cycloalkyl, alkenyl, alkynyl, aryl, heteroaryl or heterocyclyl;
each Q₁, independently, is O or S; and
each Q₂, independently, is —OQ, —SQ, —N(Q)Q, alkyl, or alkoxy;
R₃ can have the formula: 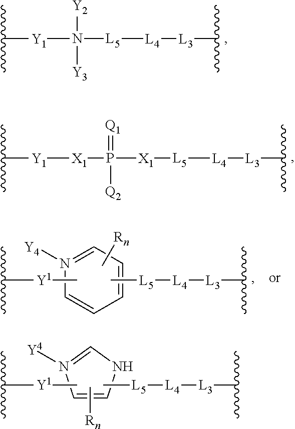
where
Q₁ is O or S;
Y₁ is a bond, alkylene, cycloalkylene, arylene, aralkylene, or alkynylene, wherein
Y₁ is optionally substituted by 0 to 6 Rₙ;
Y₂ is alkyl, cycloalkyl, aryl, aralkyl, or alkynyl, wherein Y₂ is optionally substituted by 0 to 6 Rₙ;
Y₃ is absent, or if present, is alkyl, cycloalkyl, aryl, aralkyl, or alkynyl, wherein
Y₃ is optionally substituted by 0 to 6 Rₙ;
Y₄ is absent, or if present, is alkyl, cycloalkyl, aryl, aralkyl, or alkynyl, wherein
Y₄ is optionally substituted by 0 to 6 Rₙ; or
any two of Y₁, Y₂, and Y₃ are taken together with the N atom to which they are attached to form a 3- to 8-member heterocycle optionally substituted by 0 to 6 Rₙ; or
Y₁, Y₂, and Y₃ are all be taken together with the N atom to which they are attached to form a bicyclic 5- to 12-member heterocycle optionally substituted by 0 to 6 Rₙ;
each Rₙ, independently, is H, halo, cyano, hydroxy, amino, alkyl, alkoxy, cycloalkyl, aryl, heteroaryl, or heterocyclyl;
each X₁, independently, is —O—, —S—, or —(CR⁵R⁶)—;
L₃ is a bond, —N(Q)-, —O—, —S—, —(CR₇R₈)ₐ—, —[O—(CR₅R₆)ₐ]_{c}—, —C(O)—, or a combination of any two of these;
L₄ is a bond, —N(Q)-, —O—, —S—, —(CR₇R₈)ₐ—, —[O—(CR₅R₆)ₐ]_{c}—, —C(O)—, or a combination of any two of these;
L₅ is a bond, —N(Q)-, —O—, —S—, —(CR₇R₈)ₐ—, —[O—(CR₅R₆)ₐ]_{c}—, —C(O)—, or a combination of any two of these;
each occurrence of R₇ and R₈ is, independently, H, halo, cyano, hydroxy, amino, alkyl, alkoxy, cycloalkyl, aryl, heteroaryl, or heterocyclyl;
or two R₇ groups on adjacent carbon atoms are taken together to form a double bond between their respective carbon atoms;
or two R₇ groups on adjacent carbon atoms and two R₈ groups on the same adjacent carbon atoms are taken together to form a triple bond between their respective carbon atoms;
each a, independently, is 0, 1, 2, or 3; wherein
an R₇ or R₈ substituent from any of L₃, L₄, or L₅ is optionally taken with an R₇ or R₈ substituent from any of L₃, L₄, or L₅ to form a 3- to 8-member cycloalkyl, heterocyclyl, aryl, or heteroaryl group;
any one of Y₁, Y₂, or Y₃, is optionally taken together with an R₇ or R₈ group from any of L₃, L₄, and L₅, and atoms to which they are attached, to form a 3- to 8-member heterocyclyl group;
each c, independently, is 0 to 2000;
or a pharmaceutically acceptable salt thereof.

2. The lipid of claim 1, wherein the quinoline moiety includes a 4-aminoquinoline, an 8-aminoquinoline, or a 4-methanolquinoline.

3. The lipid of claim 1, wherein the quinoline moiety includes a chloroquine moiety, an amodiaquine moiety, a primaquine moiety, a pamaquine moiety, a mefloquine moiety, a quinine moiety, or a quinidine moiety.

4. The lipid of claim 1, wherein the moiety that binds to Na+/K+-ATPase includes a cardiac glycoside moiety.

5. The lipid of claim 4, wherein the cardiac glycoside moiety includes a helveticoside moiety, a dihydroouabain moiety, a digitoxigenin moiety, a strophanthidin moiety, a lanatoside C moiety, a ditoxigenin moiety, a digoxin moiety, a ouabain moiety, a proscillaridin A moiety, an arenobufagin moeity, a bufotalin moiety, a cinobufagin moiety, a marinobufagin moiety, a scilliroside moiety, an acetyldigitoxin moiety, an acetyldigoxin moiety, a lanatoside C moiety, a deslanoside moiety, a medigoxin moiety, a gitoformate moiety, a daigremontianin moiety, a cymarin moiety, or a peruvoside moiety.

6. The lipid of claim 1, wherein S₁ has the formula: -G-S₃-Lc, wherein G is a bond, —O— or a glycosidic linkage, S₃ is a steroid structure, and Lc is a lactone.

7. The lipid of claim 6, wherein S₁ has the structure: 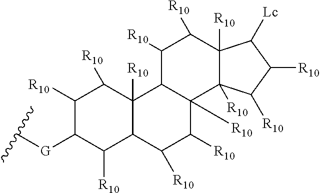
wherein each R₁₀, independently, is H, OH, CH₃, CHO, C(O)CH₃, oxo, or two adjacent R₁₀, taken together, are a double bond or an epoxide.

8. The lipid of claim 7, wherein G is a bond, —O—, or has the formula 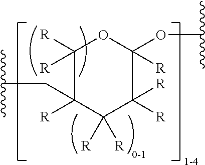
wherein each R, independently, is H, OH, alkyl, alkoxy, acyl, NH₂, or NH-acyl.

9. The lipid of claim 8, wherein Lc has the formula:

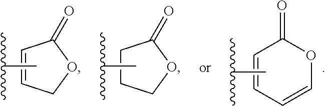

10. The lipid of claim 6, wherein S₁ has the formula:

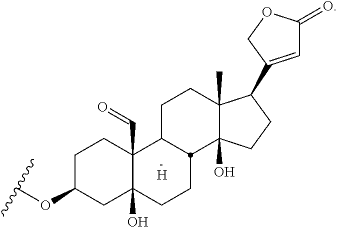

11. The lipid of claim 6, wherein the lipid has the formula:

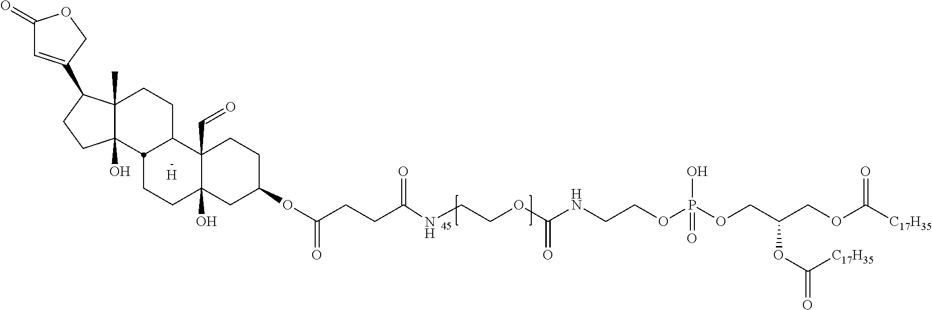

12. The lipid of claim 6, wherein S₁ has the formula:

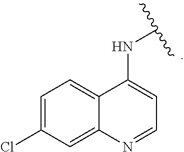

13. The lipid of claim 12, wherein the lipid has the formula:

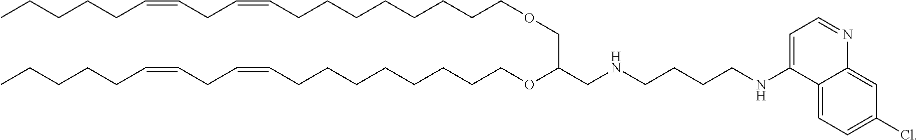

14. A lipid particle comprising a lipid of claim 1.

15. The lipid particle of claim 14, further comprising:
a cationic lipid,
a neutral lipid, and
a lipid capable of reducing aggregation.

16. The lipid particle of claim 15, wherein the neutral lipid is selected from DSPC, DPPC, POPC, DOPE, or SM; the lipid capable of reducing aggregation is a PEG lipid; and the lipid particle further comprises a sterol.

17. The lipid particle of claim 16, further comprising an active agent.

18. The lipid particle of claim 17, wherein the active agent is a nucleic acid selected from the group consisting of a plasmid, an immunostimulatory oligonucleotide, an siRNA, an antisense oligonucleotide, a microRNA, an antagomir, an aptamer, and a ribozyme.

19. A pharmaceutical composition comprising the lipid particle of claim 18 and a pharmaceutically acceptable carrier.

20. A method for enhancing cellular uptake of a nucleic acid, comprising contacting a cell with:
a compound selected from the group consisting of: levodopa, naphazoline hydrochloride, acetohexamide, niclosamide, diprophylline, and isoxicam; and
a lipid particle comprising a nucleic acid.

21. A method for enhancing cytosolic distribution of a nucleic acid, comprising contacting a cell with:
a compound selected from the group consisting of: azaguanine-8, isoflupredone acetate, chloroquine, trimehobenzamide, hydrochloride, isoxsuprine hydrochloride, and diphemanil methylsulfate; and
a lipid particle comprising a nucleic acid.

22. A method of enhancing cellular uptake of a lipid particle, comprising contacting a cell with a lipid particle and a compound that binds a Na+/K+-ATPase.

23. A method of enhancing cellular uptake of a lipid particle, comprising contacting a cell with a lipid particle and a compound that binds a Na+/K+-ATPase, wherein the compound that binds a Na+/K+-ATPase is a lipid of claim 1.
